(19) United States
(12) Patent Application Publication
(10) Pub. No.: US 2021/0386373 A1
(43) Pub. Date: Dec. 16, 2021

(54) ANALYTE DETECTION SYSTEM

(71) Applicant: WearOptimo Pty Ltd, Woolloongabba, Queensland (AU)

(72) Inventors: Mark Anthony Fernance KENDALL, Woolloongabba (AU); Stephen James WILSON, Woolloongabba (AU); Anthony Mark BREWER, Woolloongabba (AU)

(21) Appl. No.: 17/282,334

(22) PCT Filed: Oct. 1, 2019
(86) PCT No.: PCT/AU2019/051065

§ 371 Date: Apr. 1, 2021

(30) Foreign Application Priority Data
Oct. 2, 2018 (AU) 2018903716

(51) Int. Cl. A61B 5/00; A61B 5/145; A61B 5/1473; A61B 90/94

(52) U.S. Cl. CPC A61B 5/685; A61B 5/1451; A61B 5/14735; A61B 5/442; A61B 5/444; A61B 5/6833; A61B 2562/0285; A61B 5/4848; A61B 5/4839; A61B 90/94; A61B 5/0002; A61B 2562/028; A61B 5/6839

(57) ABSTRACT

A system for detecting analytes in a biological subject, the system including at least one substrate including a plurality of microstructures configured to breach a stratum corneum of the subject, and wherein the one or more microstructures are responsive to a presence, absence, level or concentration of analytes to cause a change in appearance thereby indicating that a presence, absence, level or concentration of analytes has been detected.

### BACKGROUND OF THE INVENTION

The present invention relates to a system and method for detecting analytes in a biological subject, and in one particular example, to detecting a presence, absence, level or concentration of analytes in a biological subject by breaching a functional barrier of the subject using microstructures.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Biological markers, such as proteins, antibodies, cells, small chemicals, hormones and nucleic acids, whose presence in excess or deficiency may indicate a diseased state, have been found in blood serum and their levels are routinely measured for research and for clinical diagnosis. Standard tests include antibody analysis for detecting infections, allergic responses, and blood-borne cancer markers (e.g. prostate specific antigen analysis for detecting prostate cancer). The biological markers may originate from many organ systems in the body but are extracted from a single compartment, the venous blood.

However, this is not suitable for all conditions as often blood does not contain key biological markers for diseases originating in solid tissues, and whilst this problem has been partially overcome by taking tissue biopsies, this is time-consuming, painful, risky, costly and can require highly-skilled personnel such as surgeons.

Another serum-rich fluid is the interstitial fluid (ISF) which fills the intercellular spaces in solid tissues and facilitates the passage of nutrients, biomarkers, and excretory products via the blood stream.

WO2005/072630 describes devices for delivering bioactive materials and other stimuli to living cells, methods of manufacture of the device and various uses of the device, including a number of medical applications. The device comprises a plurality of structures which can penetrate a body surface so as to deliver the bioactive material or stimulus to the required site. The structures are typically solid and the delivery end section of the structure is so dimensioned as to be capable of insertion into targeted cells to deliver the bioactive material or stimulus without appreciable damage to the targeted cells or specific sites therein.

The use of microneedle versions of such arrays in sampling fluids is also known. However, the techniques focus on the use of micro-fluidic techniques such as capillary or pumping actions to extract fluid, as described for example in U.S. Pat. Nos. 6,923,764, 6,052,652, 6,591,124, 6,558,361, 6,908,453, and US2005/0261632, US2006/0264782, US2005/0261632, US2005/0261632, U.S. Pat. No. 6,589,202.

However, these systems suffer from a number of drawbacks. Firstly, use of capillary or pumping actions can only be achieved using relatively largely structures, which typically pass through the dermis and consequently can end up sampling blood as opposed to interstitial fluid. This can also cause discomfort and irritation to the subject being sampled. Secondly, the requirement for capillary or pumping actions renders the arrays complex, in structure and requiring power sources resulting in arrays that are difficult and expensive to manufacture, liable to infection, making them unsuitable for general use.

Other in vitro diagnostic devices are known, such as the use of arrays that include silicon nanowires, or other complex detection mechanisms, such as direct radio-frequency detection of nucleotide hybridization to perform the detection. Again, the fabrication of such systems is complex and expensive, again making these unsuitable for practical applications.

U.S. Pat. No. 9,974,471 describes a device and system for measuring and/or monitoring an analyte present on the skin is provided. The system includes a skin-mountable device that may be attached to an external skin surface and a reader device. The skin-mountable device includes a substrate, a plurality of microneedles, and nanosensors. The microneedles are attached to the substrate such that attachment of the substrate to an external skin surface causes to the microneedles to penetrate into the epidermis, intradermis, or dermis. The nanosensors include a detectable label and are configured to interact with a target analyte present in the interstitial fluid in the epidermis, intradermis, or dermis. The reader device is configured to detect the analyte in interstitial fluid via interaction with the skin-mountable device.

US20070142885 describes a system and method for revitalizing aging skin using electromagnetic energy that is delivered using a plurality of needles that are capable of penetrating the skin to desired depths. A particular aspect of the invention is the capability to spare zones of tissue from thermal exposure. This sparing of tissue allows new tissue to be regenerated while the heat treatment can shrink the collagen and tighten the underlying structures. Additionally, the system is capable of delivering therapeutically beneficial substances either through the penetrating needles or through channels that have been created by the penetration of the needles.

U.S. Pat. No. 6,972,013 describes methods for using an electric field to delivery therapeutic or immunizing treatment to a subject by applying non-invasive, user-friendly electrodes to the surface of the skin. Thus, therapeutic or immunizing agents can be delivered into cells of skin for local and systemic treatments or for immunization with optimal gene expression and minimal tissue damage. In particular, therapeutic agents include naked or formulated nucleic acid, polypeptides and chemotherapeutic agents.

U.S. Pat. No. 7,285,090 describes a monitoring apparatus that includes a sensor device and an I/O device in communication with the sensor device that generates derived data using the data from the sensor device. The derived data cannot be directly detected by the associated sensors. Alternatively, an apparatus that includes a wearable sensor device and an I/O device in communication with the sensor device that includes means for displaying information and a dial for entering information. Alternatively, an apparatus for tracking caloric consumption and caloric expenditure data that includes a sensor device and an I/O device in communication with the sensor device. The sensor device includes a processor programmed to generate data relating to caloric expenditure from sensor data. Alternatively, an apparatus for tracking caloric information for an individual that utilizes a plurality of classification identifiers for classifying meals consumed by the individual, each of the classification identifiers having a corresponding caloric amount.

US20110295100 describes methods, systems and/or devices for enhancing conductivity of an electrical signal through a subject's skin using one or more microneedle electrodes are provided. A microneedle electrode may be applied to the subject's skin by placing the microneedle electrode in direct contact with the subject's skin. The microneedles of the microneedle electrode may be inserted into the skin such that the microneedles pierce stratum corneum of the skin up to or through dermis of the skin. An electrical signal passes or is conducted through or across the microneedle electrode and the subject's skin, where impedance of the microneedle electrode is minimal and greatly reduced compared to existing technologies.

WO2009140735 describes an apparatus for use in detecting analytes in a subject, wherein the apparatus includes a number of structures provided on a patch, such that applying the patch to the subject causes at least some of the structures to be inserted into the subject and target one or more analytes and a reagent for detecting the presence or absence of analytes.

U.S. Pat. No. 10,098,574 describes device and system for measuring and/or monitoring an analyte present on the skin is provided. The system includes a skin-mountable device that may be attached to an external skin surface and a reader device. The skin-mountable device includes a substrate, a plurality of micro-needles, and nanosensors encapsulated in the micro-needles. The micro-needles are attached to the substrate such that attachment of the substrate to an external skin surface causes to the micro-needles to penetrate into the skin to contact interstitial fluid. The micro-needles can include a sacrificial agent and are configured to become porous on contact with a solvent, e.g., interstitial fluid, which dissolves at least a portion of the sacrificial agent. The nanosensors encapsulated in the micro-needles include a detectable label and are configured to interact with a target analyte present in the interstitial fluid. The reader device is configured to detect the analyte in interstitial fluid via interaction with the skin-mountable device.

US 2016/0256091 describes a bio information measuring device is provided. The bio information measuring device includes a sensor portion and a needle portion including a plurality of needles projecting from a plurality of openings formed in a surface of the sensor portion. The plurality of needles are configured to pierce tissue, wherein the plurality of needles include a biocompatible organic material which includes an enzyme member that reacts with an analysis material and a conductive polymer for transferring an electrical signal generated as a result of a reaction of the enzyme member with the analysis material.

US 2018/0177439 describes at least one microneedle comprises a hydrogel material that includes a substance that fluoresces when the substance interacts with an analyte. A magnitude of the fluorescence varies as a function of the concentration of the analyte. During use, the hydrogel material is illuminated with illumination light in a first wavelength range while the hydrogel material interfaces with the dermal interstitial fluid layer of a subject, and a photosensor generates an output that corresponds to an amount of light received in a second wavelength range.

US 2007/0276211 describes a biomedical monitor is disclosed. The biomedical monitor has an array of moveable microneedles coated with a first chemical sensing media. The biomedical monitor also has an actuator configured to move at least one microneedle in the array of microneedles from a retracted position to an engaged position whereby the at least one microneedle enters a subject's skin. The biomedical monitor further has an optical system configured to illuminate the at least one microneedle during or after entering the subject's skin and monitor the first chemical sensing media from the at least one microneedle, whereby at least one biomedical characteristic is determined based on at least one spectral property of the monitored first chemical sensing media. A method of monitoring at least one biomedical characteristic is also disclosed.

WO2013058879A2 describes methods, structures, and systems are disclosed for biosensing and drug delivery techniques. In one aspect, a device for detecting an analyte and/or releasing a biochemical into a biological fluid can include an array of hollowed needles, in which each needle includes a protruded needle structure including an exterior wall forming a hollow interior and an opening at a terminal end of the protruded needle structure that exposes the hollow interior, and a probe inside the exterior wall to interact with one or more chemical or biological substances that come in contact with the probe via the opening to produce a probe sensing signal, and an array of wires that are coupled to probes of the array of hollowed needles, respectively, each wire being electrically conductive to transmit the probe sensing signal produced by a respective probe.

US20150208984 describes a transdermal microneedle continuous monitoring system. The continuous system monitoring includes a substrate, a microneedle unit, a signal processing unit and a power supply unit. The microneedle unit at least comprises a first microneedle set used as a working electrode and a second microneedle set used as a reference electrode, the first and second microneedle sets arranging on the substrate. Each microneedle set comprises at least a microneedle. The first microneedle set comprises at least a sheet having a through hole on which a barbule forms at the edge. One of the sheets provides the through hole from which the barbules at the edge of the other sheets go through, and the barbules are disposed separately.

US 2016/0302687 describes a biometric information measuring sensor is provided that includes a base comprising a plurality of bio-marker measuring areas and a plurality of electrodes. Each of the plurality of electrodes is disposed on a respective one of the plurality of bio-marker measuring areas, and each of the plurality of electrodes includes a working electrode and a counter electrode spaced apart from the working electrode. The biometric information measuring sensor also includes a plurality of needles. Each of the needles is disposed on a respective one of the plurality of electrodes. Two or more of the plurality of needles have different lengths.

US 2016/0166184 describes a microneedle device (200) including at least one microneedle (1) having one or more nanowires (203) on a surface of said at least one microneedle. The microneedle device is typically used in a sensor such as a sensor for monitoring glucose levels in the body and the nanowires may have a membrane (207) covering at least part of the nanowires.

KR 20170041375 describes a micro-needle skin patch functionalized with early diagnosis aptamer coated carbon nanotubes of various diseases.

U.S. Pat. No. 8,543,179 describes a biomedical sensor device includes a light source, a probe array, and a photo detector. The light source is configured for emitting infrared radiation. The probe array is contacted to a user's skin to detect an electric wave signal transmitted through the probe array from the skin. The probe array includes a substrate and a plurality of probes mounted on the substrate, wherein the substrate and the probes are non-opaque so that the infrared radiation may be transmitted through the probe array into the skin. The photo detector is configured to detect an infrared signal by measuring the infrared radiation absorption by the skin.

U.S. Pat. No. 8,588,884 describes devices for enhancing conductivity of an electrical signal through a subject's skin using one or more microneedle electrodes are provided. A microneedle electrode may be applied to the subject's skin by placing the microneedle electrode in direct contact with the subject's skin. The microneedles of the microneedle electrode may be inserted into the skin such that the microneedles pierce stratum corneum of the skin up to or through dermis of the skin. An electrical signal passes or is conducted through or across the microneedle electrode and the subject's skin, where impedance of the microneedle electrode is minimal and greatly reduced compared to existing technologies.

US 2016/0051195 describes skin-conformal sensor devices and methods of using the same. As consistent with one or more embodiments, a sensor device includes an upper portion and lower portion. The upper portion includes a plurality of layers including at least one sensor. The lower portion includes a layer of microstructures configured and arranged to interface with skin of a subject and to interlock the skin with the at least one sensor.

US 2005/0261606 describes a device for sampling at least one biological fluid constituent and measuring at least one target constituent within the biological fluid. The device has at least one micro-needle having an open distal end used to penetrate the skin to a depth where pain and bleeding are minimized. The device further includes a hydrophilic gel within the micro-needle for sampling the biological fluid constituents and an electrochemical cell for measuring the concentration of targeted constituents within the sampled biological fluid constituents. In certain embodiments, the electrochemical cell is integrated within the micro-needle whereby the steps of sampling and measuring are performed completely in-situ. In other embodiments, the electrochemical cell is located external to the micro-needle at its proximal end. Constituent sampling and measurement systems, methods and kits are also provided.

WO 2018/124327 describes a method for fabricating an aptamer-coated, microneedle-based diagnostic skin patch and a patch fabricated thereby. The patch has the advantage of attaching a great number of aptamers, which are much smaller in size than antibodies, onto a relatively great number of microneedle tip surfaces. Allowing the attachment of aptamers for various kinds of biomarkers all together thereto, the patch can also simultaneously detect various kinds of materials (multiplexing). Therefore, a microneedle tip-based skin patch can also be used as a protein chip using an aptamer.

### SUMMARY OF THE PRESENT INVENTION

In one broad form, an aspect of the present invention seeks to provide a system for detecting analytes in a biological subject, the system including at least one substrate including a plurality of microstructures configured to breach a stratum corneum of the subject, and wherein the one or more microstructures are responsive to a presence, absence, level or concentration of analytes to cause a change in appearance thereby indicating that a presence, absence, level or concentration of analytes has been detected.

In one broad form, an aspect of the present invention seeks to provide a method for detecting analytes in a biological subject, the method including: using at least one substrate including a plurality of microstructures configured to breach a stratum corneum of the subject; and, using a response of the one or more microstructures to a presence, absence, level or concentration of analytes to determine a change in appearance thereby indicating that a presence, absence, level or concentration of analytes has been detected.

In one embodiment the change in appearance includes at least one of: a change in visual appearance; a change in pattern; a change in electromagnetic properties; a colour change; a change in hue; a change in saturation; a change in lightness; a change in chroma; a change in colourfulness; a change in colour constancy; a change in radiance; a change in luminance; a change in brightness; a change in fluorescence; a change in photoluminescence; a change in phosphorescence; a change in reflectivity; a change in refractivity; a reversible change in appearance; and, an irreversible change in appearance.

In one embodiment the change in appearance is at least one of: a change in the tissue; a change in the substrate; a change on the one or more microstructures; a change in the one or more microstructures; and, is viewable through the substrate.

In one embodiment at least some of the microstructures include at least one of: surface features that cause a change in appearance of the microstructures; a material that undergoes a change in appearance; and, a coating that causes a change in appearance.

In one embodiment at least some of the microstructures include a coating and wherein the coating at least one of: undergoes a change in appearance in response to a presence, absence, level or concentration of analytes; causes a change in appearance of surrounding tissue in response to a presence, absence, level or concentration of analytes; causes a change in appearance of analytes; and, dissolves at least one of: after a defined time period; in response to a presence, absence, level or concentration of analytes; and, upon breaching or penetration of the functional barrier.

In one embodiment at least one of: at least some microstructures are uncoated; at least some microstructures are porous with an internal coating; at least some microstructures are partially coated; different microstructures have different coatings; different parts of microstructures include different coatings; and, at least some microstructures include multiple coatings.

In one embodiment the coating at least one of: interacts with analytes; undergoes a shape change to selectively anchor microstructures; modifies surface properties to at least one of: increase hydrophilicity; increase hydrophobicity; and, minimize biofouling; attracts at least one substance to the microstructures; repels at least one substance from the microstructures; provides a physical structure to at least one of: facilitate penetration of the barrier; strengthen the microstructures; and, anchor the microstructures in the subject; dissolves to at least one of: expose a microstructure; expose a further coating; and, expose a material; provides stimulation to the subject; contains a material; selectively releases a material; acts as a barrier to preclude at least one substance from the microstructures; and, includes at least one of: polyethylene; polyethylene glycol; polyethylene oxide; zwitterions; peptides; hydrogels; and, self-assembled monolayer.

In one embodiment at least some of the microstructures include a material including at least one of: a bioactive material; a reagent for reacting with analytes in the subject; a binding agent for binding with analytes of interest; a material for binding one or more analytes of interest; a probe for selectively targeting analytes of interest; an insulator; a material to reduce biofouling; a material to attract at least one substance to the microstructures; a material to repel at least one substance from the microstructures; a material to attract at least some analytes to the microstructures; and, a material to repel at least some analytes from the microstructures.

In one embodiment the substrate includes a plurality of microstructures and wherein different microstructures are at least one of: differentially responsive to analytes; responsive to different analytes; responsive to different combination of analytes; and, responsive to different concentrations of analytes.

In one embodiment at least some of the microstructures at least one of: attracts at least one substance to the microstructures; repels at least one substance from the microstructures; attracts at least one analyte to the microstructures; and, repels at least one analyte from the microstructures.

In one embodiment an appearance of different parts of the substrate or different microstructures are indicative of at least one of: successful breaching or penetration of the stratum corneum; successful breaching or penetration of a dermal barrier; a presence or absence of different analytes; and, different concentrations of analytes.

In one embodiment the appearance of the substrate includes a pattern used to indicate a presence, absence, level or concentration of analytes, and wherein the pattern includes at least one of: an alphanumeric pattern; or, an iconic pattern.

In one embodiment the substrate and microstructures are at least partially transparent to electromagnetic radiation.

In one embodiment at least one of the substrate and the microstructures include a polymer.

In one embodiment the substrate is at least one of: at least partially flexible; configured to conform to an outer surface of the functional barrier; and, configured to conform to a shape of at least part of a subject.

In one embodiment at least some of the microstructures are plate microstructures that are at least partially tapered and have a substantially rounded rectangular cross sectional shape.

In one embodiment the microstructures include anchor microstructures used to anchor the substrate to the subject and wherein the anchor microstructures at least one of: undergo a shape change; undergo a shape change in response to at least one of substances in the subject and applied stimulation; swell; swell in response to at least one of substances in the subject and applied stimulation; include anchoring structures; have a length greater than that of other microstructures; are rougher than other microstructures; have a higher surface friction than other microstructures; are blunter than other microstructures; are fatter than other microstructures; and, enter the dermis.

In one embodiment at least some of the microstructures have at least one of: a length that is at least one of: less than 2500 μm; less than 1000 μm; less than 750 μm; less than 450 μm; less than 300 μm; less than 250 μm; about 250 μm; about 150 μm; greater than 100 μm; greater than 50 μm; and, greater than 10 μm; a maximum width that is at least one of: less than 2500 μm; less than 1000 μm; less than 750 μm; less than 450 μm; less than 300 μm; less than 250 μm; of a similar order of magnitude to the length; greater than the length; greater than the length; about the same as the length; about 250 μm; about 150 μm; and, greater than 50 μm; and, a maximum thickness that is at least one of: less that the width; significantly less that the width; of a smaller order of magnitude to the length; less than 300 μm; less than 200 μm; less than 50 μm; about 25 μm; and, greater than 10 μm.

In one embodiment at least some of the microstructures include at least one of: a shoulder that is configured to abut against the stratum corneum to control a depth of penetration; and, a shaft extending from a shoulder to the tip, the shaft being configured to control a position of the tip in the subject.

In one embodiment the microstructures have at least one of: a density that is at least one of: less than 5000 per cm²; greater than 100 per cm²; and, about 600 per cm²; and, a spacing that is at least one of: less than 1 mm; about 0.5 mm; about 0.2 mm; about 0.1 mm; and, more than 10 μm.

In one embodiment microstructures include an at least partially electromagnetically transparent material.

In one embodiment microstructures include an electromagnetically opaque layer including ports to allow electromagnetic radiation to be emitted from or received by the ports.

In one embodiment microstructures include electromagnetically reflective layers to allow electromagnetic radiation to be conducted to and from designated ports.

In one embodiment the microstructures are configured to deliver stimulation, and wherein the stimulation is at least one of: biochemical; chemical; mechanical; magnetic; thermal; electrical; electromagnetic; and, optical.

In one embodiment the system includes an actuator configured to apply a force to the substrate to at least one of pierce and penetrate the stratum corneum.

In one embodiment the actuator is at least one of: an electromagnetic actuator; a vibratory motor; a piezoelectric actuator; and, a mechanical actuator.

In one embodiment the actuator is configured to apply at least one of: a biasing force; a vibratory force; and, a single continuous force.

In one embodiment the force at least one of: includes a continuous force that is at least one of: greater than 1 N; less than 10 N; and, about 2.5 to 5 N; includes a vibratory force that is at least one of: at least 1 mN; about 200 mN; and, less than 1000 mN; and, is applied at a frequency that is at least one of: at least 10 Hz; about 100 to 200 Hz; and, less than 1 kHz.

In one embodiment the substrate is at least one of: secured to the subject; secured to the subject using anchor microstructures; secured to the subject using an adhesive patch; and, secured to the subject using a strap.

In one embodiment the system is configured to perform measurements over a time period and wherein the microstructures are configured to remain in the subject during the time period.

In one embodiment the time period is at least one of: at least one minute; at least one hour; at least one day; and, at least one week.

In one embodiment change in appearance is viewable upon exposure to electromagnetic radiation having a defined wavelength range, the defined wavelength range including at least one of: infrared radiation; ultraviolet radiation; and, visible radiation.

In one embodiment the change in appearance is detectable using an imaging device.

In one embodiment the system includes one or more electronic processing devices that: receive an image of the substrate from an imaging device; and, analyse the image to determine an indicator indicative of a presence, absence, level or concentration of analytes.

In one embodiment the one or more electronic processing devices: control a radiation source to selectively expose the substrate to electromagnetic radiation; and, control the imaging device to capture an image in response to exposure of the substrate to electromagnetic radiation.

In one embodiment the image is captured at least one of: concurrently with exposure of the substrate to electromagnetic radiation; and, a defined time after exposure of the substrate to electromagnetic radiation.

In one embodiment the system is at least partially wearable.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which: —

FIG. 1 is a schematic diagram of an example of a system for detecting analytes in a biological subject;

FIG. 2 is a flow chart of an example of a process for detecting analytes in a biological subject;

FIG. 3A is a schematic diagram of a further example of a system for performing measurements on a biological subject;

FIG. 3B is a schematic underside view of an example of a patch for the system of FIG. 3A;

FIG. 3C is a schematic plan view of an example of a hexagonal grid microstructure array;

FIG. 3D is a schematic plan view of an example of an interspersed hexagonal grid microstructure array;

FIG. 3E is a schematic plan view of an example of patterned grid microstructure array;

FIG. 3F is a schematic side view of an example of a microstructure;

FIG. 3G is a schematic side view of a modified version of the microstructure of FIG. 3F;

FIG. 3H is a schematic underside view of an alternative example of a patch;

FIG. 3I is a schematic side view of the patch of FIG. 3H;

FIG. 3J is a schematic side view of an example of a housing arrangement for the system of FIG. 3A;

FIG. 3K is a schematic plan view of the housing arrangement of FIG. 3J;

FIG. 3L is a schematic side view of an example of a flexible segmented substrate arrangement;

FIG. 3M is a schematic side view of a further example of a flexible segmented substrate arrangement;

FIG. 3N is a schematic side view of a further example of a flexible segmented substrate arrangement;

FIG. 3O is a schematic side view of a further example of a flexible segmented substrate arrangement;

FIG. 3P is a schematic side view of an example of an actuator configuration;

FIG. 3Q is a schematic side view of a further example actuator arrangement;

FIG. 3R is a schematic side view of an example of microstructures configured to produce an observable change prior to swelling;

FIG. 3S is a schematic side view of the microstructures of FIG. 3R after swelling;

FIG. 3T is a schematic plan view of microstructures of FIG. 3R prior to swelling;

FIG. 3U is a schematic plan view of the microstructures of FIG. 3R after swelling;

FIG. 3V is a schematic plan view of a further example of microstructures configured to produce an observable change after to swelling;

FIG. 3W is a schematic plan view of the microstructures of FIG. 3V prior to swelling;

FIG. 4A is a schematic side view of a first example of a microstructure configuration;

FIG. 4B is a schematic side view of a second example of a microstructure configuration;

FIGS. 4C to 4H are schematic diagrams illustrating example microstructure cross sectional shapes;

FIG. 5A is a schematic side view of an example of a plate microstructure;

FIG. 5B is a schematic front view of the microstructure of FIG. 5A;

FIG. 5C is a schematic side view of a coated version of the microstructure of FIG. 5A;

FIG. 5D is a schematic front view of an example of a blade microstructure;

FIG. 5E is a schematic perspective topside view of an example of substrate including blade microstructures;

FIG. 5F is a schematic side view of another example of a microstructure;

FIG. 5G is a schematic front view of the microstructure of FIG. 5F;

FIG. 5H is a schematic side view of an example of a first step of a microstructure construction technique;

FIG. 5I is a schematic side view of an example of a second step of a microstructure construction technique;

FIG. 5J is a schematic side view of an example of a third step of a microstructure construction technique;

FIG. 5K is a schematic side view of a first example of a microstructure configuration created using the construction technique of FIGS. 5H to 5J;

FIG. 5L is a schematic side view of a second example of a microstructure configuration created using the construction technique of FIGS. 5H to 5J;

FIG. 5M is a schematic side view of a specific example of a plate microstructure;

FIG. 5N is a schematic perspective view of the plate microstructure of FIG. 5M;

FIG. 5O is a schematic side view of an example of microstructures inserted into a subject for epidermal measurement;

FIG. 5P is a schematic side view of an example of microstructures inserted into a subject for dermal measurement;

FIG. 6 is a flow chart of an example of a process for detecting analytes;

FIG. 7 is a schematic diagram of an example of a monitoring device;

FIG. 8 is a flow chart of an example process of detecting analytes using the monitoring device of FIG. 7;

FIG. 9 is a schematic diagram of an example of a distributed computer architecture;

FIG. 10 is a schematic diagram of an example of a processing system;

FIG. 11 is a schematic diagram of an example of a client device;

FIG. 12 is a flow chart of an example of a process for creating a subject record;

FIGS. 13A and 13B are a flow chart of a specific example of a process for performing measurements in a biological subject;

FIGS. 14A to 14P are schematic diagrams illustrating steps in an example manufacturing process;

FIGS. 15A to 15D are micrograph images of examples of microstructures manufactured using the approach of FIGS. 14A to 14P;

FIGS. 16A to 16L are schematic diagrams illustrating steps in an example manufacturing process;

FIGS. 17A and 17B are micrograph images of examples of microstructures manufactured using the approach of FIGS. 16A to 16L;

FIGS. 17C and 17D are micrograph images of further examples of microstructures manufactured using the approach of FIGS. 16A to 16L;

FIGS. 18A and 18B are micrograph images of examples of partially coated microstructures;

FIG. 19A is an image of a resazurin-coated clear microstructure patch;

FIG. 19B is an image of the patch of FIG. 19A after exposure to a cell broth; and,

FIG. 19C is a graph of UV-vis measurements taken through the microstructures of the patch of FIG. 19A, prior to coating, after coating and following exposure;

FIG. 20A is an image of a microstructure patch application site on a human forearm skin immediately post-removal;

FIG. 20B is a Scanning Electron Micrograph of a microstructure after application to human skin;

FIG. 21A is a graph of example qualitative scores of erythema at microstructure patch application sites on human forearm skin from a first study;

FIG. 21B is a graph of example qualitative scores of erythema at microstructure patch application sites on human forearm skin from a second study;

FIG. 22A is a Scanning Electron Micrographs of microstructure prior to application into human forearm skin;

FIG. 22B is a Scanning Electron Micrographs of the microstructure of FIG. 22A post application into human forearm skin;

FIG. 22C is a Scanning Electron Micrographs of a microstructure patch post application into human forearm skin;

FIG. 22D is a Scanning Electron Micrographs of microstructure prior to application into human forearm skin;

FIG. 22E is a Scanning Electron Micrographs of the microstructure of FIG. 22D post application into human forearm skin;

FIG. 22F is a Scanning Electron Micrographs of a microstructure patch post application into human forearm skin;

### DETAILED DESCRIPTION OF THE PREFERRED EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The terms “about” and “approximately” are used herein to refer to conditions (e.g. amounts, levels, concentrations, time, etc.) that vary by as much as 20% (i.e. ±20%), especially by as much as 10%, 9%, 8%, 7%, 6%, 5%, 4%, 3%, 2% or 1% to a specified condition.

As used herein, the term “analyte” refers to a naturally occurring and/or synthetic compound, which is a marker of a condition (e.g., drug abuse), disease state (e.g., infectious diseases), disorder (e.g., neurological disorders), or a normal or pathologic process that occurs in a subject (e.g., drug metabolism), or a compound which can be used to monitor levels of an administered or ingested substance in the subject, such as a medicament (substance that treats, prevents and/or alleviates the symptoms of a disease, disorder or condition, e.g., drug, vaccine etc.), an illicit substance (e.g. illicit drug), a non-illicit substance of abuse (e.g. alcohol or prescription drug taken for non-medical reasons), a poison or toxin (including an environmental contaminant), a chemical warfare agent (e.g. nerve agent, and the like) or a metabolite thereof. The term “analyte” can refer to any substance, including chemical and/or biological agents that can be measured in an analytical procedure, including nucleic acids, proteins, illicit drugs, explosives, toxins, pharmaceuticals, carcinogens, poisons, allergens, and infectious agents, which can be measured in an analytical procedure. The analyte may be a compound found directly in a sample such as biological tissue, including body fluids (e.g. interstitial fluid), from a subject, especially in the dermis and/or epidermis. In particular embodiments, the analyte is a compound found in the interstitial fluid. In some embodiments, the analyte is a compound with a molecular weight in the range of from about 30 Da to about 100 kDa, especially about 50 Da to about 40 kDa. Other suitable analytes are as described herein.

As used herein, the term “and/or” refers to and encompasses any and all possible combinations of one or more of the associated listed items, as well as the lack of combinations when interpreted in the alternative (or).

As used herein, the term “aptamer” refers to a single-stranded oligonucleotide (e.g. DNA or RNA) that binds to a specific target molecule, such as an analyte. An aptamer may be of any size suitable for binding such target molecule, such as from about 10 to about 200 nucleotides in length, especially from about 30 to about 100 nucleotides in length.

The term “bind” and variations such as “binding” are used herein to refer to an interaction between two substances, such as an analyte and an aptamer or an analyte and a molecularly imprinted polymer. The interaction may be a covalent or non-covalent interaction, particularly a non-covalent interaction.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “plurality” is used herein to refer to more than one, such as 2 to 1×10¹⁵ (or any integer therebetween) and upwards, including 2, 10, 100, 1000, 10000, 1×10⁶, 1×10⁷, 1×10⁸, 1×10⁹, 1×10¹⁰, 1×10¹¹, 1×10¹², 1×10¹³, 1×10¹⁴, 1×10¹⁵, etc. (and all integers therebetween).

As used herein, the term “predetermined threshold” refers to a value, above or below which indicates the presence, absence or progression of a disease, disorder or condition; the presence or absence of an illicit substance or non-illicit substance of abuse; or the presence or absence of a chemical warfare agent, poison and/or toxin. For example, for the purposes of the present invention, a predetermined threshold may represent the level or concentration of a particular analyte in a corresponding sample from an appropriate control subject, such as a healthy subject, or in pooled samples from multiple control subjects or medians or averages of multiple control subjects. Thus, a level or concentration above or below the threshold indicates the presence, absence or progression of a disease, disorder or condition; the presence or absence of an illicit substance or non-illicit substance of abuse; or the presence or absence of a chemical warfare agent, poison and/or toxin, as taught herein. In other examples, a predetermined threshold may represent a value larger or smaller than the level or ratio determined for a control subject so as to incorporate a further degree of confidence that a level or ratio above or below the predetermined threshold is indicative of the presence, absence or progression of a disease, disorder or condition; the presence or absence of an illicit substance or non-illicit substance of abuse; or the presence or absence of a chemical warfare agent, poison and/or toxin. Those skilled in the art can readily determine an appropriate predetermined threshold based on analysis of samples from appropriate control subjects.

The terms “selective” and “selectivity” as used herein refer to molecularly imprinted polymers or aptamers that bind an analyte of interest without displaying substantial binding of one or more other analytes. Accordingly, a molecularly imprinted polymer or aptamers that is selective for an analyte, such as troponin or a subunit thereof, exhibits selectivity of greater than about 2-fold, 5-fold, 10-fold, 20-fold, 50-fold, 100-fold or greater than about 500-fold with respect to binding of one or more other analytes.

The term “subject” as used herein refers to a vertebrate subject, particularly a mammalian subject, for whom monitoring and/or diagnosis of a disease, disorder or condition is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; bats; and captive wild animals such as foxes, deer and dingoes. In particular, the subject is a human.

### System for Performing Measurements

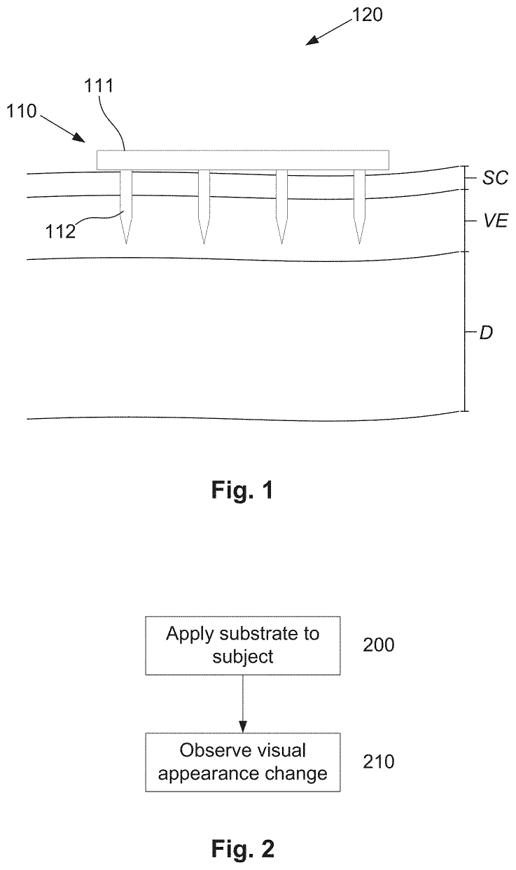

An example of a system for detecting analytes in a biological subject will now be described with reference to FIG. 1.

In this example, the system includes at least one substrate 111 having one or more microstructures 112. In use, the microstructures are configured to breach a functional barrier associated with a subject. In the current example, the functional barrier is the stratum corneum SC, and the microstructures are configured to breach the stratum corneum SC by penetrating the stratum corneum SC and entering at least the viable epidermis VE. In one particular example, the microstructures are configured to not penetrate a boundary between the viable epidermis VE and the dermis D, although this is not essential and structures that penetrate into the dermis could be used as will be described in more detail below.

Whilst this example is described with respect to breaching of the stratum corneum SC, it will be appreciated that this is not essential, and the techniques could equally be applied to other functional barriers. In this regard, a functional barrier will be understood to include any structure, boundary, or feature, whether physical or otherwise, that prevents passage of signals, and/or analytes, such as biomarkers. For example, functional barriers could include one or more layers, a mechanical discontinuity, such as a discrete change in tissue mechanical properties, a tissue discontinuity, a cellular discontinuity, a neural barrier, a sensor barrier, a cellular layer, skin layers, mucosal layers, internal or external barriers, an inner barrier within an organ, an outer barrier of organs other than the skin, epithelial layers or endothelial layers, or the like. Functional barriers could also include other internal layers or boundaries, including optical barriers such as a melanin layer, electrical barriers, molecular weight barriers that prevent passage of a biomarkers with certain molecular weights, a basal layer boundary between the viable epidermis and dermis, or the like.

The nature of the microstructure will vary depending upon the preferred implementation. In one example, the microstructures could include needles, but this is not essential and more typically structures, such as plates, blades, or the like, are used, as will be described in more detail below.

The substrate and microstructures could be manufactured from any suitable material, and the material used may depend on the intended application, for example depending on whether there is a requirement for the structures to be at least partially transparent to electromagnetic radiation, or the like. The substrate can form part of a patch 110, which can be applied to a subject, although other arrangements could be used for example, having the substrate form part of a housing containing other components.

The one or more microstructures are configured so that they are responsive to a presence, absence, level or concentration of analytes to cause a change in appearance thereby indicating that a presence, absence, level or concentration of analytes have been detected.

The change in appearance can be of any appropriate form, and could include a change in the appearance of the substrate itself, a change in the appearance of one or more of the microstructures, or a coating applied to the microstructures. As a further alternative, the change could be a change in the appearance of tissue surrounding the microstructures, for example caused by release of a dye, or a change in the appearance of analytes, for example by binding the analytes to fluorophores, or similar.

In general, the change in appearance is a change in visual appearance, which can be observed using the naked eye. This can include a change in colour or other electromagnetic properties which can be perceived by a user, and may include one or more patterns or icons to indicate specifics of the detection, such as the analytes that have been detected, and whether the detection is a presence, absence or specific concentration. However, the change in appearance could also be a change in appearance that cannot necessarily be viewed with the naked eye, and may require an additional detection mechanism, such as use of an imaging device and/or exposure to set wavelengths of electromagnetic radiation.

It will therefore be appreciated that the term change in appearance should be understood to encompass a change in electromagnetic properties that can be detected by the naked eye, and/or using a suitable detector, either under ambient illumination and/or under exposure to specific wavelength ranges of electromagnetic radiation. For example, it may be necessary to expose the substrate and microstructures to infrared or ultraviolet radiation to induce a visual response, or vice versa.

An example of the manner in which this is performed will now be described with reference to FIG. 2.

In particular, in this example, at step 200, the substrate is applied to the subject so that the one or microstructures breach, and in one example, penetrate the functional barrier. For example, when applied to skin, the microstructures could penetrate the stratum corneum and enter the viable epidermis as shown in FIG. 1. This could be achieved manually and/or through the use of an actuator, to help ensure successful penetration.

At step 210, any change in the appearance is then used to determine if analytes have been detected. In this regard, the detection could be a simple binary process, indicating whether or not analytes are present, although alternatively, the detection process could give a non-binary response, for example to indicate a concentration of analytes.

In any event, it will be appreciated that the above described system operates by providing microstructures that are configured to breach a barrier, such as the stratum corneum, allowing these to be used to detect analytes within the subject.

In contrast to traditional approaches, breaching and/or at least partially penetrating a functional barrier allows measurements to be performed from within or under the barrier and results in a significant improvement in the detection process. In particular, this ensures that the detection accurately reflects conditions within the human body, such as the presence, absence, level or concentration of analytes, or the like, as opposed to traditional external measurements, which are unduly influenced by the environment outside the barrier, such as the physical properties of the skin surface, such as the skin material properties, presence or absence of hair, sweat, or the like.

For example, this allows accurate measurement of high molecular weight biomarkers to be performed, which would otherwise only pass through the skin poorly. A good example of this, is glucose, which whilst present externally, such as in sweat, is typically only present in low concentrations, and often time delayed, meaning the concentration in sweat does not necessarily reflect current glucose levels within the body. In contrast, by breaching the barrier, in this case the stratum corneum, this allows far more accurate measurements to be performed. It will be appreciated that similar considerations apply to a wide range of different analytes, and associated barriers that otherwise prevent accurate measurement of the analytes.

Additionally, in some examples, the microstructures only penetrate the barrier a sufficient distance to allow a measurement to be made. For example, in the case of skin, the microstructures are typically configured to enter the viable epidermis and not enter the dermal layer. This results in a number of improvements over other invasive techniques, including avoiding issues associated with penetration of the dermis, such as pain caused by exposure of nerves, erythema, petechiae, or the like. Avoiding penetrating the dermal boundary also significantly reduces the risk of infection, allowing the microstructures to remain embedded for prolonged periods of time, such as several days, which in turn can be used to perform longitudinal monitoring over a prolonged time periods. However, in some instances, such as when detecting troponin or a subunit thereof, penetration of the dermal barrier may be required.

It will be appreciated that the ability of the microstructures to remain in-situ is particularly beneficial, as this ensures that measurements are made at the same site within the subject, which reduces inherent variability arising from inaccuracies of replacement of measuring equipment which can arise using traditional techniques. Despite this, it will be appreciated that the system can be used in other manners, for example to perform single time point monitoring or the like.

In one example, this allows the arrangement to be provided as part of a wearable device, enabling measurements to be performed that are significantly better than existing surface based measurement techniques, for example by providing access to signals or biomarkers that cannot otherwise pass through the barrier, but whilst allowing measurements to be performed whilst the subject is undergoing normal activities and/or over a prolonged period of time. This in turn enables measurements to be captured that are more accurately reflective of the health or other status of the subject. For example, this allows variations in a subject's condition during a course of the day to be measured, and avoids measurements being made under artificial conditions, such as within a clinic, which are not typically indicative of the actual condition of the subject. This also allows monitoring to be performed substantially continuously, which can allow conditions to be detected as they arise, for example, in the case of myocardial infarction, cardiovascular disease, vomiting, diarrhoea, or similar, which can allow more rapid intervention to be sought.

The above described system can be applied to any part of the body, and hence could be used with a wide range of different functional barriers. For example, the functional barrier could be an internal or external barrier, a skin layer, a mucosal layer, an inner barrier within an organ, an outer barrier of an organ, an epithelial layer, an endothelial layer, a melanin layer, an optical barrier, an electrical barrier, molecular weight barrier, basal layer or the stratum corneum. Thus, the microstructures could be applied to the buccal mucosa, the eye, or another epithelial layer, endothelial layer, or the like. The following examples will focus specifically on application to the skin, with the functional barrier including some or all of the stratum corneum, but it will be appreciated that this is intended to be illustrative and is not intended to be limiting.

Further variations will become apparent from the following description.

In general, the change in appearance includes a change in visual appearance, optionally including a change in pattern. The change in appearance can be achieved as a result of a change in electromagnetic properties, such as a colour change, a change in reflectivity, a change in refractivity, a change in fluorescence, a change in photoluminescence, a change in phosphorescence, a change in hue, a change in saturation, a change in lightness, a change in chroma, a change in colourfulness, a change in colour constancy, a change in radiance, a change in luminance, a change in brightness, or the like. In one example, this allows changes to be viewed by the eye, optionally using particular illumination, such as electromagnetic radiation having a particular frequency range, polarisation, or the like. In another example, the change could be measured using a suitable sensing arrangement, such as an optical sensor, including a photodiode, CCD (Charge Coupled Device) array, or similar.

The change in appearance could be reversible, or could be permanent, depending on the mechanism used to induce the change in appearance. As a result, the change in appearance could be indicative of current analyte concentrations, or could be indicative of a prior concentration, for example when detection was initially performed.

The change in appearance could be achieved using a variety of mechanisms. For example, the change in appearance could be a change in the substrate but more typically is a change in or on the one or more microstructures, which is viewable through the substrate. This can be achieved in a wide variety of manners. For example, the microstructures could incorporate a material that undergoes an appearance change when exposed to analytes.

The nature of such an additive will vary depending on the preferred implementation and could include a bioactive material, a reagent for reacting with analytes in the subject, a binding agent for binding with analytes of interest, a material for binding one or more analytes of interest, a probe for selectively targeting analytes of interest, a material to reduce biofouling, a material to attract at least one substance to the microstructures, a material to repel at least one substance from the microstructures, a material to attract at least some analytes to the microstructure, or a material to repel analytes. In this regard, substances could include any one or more of cells, fluids, analytes, or the like.

The material could be incorporated into the microstructure itself, for example by impregnating the microstructures during manufacture, or alternatively, the microstructures could include a coating that undergoes a change in appearance, and/or dissolves. Accordingly, it will be appreciated that at least some of the microstructures can be coated with a coating such as a bioactive material, which can be used in order to target specific analytes of interest, allowing these to bind or otherwise attach to the microstructure, so that these can then be detected in situ using the change in visual appearance.

As a further alternative, the microstructures could include surface features, such as photonic crystals, or other structures which have optical properties depending on a surface geometry, such as diffraction gratings or similar. In this case, the microstructure could be configured to undergo a shape change, such as swelling or dissolving, which in turn alters the configuration of the surface structure, and hence the appearance.

A further variation could include altering the appearance of the subject, for example to change the appearance of tissue surrounding the patch, allowing this to be viewed via the substrate. This could be achieved by releasing a dye in the presence of analytes, or could be achieved by binding markers, such as fluorophores to analytes, in turn allowing these to be viewed.

The analyte may be any compound able to be detected in the epidermis and/or dermis. In particular embodiments, the analyte is a marker of a condition, disease, disorder or a normal or pathologic process that occurs in a subject, or a compound which can be used to monitor levels of an administered substance in the subject, such as a medicament (e.g., drug, vaccine), an illicit substance (e.g. illicit drug), a non-illicit substance of abuse (e.g. alcohol or prescription drug taken for non-medical reasons), a poison or toxin, a chemical warfare agent (e.g. nerve agent, and the like) or a metabolite thereof. Suitable analytes include, but are not limited to a:
nucleic acid, including DNA and RNA, including short RNA species including microRNA, siRNA, snRNA, shRNA and the like;
antibody, or antigen-binding fragment thereof, allergen, antigen or adjuvant;
chemokine or cytokine;
hormone;
parasite, bacteria, virus, or virus-like particle, or a compound therefrom, such as a surface protein, an endotoxin, and the like;
epigenetic marker, such as the methylation state of DNA, or a chromatin modification of a specific gene/region;
peptide;
polysaccharide (glycan);
polypeptide;
protein; and
small molecule.

In particular embodiments, the analyte of interest is selected from the group consisting of a nucleic acid, antibody, peptide, polypeptide, protein and small molecule; especially a polypeptide and protein; most especially a protein.

The analyte may be a biomarker, which is a biochemical feature or facet that can be used to measure the progress of a disease, disorder or condition or the effects of treatment of a disease, disorder or condition. The biomarker may be, for example, a virus or a compound therefrom, a bacterium or a compound therefrom, a parasite or a compound therefrom, a cancer antigen, a cardiac disease indicator, a stroke indicator, an Alzheimer's disease indicator, an antibody, a mental health indicator, and the like.

Alternatively, the analyte may be a compound which can be used to monitor levels of an administered or ingested substance in the subject, such as a medicament (e.g., drug, vaccine), an illicit substance (e.g. illicit drug), a non-illicit substance of abuse (e.g. alcohol or prescription drug taken for non-medical reasons), a poison or toxin, a chemical warfare agent (e.g. nerve agent, and the like) or a metabolite thereof.

In some embodiments, the analyte is a protein selected from the group consisting of troponin or a subunit thereof, an enzyme (e.g. amylase, creatinine kinase, lactate dehydrogenase, angiotensin II converting enzyme), a hormone (e.g. follicle-stimulating hormone or luteinising hormone), cystatin C, C-reactive protein, TNFα, IL-6, ICAM1, TLR2, TLR4, presepsin, D-dimer, a viral protein (e.g. non-structural protein 1 (NS1)), a bacterial protein, a parasitic protein (e.g. histone rich protein 2 (HRP2)), an antibody (e.g. an antibody produced in response to an infection, such as a bacterial or viral infection including an influenza infection) and botulinum toxin or a metabolite or subunit thereof; especially troponin or a subunit thereof, amylase, creatinine kinase, lactate dehydrogenase, angiotensin II converting enzyme, follicle-stimulating hormone, luteinising hormone, cystatin C, C-reactive protein, TNFα, IL-6, ICAM1, TLR2, TLR4, presepsin, D-dimer, botulinum toxin or a metabolite or subunit thereof. In particular embodiments, the analyte is troponin or a subunit thereof; especially troponin I, troponin C or troponin T; most especially troponin I.

The analyte may be a small molecule, non-limiting examples of which include a hormone (e.g. cortisol or testosterone), neurotransmitter (e.g. dopamine), amino acid, creatinine, an aminoglycoside (e.g. kanamycin, gentamicin and streptomycin), an anticonvulsant (e.g. carbamazepine and clonazepam), an illicit substance (e.g. methamphetamine, amphetamine, 3,4-methylenedioxymethamphetamine (MDMA), N-ethyl-3,4-methylenedioxyamphetamine (MDEA), 3,4-methyl enedioxy-amphetamine (MDA), cannabinoids (e.g. delta-9-tetrahydrocannabinol, 11-hydroxy-delta-9-tetrahydrocannabinol, 11-nor-9-carboxydelta-9-tetrahydrocannabinol), cocaine, benzoylecgonine, ecgonine methyl ester, cocaethylene, ketamine, and the opiates (e.g. heroin, 6-monoacetylmorphine, morphine, codeine, methadone and dihydrocodeine), an anticoagulant (e.g. warfarin), a chemical warfare agent, poison or toxin such as blister agents (e.g. cantharidin, furanocoumarin, sulfur mustards (e.g. 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl)sulfide, bis(2-chloroethylthio)methane, bis(2-chloroethylthiomethyl)ether, bis(2-chloroethylthioethyl)ether), nitrogen mustards (e.g. bis(2-chloroethyl)ethylamine, bis(2-chloroethyl)methylamine and tris(2-chloroethyl)amine) and phosgene oxime), arsenicals (e.g. ethyldichloroarsine, methyldichloroarsine, phenyldichloroarsine and 2-chlorovinyldichloroarsine) and urticants e.g. phosgene oxime), blood agents (e.g. cyanogen chloride, hydrogen cyanide and arsine), choking agents (e.g. chlorine, chloropicrin, diphosgene and phosgene), nerve agents (e.g. tabun, sarin, soman, cyclosarin, novichok agents, 2-(dimethylamino)ethyl-N,N-dimethylphosphoramidofluoridate (GV), (S)-(ethyl{[2-(diethylamino)ethyl]sulfanyl}(ethyl)phosphinate) (VE), O,O-diethyl-S-[2-(diethylamino)ethyl]phosphorothioate (VG), S-[2-(diethylamino)ethyl]-O-ethyl methylphosphonothioate (VM), ethyl({2-[bis(propan-2-yl)amino]ethyl}sulfanyl)(methyl)phosphinate (VX), tetrodotoxin and saxitoxin), animal venom component (e.g. tetrodotoxin and saxitoxin), cyanide, arsenic, a tropane alkaloid (e.g. atropine, scopolamine and hyoscyamine), a piperidine alkaloid (e.g. coniine, N-methylconiine, conhydrine, pseudoconhydrine and gamma-coniceine), a curare alkaloid (e.g. tubocurarine), nicotine, caffeine, quinine, strychnine, brucine, aflatoxin), and the like or a metabolite thereof. In some embodiments the small molecule is selected from the group consisting of cortisol, testosterone, creatinine, dopamine, kanamycin, gentamicin, streptomycin, carbamazepine, clonazepam, methamphetamine, amphetamine, MDMA, MDEA, MDA, delta-9-tetrahydrocannabinol, 11-hydroxy-delta-9-tetrahydrocannabinol, 11-nor-9-carboxydelta-9-tetrahydrocannabinol, cocaine, benzoylecgonine, ecgonine methyl ester, cocaethylene, ketamine, heroin, 6-monoacetylmorphine, morphine, codeine, methadone, dihydrocodeine, warfarin, cantharidin, furanocoumarin, 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl)sulfide, bis(2-chloroethylthio)methane, bis(2-chloroethylthiomethyl)ether, bis(2-chloroethylthioethyl)ether), bis(2-chloroethyl)ethylamine, bis(2-chloroethyl)methylamine and tris(2-chloroethyl)amine), phosgene oxime, ethyldichloroarsine, methyldichloroarsine, phenyldichloroarsine, 2-chlorovinyldichloroarsine, phosgene oxime, cyanogen chloride, hydrogen cyanide, arsine, chlorine, chloropicrin, diphosgene, phosgene, tabun, sarin, soman, cyclosarin, novichok agents, 2-(dimethylamino)ethyl-N,N-dimethylphosphoramidofluoridate (GV), (S)-(ethyl {[2-(diethylamino)ethyl]sulfanyl}(ethyl)phosphinate) (VE), O,O-diethyl-S-[2-(diethylamino)ethyl]phosphorothioate (VG), S-[2-(diethylamino)ethyl]-O-ethyl methylphosphonothioate (VM), ethyl({2-[bis(propan-2-yl)amino]ethyl}sulfanyl)(methyl)phosphinate (VX), tetrodotoxin, saxitoxin, cyanide, arsenic, atropine, scopolamine, hyoscyamine, coniine, N-methylconiine, conhydrine, pseudoconhydrine, gamma-coniceine, tubocurarine, nicotine, caffeine, quinine, strychnine, brucine, aflatoxin and metabolites thereof.

In some embodiments, the analyte is a peptide, non-limiting examples of which include a hormone (e.g. oxytocin, gonadotropin-releasing hormone and adrenocorticotropic hormone), B-type natriuretic peptide, N-terminal pro B-type natriuretic peptide (NT-proBNP) and an animal venom component (e.g. a peptidic component of spider, snake, scorpion, bee, wasp, ant, tick, conesnail, octopus, fish (e.g stonefish) and jellyfish venom) or a metabolite thereof. In particular embodiments, the peptide is oxytocin, gonadotropin-releasing hormone, adrenocorticotropic hormone, B-type natriuretic peptide or NT-proBNP.

In some embodiments, the analyte is a polysaccharide (glycan), suitable non-limiting examples of which include inulin, endotoxins (lipopolysaccharides), anticoagulants (e.g. heparin) and metabolites thereof.

In some embodiments, the analyte is an illicit substance or a non-illicit substance of abuse or a metabolite thereof. Suitable illicit substances include, but are not limited to, methamphetamine, amphetamine, 3,4-methylenedioxymethamphetamine (MDMA), N-ethyl-3,4-methylenedioxyamphetamine (MDEA), 3,4-methylenedioxy-amphetamine (MDA), cannabinoids (e.g. delta-9-tetrahydrocannabinol, 11-hydroxy-delta-9-tetrahydrocannabinol, 11-nor-9-carboxydelta-9-tetrahydrocannabinol), cocaine, benzoylecgonine, ecgonine methyl ester, cocaethylene, ketamine, and the opiates (e.g. heroin, 6-monoacetylmorphine, morphine, codeine, methadone and dihydrocodeine), or metabolites thereof. Non-limiting non-illicit substances of abuse include alcohol, nicotine, prescription medicine or over the counter medicine taken for non-medical reasons, a substance taken for a medical effect, wherein the consumption has become excessive or inappropriate (e.g. pain medications such as opiates, sleep aids, anti-anxiety medication, methylphenidate, erectile-dysfunction medications), and the like, or metabolites thereof.

In some embodiments, the analyte is a medicament or a component or metabolite thereof. A wide variety of medicaments are suitable analytes, including, but not limited to, cancer therapies, vaccines, analgesics, antipsychotics, antibiotics, anticoagulants, antidepressants, antivirals, sedatives, antidiabetics, contraceptives, immunosuppressants, antifungals, antihelmintics, stimulants, biological response modifiers, non-steroidal anti-inflammatory drugs (NSAIDs), corticosteroids, disease-modifying anti-rheumatic drugs (DMARDs), anabolic steroids, antacids, antiarrhythmics, thrombolytics, anticonvulsants, antidiarrheals, antiemetics, antihistamines, antihypertensives, anti-inflammatories, antineoplastics, antipyretics, barbiturates, β-blockers, bronchodilators, cough suppressants, cytotoxics, decongestants, diuretics, expectorants, hormones, laxatives, muscle relaxants, vasodilators, sedatives, vitamins, and metabolites thereof. Various examples of these medicaments are described herein and are well known in the art.

In some embodiments, the analyte is a poison, toxin, chemical warfare agent, or metabolite thereof. Suitable poisons, toxins and chemical warfare agents include, but are not limited to, including blister agents (e.g. cantharidin, furanocoumarin, sulfur mustards (e.g. 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl) sulfide, bis(2-chloroethylthio)methane, bis(2-chloroethylthiomethyl)ether, bis(2-chloroethylthioethyl)ether), nitrogen mustards (e.g. bis(2-chloroethyl)ethylamine, bis(2-chloroethyl)methylamine and tris(2-chloroethyl)amine) and phosgene oxime), arsenicals (e.g. ethyldichloroarsine, methyldichloroarsine, phenyldichloroarsine and 2-chlorovinyldichloroarsine) and urticants e.g. phosgene oxime), blood agents (e.g. cyanogen chloride, hydrogen cyanide and arsine), choking agents (e.g. chlorine, chloropicrin, diphosgene and phosgene), nerve agents (e.g. tabun, sarin, soman, cyclosarin, novichok agents, 2-(dimethylamino)ethyl-N,N-dimethylphosphoramidofluoridate (GV), (S)-(ethyl{[2-(diethylamino)ethyl]sulfanyl}(ethyl)phosphinate) (VE), O,O-diethyl-S-[2-(diethylamino)ethyl]phosphorothioate (VG), S-[2-(diethylamino)ethyl]-O-ethyl methylphosphonothioate (VM), ethyl(2-[bis(propan-2-yl)amino]ethyl sulfanyl)(methyl)phosphinate (VX), tetrodotoxin, saxitoxin and botulinum toxin), animal venom component (e.g. tetrodotoxin, saxitoxin or other component of spider, snake, scorpion, bee, wasp, ant, tick, conesnail, octopus, fish (e.g stonefish) and jellyfish venom), cyanide, arsenic, a component of Atropa Belladonna (deadly nightshade) such as a tropane alkaloid (e.g. atropine, scopolamine and hyoscyamine), a component of hemlock such as a piperidine alkaloid (e.g. coniine, N-methylconiine, conhydrine, pseudoconhydrine and gamma-coniceine), a curare alkaloid (e.g. tubocurarine), nicotine, caffeine, alcohol, quinine, atropine, strychnine, brucine, aflatoxin and metabolites thereof. In some embodiments, the analyte is a chemical warfare agent such as a blister agent (e.g. cantharidin, furanocoumarin, a sulfur mustard (e.g. 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl)sulfide, bis(2-chloroethylthio)methane, bis(2-chloroethylthiomethyl)ether or bis(2-chloroethylthioethyl)ether), a nitrogen mustard (e.g. bis(2-chloroethyl)ethylamine, bis(2-chloroethyl)methylamine or tris(2-chloroethyl)amine) or phosgene oxime), an arsenical (e.g. ethyldichloroarsine, methyldichloroarsine, phenyldichloroarsine or 2-chlorovinyldichloroarsine) or an urticant e.g. phosgene oxime), a blood agent (e.g. cyanogen chloride, hydrogen cyanide or arsine), a choking agent (e.g. chlorine, chloropicrin, diphosgene or phosgene), a nerve agent (e.g. tabun, sarin, soman, cyclosarin, a novichok agent, 2-(dimethylamino)ethyl-N,N-dimethylphosphoramidofluoridate (GV), (S)-(ethyl{[2-(diethylamino)ethyl]sulfanyl}(ethyl)phosphinate) (VE), O,O-diethyl-S-[2-(diethylamino)ethyl]phosphorothioate (VG), S-[2-(diethylamino)ethyl]-O-ethyl methylphosphonothioate (VM), ethyl({2-[bis(propan-2-yl)amino]ethyl}sulfanyl)(methyl)phosphinate (VX), tetrodotoxin, saxitoxin or botulinum toxin) or a metabolite thereof.

Examples of suitable analytes, diseases, disorders or conditions, or applications for which they are relevant and known lowest clinically relevant serum concentration ranges are provided in Table 1.

TABLE 1

| | | Lowest | | | |
|---|---|---|---|---|---|
| | | clinically | | | |
| | Relevant disease, | relevant | | | |
| | disorder or | concentration | | | |
| | condition, or | (where | | Molecular | |
| Analyte | application | available) | | weight | |
| Troponin or a subunit thereof, | Cardiac damage, | Less than 30 | | 23 kDa, 18 kDa | |
| such as troponin I, troponin C | myocardial | ng/L | | and 34 kDa, | |
| or troponin T | infarction, acute | | | respectively for | |
| | coronary syndrome | | | I, C and T | |
| | | | | subunits | |
| Cortisol (serum) | Addison's disease, | Less than 650 | | 362 | Da |
| | Cushing's disease, | nmol/L | | | |
| | adrenal and/or | | | | |
| | pituitary gland | | | | |
| | function, | | | | |
| | psychological stress | | | | |
| | (wellness | | | | |
| | applications) | | | | |
| Creatinine | Renal failure, | Less than 100 | | 113 | Da |
| | creatinine clearance | μmol/L | | | |
| | estimates | | | | |
| Dopamine | Parkinson's disease, | 0-30 | pg/mL | 153 | Da |
| | brain cancers, | | | | |
| | depression | | | | |
| Aminoglycosides (e.g. | Monitor dose of | 5-10 | mg/L | Varied ~300- | |
| kanamycin, gentamicin, | therapeutic for | | | 600 Da | |
| streptomycin) | bacterial infection | | | | |
| Anticonvulsants (e.g. | Monitor dose of | 0.02-12 | mg/L | Varied ~100 Da | |
| carbamazepine and clonazepam) | therapeutic for | | | | |
| | epilepsy | | | | |
| Hormones such as follicle | Assisted fertility, | Varied | | Varied ~200- | |
| stimulating hormone, luteinising | calcium levels, | | | 300 Da | |
| hormone, oxytocin, gonadotropin- | substance abuse | | | | |
| releasing hormone and | (doping) | | | | |
| testosterone | | | | | |
| Amylase | Pancreatitis, bile | Less than 100 | | 50 | kDa |
| | duct obstruction | U/L | | | |
| Creatinine kinase | Skeletal muscle | Less than 200 | | 80 | kDa |
| | damage, which may | U/L | | | |
| | be indicative of | | | | |
| | rhabdomyolysis, | | | | |
| | injury and/or drug | | | | |
| | side-effects (statins) | | | | |
| Lactate dehydrogenase | Hepatic damage | 119-229 | U/L | 140 | kDa |
| B-type natriuretic peptide | Cardiac failure | 100 | ng/L | 36 kDa (high | |
| (BNP) | | | | molecular | |
| | | | | weight form) or | |
| | | | | 3.5 kDa (low | |
| | | | | molecular | |
| | | | | weight form) | |
| NT-proBNP | Cardiac failure | 300 | ng/L | 8.5 | kDa |
| Angiotensin II converting | Essential | 8-100 | U/L | 60-170 | kDa |
| enzyme | hypertension | | | | |
| Cystatin C | Renal failure | 0.6-1 | mg/L | 13 | kDa |
| Stress hormones e.g. | Adrenal | 2-11 | pmol/L | ~4 | kDa |
| adrenocorticotropic hormone | insufficiency or | | | | |
| (ACTH) | overactivity | | | | |
| Inflammatory markers (e.g. | Bacterial or viral | Less than 10 | | Varied 120 kDa | |
| C-reactive protein (CRP), | infection, | mg/L (CRP) | | (CRP) | |
| TNFα, IL-6, ICAM1, TLR2, | autoimmune | | | | |
| TLR4, presepsin) | disorders, | | | | |
| | rheumatological | | | | |
| | disorders, sepsis | | | | |
| Inulin | Renal failure, | Varied | | Varied | |
| | creatinine clearance | (dependent on | | | |
| | estimates | amount | | | |
| | | administered) | | | |
| Illicit substances (e.g. | Drug abuse, | Varied | | Varied ~200- | |
| methamphetamine, amphetamine, | compliance | (dependent on | | 300 Da | |
| 3,4- | monitoring, | application e.g. | | | |
| methylenedioxymethamphetamine | rehabilitation, | rehabilitation | | | |
| (MDMA), N-ethyl-3,4- | screening | compared with | | | |
| methylenedioxyamphetamine | | screening or | | | |
| (MDEA), 3,4-methylenedioxy- | | drug abuse, | | | |
| amphetamine (MDA), | | and identity of | | | |
| cannabinoids (e.g. delta-9- | | substance) | | | |
| tetrahydrocannabinol, 11- | | | | | |
| hydroxy-delta-9- | | | | | |
| tetrahydrocannabinol, 11-nor-9- | | | | | |
| carboxydelta-9- | | | | | |
| tetrahydrocannabinol), cocaine, | | | | | |
| benzoylecgonine, ecgonine | | | | | |
| methyl ester, cocaethylene, | | | | | |
| ketamine, and the opiates (e.g. | | | | | |
| heroin, 6-monoacetylmorphine, | | | | | |
| morphine, codeine, methadone | | | | | |
| and dihydrocodeine)) | | | | | |
| Anticoagulants (e.g. warfarin and | Monitor dose of | Varied | | Varied | |
| heparin) | therapeutic for | | | | |
| | blood clotting | | | | |
| | disorders and | | | | |
| | diseases | | | | |
| Glycoproteins and glycans | Bacterial infection | Varied | | Varied ~10- | |
| | (i.e. bacterial | | | 20 kDa | |
| | endotoxins) | | | | |
| Cellular components and | Bacterial infection, | Varied | | Varied | |
| breakdown products | exosome detection, | | | | |
| | cancer, platelet | | | | |
| | detection | | | | |
| D-dimer | Pulmonary | 0.4 | mg/mL | 180 | kDa |
| | embolism | | | | |

| | | | |
|---|---|---|---|
| Oligonucleotides and | Bacterial infection, | Varied | Varied ~200- |
| polynucleotides (e.g. DNA, RNA | viral infection, | | 300 Da |
| and fragments thereof) | circulating tumour | | |
| | cell breakdown, | | |
| | solid tissue cancers | | |
| Chemical warfare agents (e.g. | Chemical warfare, | Varied | Varied |
| blister agents, blood agents, | environmental | | |
| choking agents and nerve agents) | contamination | | |

In some embodiments, the analyte is a metabolite of any one of the above exemplary analytes.

While the analyte preferably binds directly to the binding agent, the invention also contemplates detecting agents probative of the analyte of interest such as a specific binding pair member complementary to the analyte of interest, whose presence will be detected only when a particular analyte of interest is present in a sample. Thus, the agent probative of the analyte becomes the analyte that is detected.

In some embodiments, the microstructures are coated with a material that reduces absorption of analytes that are not of interest. Example materials include alkyl groups coated with BSA (bovine serum albumin), bifunctional polyethylene glycol (PEG) polymers, or the like. Such materials have the effect of reducing adsorption of non-specific analytes, which are effectively repelled from the microstructures.

When a coating is used to perform the detection, the coating can be adapted to change in appearance in response to a presence, absence, level or concentration of analytes, cause a change in appearance of surrounding tissue in response to a presence, absence, level or concentration of analytes or cause a change in appearance of analytes. For example, analytes could be labelled with a fluorophore, allowing the response of the fluorophores to be used to detect the presence of analytes.

Additionally and/or alternatively, the coating could also be adapted to dissolve. This could occur after a defined time period, so that detection is only performed once the time period has expired, in response to a presence, absence, level or concentration of analytes or upon breaching or penetration of the functional barrier. Thus, it will be appreciated that changes in appearance could be triggered by events, such as breaching or penetration of the functional barrier. For example, some microstructures might react to analytes, whilst others react to breaching of the barrier, so that successful application can be detected in addition to detection of analytes.

In one example, the microstructures are coated with a material that reduces absorption of analytes that are not of interest. Example materials include alkyl groups coated with BSA (bovine serum albumin), bifunctional polyethylene glycol (PEG) polymers, or the like. Such materials have the effect of reducing adsorption of non-specific analytes, which are effectively repelled from the microstructures. In contrast, a coating including a binding agent, such as one or more probes can be designed to target specific analytes of interest, which are adapted to bind with or otherwise attach to the probes through an appropriate mechanism.

It will be appreciated that multiple coatings could be used in conjunction, for example, to repel non-specific analytes whilst binding to probes allowing specific analytes of interest to be selectively captured, whilst non-specific analytes remain uncaptured.

The coating and/or the patch mechanical properties can also be generally selected to reduce non-specific protein binding, whilst simultaneously increasing the specific capture of specific proteins, such as AO-IgG (anti-ovalbumin IgG), or other antibodies, such as anti-influenza-IgG, or the like. In one example, the coating includes chains of polyethylene glycol (PEG) that repel non-specific protein binding. By using PEG chains terminating in carboxylic acid groups, this facilitates attachment of Protein A or Protein G via standard carbodiimide chemistry. AO-IgG capture probes form strong near-covalent bonds with Protein A/G in the optimal orientation for target binding.

The microstructures can be coated using a variety of techniques, including dip coating, spray coating, deposition coating, or the like. In one example, a coating solution is applied to the microstructures and allowed to dry in situ, optionally using a gas jet. To optimise coating, properties of the coating can be controlled through the addition of one or more other agents such as a viscosity enhancer, a detergent or other surfactant, and an adjuvant. These ingredients can be provided in a range of different concentrations. For example, the viscosity enhancer or surfactant can form between 0% and 90% of the coating solution.

A range of different viscosity enhancers can be used and examples include methylcellulose, carboxymethylcellulose (CMC), gelatin, agar, and agarose and any other viscosity modifying agents. The solution typically has a viscosity of between 10⁻³ Pa·s and 10⁻¹ Pa·s. In one example, using a coating solution containing 1-2% methylcellulose, which results in suitable uniform coatings, resulting in a viscosity within the range 0.011 (1%)-0.055 (2%) Pa·s.

Similarly, a range of different surfactants can be used to modify the surface tension of the coating solution, such as any detergent or any suitable agent that decreases surface tension, and that is biocompatible at a low concentration. The solution properties are also typically controlled through the addition of one or more other agents such as a viscosity enhancer, a detergent, other surfactant, or anything other suitable material. These ingredients can be provided in a range of different concentrations. For example, the viscosity enhancer or surfactant can form between 0% and 90% of the coating solution.

As an alternative to using a coating technique, reagents can alternatively be embedded within the patches. Thus, for example, in the case of moulded patches manufactured using a polymer material, the reagent can be introduced into the mould together with the polymer material so that the reagent is distributed throughout the structures. In this example, the polymer can be arranged so that pores form within the structures during the curing process.

Using affinity surface coatings on each structure also allows a reduction of non-specific adsorption of ISF and/or blood components whilst facilitating specific extraction of the molecular targets of interest.

It will be appreciated that microstructures could be differentially coated, for example by coating different microstructures with different coatings, and/or by coating different parts of the microstructures with different coatings. For example, at least some microstructures could be porous with an internal coating, whilst some microstructures might be only partially coated. In one example, the coating can be selectively applied to allow different analytes to be detected at different depths, so that for example a different coating is used for part of the microstructure that enters the dermis as opposed to the viable epidermis. This could also be used to allow for detection of different analytes, or different concentrations of the same analyte. Additionally, at least some microstructures could remain uncoated, for example, to allow these to be used as a control. It will also be appreciated that multiple coatings could be provided. For example, an outer coating could be provided that gives mechanical strength during insertion, and which dissolves once in-situ, allowing an underlying functional coating to be exposed, for example to allow analytes to be detected.

In another example, the microstructures can be coated with a selectively dissolvable coating. The coating could be adapted to dissolve after a defined time period, such as after the microstructures have been present within the subject for a set length of time, in response to the presence of one or more reagents in the subject, or in response application of a stimulatory signal, such as an electrical signal, optical signal or the like. Dissolving of the coating can be used in order to trigger a measurement process, for example by exposing a binding agent, or other functional feature, so that analytes are only detected once the coating has dissolved.

In one example, the coating can be used to provide mechanical properties. For example, the coating can provide a physical structure that can be used to facilitate penetration of the barrier, for example by providing a microstructure with a smooth tapered outer profile. The coating can strengthen the microstructures, to prevent microstructures breaking, fracturing, buckling or otherwise being damaged during insertion, or could be used to help anchor the microstructures in the subject. The coating could change the shape of the projection, allowing this to be used to anchor the projections. For example, the coating could include hydrogels, which expand upon exposure to moisture, so that the size of the microstructure and coating increases upon insertion into the subject, thereby it harder to remove the microstructure.

The coating can also be used to modify surface properties of the microstructures, for example to increase or decrease hydrophilicity, increase or decrease hydrophobicity and/or minimize biofouling. The coating can also be used to attract or repel at least one substance, such as analytes, cells, fluids, or the like. The coating could also dissolve to expose a microstructure, a further coating or material, allowing this to be used to control the detection process. For example, a time release coating could be used to enable a measurement to be performed a set time after the patch has been applied. This could also be used to provide stimulation to the subject, for example by releasing a treatment or therapeutic material, or the like.

Thus, in one example, at least some of the microstructures attract at least one substance to the microstructures and/or repel at least one substance from the microstructures. The nature of the substance will vary depending on the preferred implementation and may include one or more analytes, or may include other substances containing analytes, such as ISF, blood or the like. This can be used to attract or repel analytes, for example attracting analytes of interest, allowing these to be concentrated and/or sensed, or repelling analytes that are not of interest.

The ability to repel substances can also assist with preventing biofouling. For example, the microstructures could contain a material, or include a coating, such as Polyethylene glycol (PEG), which generally repels substances from the surface of the microstructure. Reduction in biofouling could also be achieved based on a choice of microstructure material, surface coatings that release to expose a sensing surface when sensing is to be performed, permeable coatings, releasable coatings that interfere with biofouling material. Physical mechanisms can also be used, including retracting the microstructures when sensing is not being performed. In this instance, whilst removed from the body, the microstructures could be cleaned, recoated or reconditioned, thereby mitigating or removing any biofouling.

The nature of the coating and the manner in which this is applied will vary depending on the preferred implementation and techniques such as dip coating, spray coating, jet coating or the like, could be used, as described above. The thickness of the coating will also vary depending on the circumstances and the intend functionality provided by the coating. For example, if the coating is used to provide mechanical strength, or contains a payload material to be delivered to the subject, a thicker coating could be used, whereas if the coating is used for sensing other applications, a thinner coating might be required.

In another example, the microstructures can be coated with a selectively dissolvable coating. The coating could be adapted to dissolve after a defined time period, such as after the microstructures have been present within the subject for a set length of time, in response to the presence, absence, level or concentration of one or more analytes in the subject, upon breaching or penetration of the functional barrier, or in response application of a stimulatory signal, such as an electrical signal, optical signal or the like. In a further example, dissolving of the coating could be detected, for example through a change in optical or electrical properties, with the measurement being performed after the coating has dissolved. Thus, dissolving of the coating could be detected based on a change in a response signal.

The substrate can include a plurality of microstructures, with different microstructures being differentially responsive, for example to allow different microstructures to respond to different analytes or different concentrations of analytes and/or breaching or penetration of the barrier. In this case, different microstructures are typically provided in different regions of the substrate so that a visual appearance of different parts of the substrate can indicate successful breaching or penetration, the presence or absence of different analytes or different concentrations of the same analyte. This allows more complex detection than the simple binary detection of the presence or absence of a single analyte. For example, once a detection process has been performed, a user could identify any patterns or regions defined on the substrate, and use the appearance of each region or pattern to determine an associated meaning.

By way of example, the patch might divided into four regions, each corresponding to a respective analyte. Each region can be graduated, so that the degree of graduation indicates a concentration. In this instance, examining the degree of graduation of each region allows concentration of each of the four analytes to be determined.

In a further example, interpretation of the detection can be facilitated by having the appearance of the substrate include a pattern to indicate a presence, absence, level or concentration of analytes. For example, individual microstructures could act as individual pixels, allowing a pattern to be created based on changes in appearance of different microstructures. In one example, this can be used to display an alphanumeric pattern or iconic pattern, for example showing a tick or cross depending on whether or not analytes are present.

The nature of the substrate and/or microstructures will vary depending upon the preferred implementation, although substrate and microstructures are typically at least partially transparent to electromagnetic radiation. For example, substrate and/or microstructures could be made from or contain fabric, woven fabric, electronic fabric, natural fibres, silk, organic materials, natural composite materials, artificial composite materials, ceramics, stainless steel, metals, such as titanium or platinum, polymers, such as rigid or a semi-rigid plastics, including doped polymers, silicon or other semiconductors, including doped semiconductors, organosilicates, gold, silver, carbon, carbon nano materials, or the like. The substrate and microstructures could be made from similar and/or dissimilar materials, and could be integrally formed, or made separately and bonded together. Microstructures can also be provided on one or more substrates, so for example, signals could be measured or applied between microstructures on separate substrates.

It will be appreciated that the particular material used will depend on the intended application, so for example different materials will be used if the microstructure needs to be transparent to visible radiation only as opposed to visible and infra-red radiation.

The substrate and microstructures can be manufactured using any suitable technique. For example, in the case of silicon-based structures, this could be performed using etching techniques. Polymer or plastic structures could be manufactured using additive manufacturing, such as 3D printing, or moulding. In one particular example, a mould is filled with a suitable filling material, such as a solution containing a material such as an active compound and/or sugar-based excipient, such as carboxy-methylcellulose (CMC), or one or more polymers, or the like, which is then cured and removed. It will also be appreciated that the filling material may include any required probes, reagents, or the like that are to be contained within the structures, as will be discussed in more detail below. Photosensitive polymers might be used, such as photoresists, including SU8 or polyimides, for direct patterning of electrodes on the substrate or to make microstructures. Successive layers of photosensitive resists, polymers, metals, or the like, can be deposited and/or selectively removed to produce bespoke 3D microstructure geometries.

In one example, the substrate could be at least partially flexible in order to allow the substrate to conform to the shape of a subject and thereby ensure penetration of the microstructures into the viable epidermis and/or dermis, or other functional barrier. In this example, the substrate could potentially be a textile or fabric, with electrodes and circuitry woven in, or multiple substrates could be mounted on a flexible backing, to provide a segmented substrate arrangement. Alternatively, the substrate could be shaped to conform to a shape of the subject, so that the substrate is rigid but nevertheless ensures penetration of the microstructures.

In preferred examples, the substrate and microstructures are formed from one or more of metal, polymer or silicon.

The microstructures could have a range of different shapes and could include ridges, needles, plates, blades, or similar. In this regard, the terms plates and blades are used interchangeably to refer to microstructures having a width that is of a similar order of magnitude in size to the length, but which are significantly thinner. The microstructures can be tapered to facilitate insertion into the subject, and can have different cross-sectional shapes, for example depending on the intended use. The microstructures typically have a rounded rectangular shape and may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode.

Other example shapes include circular, rectangular, cruciform shapes, square, rounded square, rounded rectangular, ellipsoidal, or the like, which can allow for increased surface area, which is useful when coating microstructures to maximise the coating volume and hence the amount of payload delivered per microstructure, although it will be appreciated that a range of other shapes could be used. Microstructures can have a rough or smooth surface, or may include surface features, such as pores, raised portions, serrations, or the like, which can increase surface area and/or assist in penetrating or engaging tissue, to thereby anchor the microstructures within the subject. This can also assist in reducing biofouling, for example by prohibiting the adherence and hence build-up of biofilms. The microstructures might also be hollow or porous and can include an internal structure, such as holes or similar, in which case the cross sectional shape could also be at least partially hollow. In particular embodiments, the microstructures are porous, which may increase the effective surface area of the microstructure. The pores may be of any suitable size to allow an analyte of interest to enter the pores, but exclude one or more other analytes or substances, and thus, will depend on the size of the analyte of interest. In some embodiments, the pores may be less than about 10 μm in diameter, preferably less than about 1 in diameter.

In one example, the microstructures have a rounded rectangular shape when viewed in cross section through a plane extending laterally through the microstructures and parallel to but offset from the substrate. The microstructures may include shape changes along a length of the microstructure. For example, microstructures could include a shoulder that is configured to abut against the stratum corneum to control a depth of penetration and/or a shaft extending to the tip, with the shaft being configured to control a position of the tip in the subject.

Different microstructures could be provided on a common substrate, for example providing different shapes of microstructure to achieve different functions. In one example, this could include performing different types of measurement. In other examples, microstructures could be provided on different substrates, for example, allowing sensing to be performed via microstructures on one patch and delivery of therapy to be performed via microstructures on a different patch. In this example, this could allow a therapy patch to be replaced once exhausted, whilst a sensing patch could remain in situ. Additionally, measurements could be performed between patches, for example, performing whole of body impedance measurements between patches provided at different locations on a subject.

Additionally and/or alternatively anchor microstructures could be provided, which can be used to anchor the substrate to the subject. In this regard, anchor microstructures would typically have a greater length than that of the microstructures, which can help retain the substrate in position on the subject and ensure that the substrate does not move during the measurements or is not being inadvertently removed. Anchor microstructures can include anchoring structures, such as raised portions, which can assist with engaging the tissue, and these could be formed by a shape of the microstructure and/or a shape of a coating. Additionally, the coating could include a hydrogel or other similar material, which expands upon expose to moisture within the subject, or an applied stimulation, thereby further facilitating engagement with the subject. Similarly the microstructure could undergo a shape change, such as swelling either in response to exposure to substances, such as water or moisture within the subject, or in response to an applied stimulation. When applied to skin, the anchor microstructures can enter the dermis, and hence are longer than other microstructures, to help retain the substrate in place, although it will be appreciated that this is not essential and will depend upon the preferred implementation. In other examples the anchor microstructures are rougher than other microstructures, have a higher surface friction than other microstructures, are blunter than other microstructures or are fatter than other microstructures.

In a further example, at least part of the substrate could be coated with an adhesive coating in order to allow the substrate and hence patch, to adhere to the subject.

As previously mentioned, when applied to skin, the microstructures typically enter the viable epidermis and in one example, do not enter the dermis, although in other examples, may enter the dermis. But this is not essential, and for some applications, it may be necessary for the microstructures to enter the dermis, for example projecting shortly through the viable epidermis/dermis boundary or entering into the dermis a significant distance, largely depending on the nature of the sensing being performed. In one example, for skin, the microstructures have a length that is at least one of less than 2500 μm, less than 1000 μm, less than 750 μm, less than 600 μm, less than 500 μm, less than 400 μm, less than 300 μm, less than 250 μm, greater than 100 μm, greater than 50 μm and greater than 10 μm, but it will be appreciated that other lengths could be used. More generally, when applied to a functional barrier, the microstructures typically have a length greater than the thickness of the functional barrier, at least 10% greater than the thickness of the functional barrier, at least 20% greater than the thickness of the functional barrier, at least 50% greater than the thickness of the functional barrier, at least 75% greater than the thickness of the functional barrier and at least 100% greater than the thickness of the functional barrier.

In another example, the microstructures have a length that is no more than 2000% greater than the thickness of the functional barrier, no more than 1000% greater than the thickness of the functional barrier, no more than 500% greater than the thickness of the functional barrier, no more than 100% greater than the thickness of the functional barrier, no more than 75% greater than the thickness of the functional barrier or no more than 50% greater than the thickness of the functional barrier. This can avoid deep penetration of underlying layers within the body, which can in turn be undesirable, and it will be appreciated that the length of the microstructures used will vary depending on the intended use, and in particular the nature of the barrier to be breached, and/or signals to be applied or measured. The length of the microstructures can also be uneven, for example, allowing a blade to be taller at one end than another, which can facilitate penetration of the subject or functional barrier.

Similarly, the microstructures can have different widths depending on the preferred implementation. Typically, the widths are at least one of less than 25% of the length, less than 20% of the length, less than 15% of the length, less than 10% of the length, or less than 5% of the length. Thus, for example, when applied to the skin, the microstructures could have a width of less than 50 μm, less than 40 μm, less than 30 μm, less than 20 μm or less than 10 μm. However, alternatively, the microstructures could include blades, and could be wider than the length of the microstructures. In some example, the microstructures could have a width of less than 50000 μm, less than 40000 μm, less than 30000 μm, less than 20000 μm, less than 10000 μm, less than 5000 μm, less than 2500 μm, less than 1000 μm, less than 500 μm or less than 100 μm. In blade examples, it is also feasible to use microstructures having a width substantially up to the width of the substrate.

In general the thickness of the microstructures is significantly lower in order to facilitate penetration and is typically less than 1000 μm, less than 500 μm, less than 200 μm, less than 100 μm, less than 50 μm, less than 20 μm, less than 10 μm, at least 1 μm, at least 0.5 μm or at least 0.1 μm. In general the thickness of the microstructure is governed by mechanical requirements, and in particular the need to ensure the microstructure does not break, fracture or deform upon penetration. However, this issue can be mitigated through the use of a coating that adds additional mechanical strength to the microstructures.

In one specific example, for epidermal sensing, the microstructures have a length that is less than 300 μm, greater than 50 μm, greater than 100 μm and about 150 μm, and, a width that is greater than or about equal to a length of the microstructure, and is typically less than 300 μm, greater than 50 μm and about 150 μm. In another example, for dermal sensing, the microstructures have a length that is less than 450 μm, greater than 100 μm, and about 250 μm, and, a width that is greater than or about equal to a length of the microstructure, and at least of a similar order of magnitude to the length, and is typically less than 450 μm, greater than 100 μm, and about 250 μm. In other examples, longer microstructures could be used, so for example for hyperdermal sensing, the microstructures would be of a greater length. The microstructures typically have a thickness that is less than the width, significantly less than the width and of an order of magnitude smaller than the width. In one example, the thickness is less than 50 μm, greater than 10 μm, and about 25 μm, whilst the microstructure typically includes a flared base for additional strength, and hence includes a base thickness proximate the substrate that is about three times the thickness, and typically is less than 150 μm, greater than 30 μm and about 75 μm. The microstructures typically have a tip has a length that is less than 50% of a length of the microstructure, at least 10% of a length of the microstructure and more typically about 30% of a length of the microstructure. The tip further has a sharpness that is at least 0.1 μm, less than 5 μm and typically about 1 μm.

In general the thickness of the microstructures is significantly lower in order to facilitate penetration and is typically less than 1000 μm, less than 500 μm, less than 200 μm, less than 100 μm, less than 50 μm, less than 20 μm, less than 10 μm, at least 1 μm, at least 0.5 μm or at least 0.1 μm. In general the thickness of the microstructure is governed by mechanical requirements, and in particular the need to ensure the microstructure does not break, fracture or deform upon penetration. However, this issue can be mitigated through the use of a coating that adds additional mechanical strength to the microstructures.

In one example, the microstructures have a relatively low density, such as less than 10000 per cm², such as less than 1000 per cm², less than 500 per cm², less than 100 per cm², less than 10 per cm² or even less than 5 per cm². The use of relatively a low density facilitates penetration of the microstructures through the stratum corneum and in particular avoids the issues associated with penetration of the skin by high density arrays, which in turn can lead to the need for high powered actuators in order for the arrays to be correctly applied. However, this is not essential, and higher density microstructure arrangements could be used, including less than 50,000 microstructures per cm², less than 30,000 microstructures per cm², or the like. As a result, the microstructures typically have a spacing that is less than 20 mm, less than 10 mm, less than 1 mm, less than 0.1 mm or less than 10 μm. It should be noted that in some circumstances, microstructures are arranged in pairs, with the microstructures in each pair having a small spacing, such as less than 10 μm, whilst the pairs have a great spacing, such as more than 1 mm, in order to ensure a low overall density is maintained. However, it will be appreciated that this is not essential, and higher densities could be used in some circumstances.

In one specific example, the microstructures have a density that is less than 5000 per cm², greater than 100 per cm², and about 600 per cm², leading to a spacing of less than 1 mm, more than 10 μm, and about 0.5 mm, 0.2 mm or 0.1 mm.

In one example, when optical sensing is performed, the connections in the substrate include waveguides, or other electromagnetically conductive paths, such as optical fibre, which extend through the microstructures to one or more ports in the microstructure, to allow electromagnetic radiation to be emitted from or received via the ports. In one example, this is achieved by having the microstructure made from, or contain, polymer, or another similar material, which is at least partially transparent to the frequency of electromagnetic radiation being applied or received, which could include visible radiation, ultra-violet radiation, infra-red radiation, or the like, depending on the preferred application.

In one example, an at least partially electromagnetically transparent core can be surrounded by an outer electromagnetically opaque layer, with ports extending through the opaque layer, to allow electromagnetic radiation to be emitted or received via the ports. In this example, it will be appreciated that appropriate positioning of the ports, allows radiation to be delivered or received in a targeted manner, for example allowing this to be directed into a particular depth within the viable epidermis, or elsewhere. In one example, the transparent core could be made from a waveguide, such as a fibre optic cable, or part thereof. For example, the outer layer and/or reflective layer could be removed, allowing the transparent core of the microstructure to be made of the fibre optic core. In a further example, the microstructures include electromagnetically reflective layers to allow electromagnetic radiation to be conducted to and from designated ports.

In one example, the microstructures can be configured to deliver stimulation including any one or more of biochemical, chemical, mechanical, magnetic, thermal, electrical, electromagnetic or optical. This could be achieved for example, by releasing suitable materials into the subject, as previously described, or by applying external signals.

In one example, the one or more microstructure electrodes interact with one or more analytes of interest such that a response is dependent on a presence, absence, level or concentration of analytes of interest.

In one example, at least some of the microstructures are arranged in groups, with microstructures within the group can have a specific configuration to allow particular analytes to be detected. Using groups of microstructures in this manner allows different regions of the substrate to present a visual appearance relevant to a particular detection, whilst ensuring the effect is sufficient to be visually observed. For example, different regions of the substrate could include microstructures responsive to different analytes, so multiple analytes can be detected using a single patch. Alternatively, different regions of a patch could be responsive to different concentrations of an analyte, so that responsive regions can be used to identify concentrations of analytes.

In one specific example, microstructures are angularly offset, and/or orthogonally arranged, and additionally and/or alternatively, microstructures can be arranged in rows, with the microstructures in one row are orthogonally arranged or angularly offset relative to microstructures in other rows. Thus, in the case of plate microstructures, at least some microstructures extend in different and optionally orthogonal directions. This distributes stresses associated with insertion of the patch in different directions, and also acts to reduce sideways slippage of the patch by ensuring plates at least partially face a direction of any lateral force. Reducing slippage either during or post insertion helps reduce discomfort, erythema, or the like, and can assist in making the patch comfortable to wear for prolonged periods.

In one example, the system includes an actuator configured to apply force to the substrate, which in one example is used to help the microstructures to breach the barrier. The actuator could additionally and/or alternatively be used for other purposes.

For example, movement of the microstructures could be used to sense tissue mechanical properties. For example, a response of the actuator, such as an amount of current required to induce movement of the microstructures, could be used sense mechanical properties, such as a degree of elasticity, or the like, which can in turn be indicative of health issues, such as diseases or similar. This could also be used in conjunction with mechanical response signals, for example measuring a stress or strain on the microstructures using a suitable sensing modality, allowing the transmission of actuator movements to be monitored. Other external mechanical stimulus could also be used, such providing a ring or other structure around the patch, which generates pressure waves within the tissue, allowing the responses to be measured.

The actuator can be used to provide mechanical stimulation, for example to trigger a biological response, such as inflammation, or to attract or repel substances. Additionally, physical movement can be used to release material from a coating on at least some microstructures, or could be used to disrupt, dissolve, dislodge or otherwise release a coating on at least some microstructures. This can be used to trigger a measurement process, for example, releasing a coating or material to trigger a reaction with analytes, allowing the analytes to be detected.

The actuator can also be used to cause the microstructures to penetrate the barrier, or retract the microstructures from the barrier and/or the subject. In one example, this allows the microstructures to be inserted and removed from the subject as needed, so that microstructures can be removed when measurements are not being performed. This can be used to comfort, to reduce the chance of infection, reduce biofouling, or the like.

As the microstructures are provided in a low-density configuration, the force required is typically minimal, in which case this could be achieved utilising an actuator that provides a small force, such as piezoelectric actuator, or a mechanical actuator, such as an offset motor, vibratory motor, or the like. In one particular example, this is achieved using a vibratory, periodic or repeated force, which can assist with penetration, for example by agitating the microstructures to overcome the elasticity of the stratum corneum. However, this is not essential and single continuous or instantaneous forces could be used.

Other actuators could however be used, including any one or more of an electric actuator, a magnetic actuator, a polymeric actuator, a fabric or woven actuator, a pneumatic actuator, a thermal actuator, a hydraulic actuator, a chemical actuator, or the like. For example, a chemical or biochemical reaction, including exposure to air, light, water or other substance, could trigger exothermic release of energy, which can be used for to provide a mechanical impulse to urge the substrate and hence microstructures into the subject. It will also be appreciated that actuation could also be achieved manually, by applying a force to the patch, or by using a strap or similar to urge the patch against the subject.

In one specific example, this is achieved using a biasing force, for example provided by a spring or electromagnetic actuator, together with a vibratory, periodic or repeated force, which can assist with penetration, for example by agitating the microstructures to overcome the elasticity of the stratum corneum and/or reduce friction for penetrating the epidermis and/or dermis, as well as to reduce the force required to pierce a barrier. This reduces the overall force required to penetrate the stratum corneum. However, this is not essential and single continuous or instantaneous forces could be used.

The frequency of vibration used will vary depending upon the preferred implementation and potentially the type of skin to which the microstructures are applied, and could include any one or more of at least 0.01 Hz, 0.1 Hz, 1 Hz, at least 10 Hz, at least 50 Hz, at least 100 Hz, at least 1 kHz, at least 1 kHz, or at least 100 kHz and potentially up to several MHz. In one example, a varying frequency could be used. The frequency could vary depending on a wide range of factors, such as a time of application, and in particular the length of time for which the application process has been performed, the depth or degree of penetration, a degree of resistance to insertion, or the like. In one example, the system uses response signals measured via the microstructures in order to detect when the barrier has been breached, such as when the microstructures have penetrated the stratum corneum. Thus, the frequency could be continuously varied, either increasing or decreasing, until successful penetration is achieved, or depending on a depth of penetration, which can be detected using response signals, at which point the actuator can be deactivated. In another example, the frequency starts high and progressively reduces as the microstructures penetrate the barrier, and in particular the stratum corneum.

In another example, the magnitude of the applied force can also be controlled. The force used will vary depending on a range of factors, such as the structure of the patch, the manner in which the patch is applied, the location of application, the depth of penetration, or the like. For example, patches with large numbers of microstructures typically require an overall higher force in order to ensure penetration, although for minimal numbers of microstructures, such as 10 or so, a larger force may be required to account for damping or loss from the substrate/skin. Similarly, the force required to penetrate the stratum corneum, would typically be higher than that required to penetrate the buccal mucosa. In one example, the applied force could be any one or more of at least 0.1 μN, at least 1 μN, at least 5 μN, at least 10 μN, at least 20 μN, at least 50 μN, at least 100 μN, at least 500 μN, at least 1000 μN, at least 10 mN, or at least 100 mN, per microstructure and/or collectively. For example, if there are 1000 microstructures, the force could be 100 mN in total, or 100 mN per projection, leading to an overall 100 N force.

Again, the force could vary, either increasing or decreasing, depending on a time of application, a depth or degree of penetration, which could be determined by examining a change in insertion resistance, or the like. In one specific example, the force is progressively increased until a point of penetration, at which point the force decreases.

As mentioned above, the force could be applied as a single continuous or instantaneous force. However, more typically the force is periodic. In this instance the nature of the periodic motion could vary, this could for example, have any waveform, including square waves, sine waves, triangular waves, variable waveforms, or the like. In this case, the force could be an absolute magnitude, or could be a peak-to-peak or Root Mean Square (RMS) force.

Similarly, a magnitude of movement of the microstructures can also be controlled. The degree of magnitude will depend on factors, such as the length of the microstructures and the degree of penetration required. The magnitude could include any one or more of greater than 0.001 times a length of the microstructure, greater than 0.01 times a length of the microstructure, greater than 0.1 times a length of the microstructure, greater than a length of the microstructure, greater than 10 times a length of the microstructure, greater than 100 times a length of the microstructure or greater than 1000 times a length of the microstructure. The magnitude may also vary, either increasing or decreasing, depending a time of application, a depth of penetration, a degree of penetration or an insertion resistance. Again, the magnitude may increase until a point of penetration and then decrease after a point of penetration.

In the above example, the system can be configured to detect aspects of the insertion process. In one example, this can be achieved by monitoring the actuator, for example, monitoring the current required by the actuator to achieve a specific movement, which can in turn be used to detect, a depth of penetration, a degree of penetration an insertion resistance, or the like, with this then being used to control the actuator.

The actuator can also be used to apply mechanical stimulation, which could be used for a variety of purposes. For example, the actuator can be configured to physically disrupt or dislodge a coating on the microstructures, physically stimulate the subject, cause the microstructures to penetrate the barrier, retract the microstructures from the barrier or retract the microstructures from the subject.

The actuator is typically operatively coupled to the substrate, which could be achieved using any suitable mechanism, such a mechanical or electromagnetic engagement, or the like, depending on the nature of the actuator.

In one specific example, the actuator includes a spring or electromagnetic actuator to provide a constant bias, and at least one of a piezoelectric actuator and vibratory motor to apply a vibratory force. The vibratory force is applied at a frequency that is at least 10 Hz, less than 1 kHz and about 100-200 Hz. The continuous force is typically greater than 1 N, less than 10 N and about 5 N, whilst the vibratory force is at least 1 mN, less than 1000 mN and about 200 mN. The actuator is typically configured to cause movement of the microstructures that is at least 10 μm, less than 300 μm and about 50 μm to 100 μm.

In one example, the system is configured to perform measurements over a time period, such as a few hours, days, weeks, or similar. To achieve this, the microstructures can be configured to remain in the subject during the time period, or alternatively could be removed when measurements are not being performed. In one example, the actuator can be configured to trigger insertion of the microstructures into the skin and also allow for removal of the microstructures once the measurements have been performed. The microstructures can then be inserted and retracted as needed, to enable measurements to be performed over a prolonged period of time, without ongoing penetration of the skin. However, this is not essential and alternatively short term measurements can be performed, in which case the time period can be less than 0.01 seconds, less than 0.1 seconds, less than 1 second or less than 10 seconds. It will be appreciated that other intermediate time frames could also be used.

The above described examples have focused on, but are not limited to visual inspection of the patch. Such visual inspection is particularly beneficial as this allows the detection process to be performed without requiring additional equipment. However, in some circumstances, the use of aids can facilitate detection of changes in appearance.

For example, the appearance could be viewable upon exposure to electromagnetic radiation having a defined wavelength range, such as infrared radiation, ultraviolet radiation or visible radiation. Similarly a specific intensity or polarisation might be required, in which case a radiation source might be provided to expose the patch to electromagnetic radiation of the required wavelengths.

Similarly, in one example, a change in appearance could be detectable using an imaging device, either to detect non-visible changes in appearance, such as changes in infrared responsiveness, or detect changes that cannot otherwise be perceived. In this case, the system can include one or more electronic processing devices that receive an image of the substrate from an imaging device and analyse the image to determine an indicator indicative of a presence, absence, level or concentration of analytes.

In one example, this could be achieved using a customised measuring device, although in another example, this could be achieved using a client device, such as a smartphone, tablet or similar, optionally used in conjunction with a suitable illumination source, such as an in-built camera flash, or other external source. Furthermore, for ease of illustration the remaining description will refer to a processing device, but it will be appreciated that multiple processing devices could be used, with processing distributed between the devices as needed, and that reference to the singular encompasses the plural arrangement and vice versa.

It will be appreciated that in one example, the processing devices can control the measurement process by controlling a radiation source to selectively expose the substrate to electromagnetic radiation and control the imaging device to capture an image in response to exposure of the substrate to electromagnetic radiation. This could be performed concurrently with exposure of the substrate to electromagnetic radiation, or could be performed a defined time after exposure of the substrate to electromagnetic radiation, for example to detect fluorescence or similar.

Once detection has been performed, the results of the detection can be analysed and used in order to display an indicator, such as an indication of a presence, absence, level or concentration of one or more analytes. This could involve interpreting the appearance of the patch, and specifically identifying any patterns or regions, and using the appearance of each region to determine an associated meaning. Additionally and/or alternatively, further interpretation could be performed, for example, to derive a health status indicator.

For example, once measurements have been performed, the one or more electronic processing devices analyse the measured response to determine at least one metric, which can then be used to determine an indicator which is at least partially indicative of a health status associated with the subject. For example, the system could be configured detect analyte concentrations, with the concentration being a metric. The metric can then be used to derive indicators, such as a health status, which could include the presence, absence, degree or prognosis of one or more medical conditions, a prognosis associated with a medical condition, a presence, absence, level or concentration of a biomarker, a presence, absence, level or concentration of an analyte, a presence, absence or grade of cancer, fluid levels in the subject, blood oxygenation, a tissue inflammation state, bioelectric activity, such as nerve, brain, muscle or heart activity or a range of other health states.

The manner in which this is performed will vary depending upon the preferred implementation and could be achieved by monitoring changes in the values over time, and may involve comparison to values measured for reference subjects having known medical conditions. Additionally, and/or alternatively, the indicator could be indicative of measured parameters associated with the subject, such as measured concentrations of analytes or other biomarkers. In one example, the electronic processing devices could apply the metric to at least one computational model to determine the indicator, with the computational model embodying the relationship between a health status and the one or more metrics. In this instance, the computational model could be obtained by applying machine learning to reference metrics derived from subject data measured for one or more reference subjects having known health statuses. In this instance, the health status could include therapeutic requirements, the presence, absence, degree or severity of a medical condition, or could include one or more measures otherwise associated with a health status, such as measurements of the presence, absence, level or concentration of one or more analytes or measurements of other biomarkers.

The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail. In one example, this can include training a single model to determine the indicator using metrics from reference subjects with a combination of different health states, or the like, although this is not essential and other approaches could be used.

In one example, the system can include a transmitter that transmits subject data, metrics or measurement data, allowing these to be analysed remotely. In one particular example, the system includes a portable monitoring device that performs the measurements, with a separate processing system being provided that generate, analyse or store subject data derived from the measurement data. This can then be used to generate an indicator at least partially indicative of a health status associated with the subject.

In one example, the system includes a housing containing at least the sensor and one or more electronic processing devices, and optionally including other components, such as a signal generator, actuator, power supply, wireless transceiver, or the like. In one particular example, the housing provides reader functionality that can be used to interrogate the microstructures, and which can be provided in an integrated device, or could be provided remote to the substrate and engaged or provided in proximity with the substrate when readings are to be performed.

In the integrated configuration, the reader is typically mechanically connected/integrated with the patch during normal use, allowing measurements to be performed automatically. For example, continual monitoring could be performed, with a reading being performed every 1 second to daily or weekly, typically every 2 to 60 minutes, and more typically every 5 to 10 minutes. The timing of readings can vary depending on the nature of the measurement being performed and the particular circumstance. So for example, an athlete might wish to undergo more frequent monitoring while competing in an event, and then less frequent monitoring during post event recovery. Similarly, for a person undergoing medical monitoring, the frequency of monitoring may vary depending on the nature and/or severity of a condition. In one example, the frequency of monitoring can be selected based on user inputs and/or could be based on a defined user profile, or the like.

In the integrated arrangement, the reader can be connected to the patch using conventional resistance bridge circuitry, with analogue to digital conversion being used to perform measurements.

Alternatively, the reader can be separate, which allows the reader to be removed when not in use, allowing the user to wear a patch without any integrated electronics, making this less intrusive. This is particularly useful for applications, such as sports, geriatric and paediatric medicine, or the like, where the presence of a bulkier device could impact on activities. In this situation, the reader is typically brought into contact or proximity with the patch allowing readings to be performed on demand. It will be appreciated that this requires a user/person to drive the interrogation. However, the reader could include alert functionality to encourage interrogation.

Readings could be performed wirelessly, optionally using inductive coupling to both power the patch and perform the reading as will be described in more detail below, although alternatively, direct physical contact could alternatively be used. In this example, the microstructures and tissue form part of a resonant circuit with discrete inductance or capacitance, allowing the frequency to be used to determine the impedance and hence fluid levels, or analyte levels or concentrations. Additionally, and/or alternatively, ohmic contacts could be used, where the reader makes electrical contact with connectors on the patch.

In either case, some analysis and interpretation of the hydration signal or analyte level or concentration may be performed in the reader, optionally allowing an indicator to be displayed on the reader using an output, such as an LED indicator, LCD screen, or the like. Additionally, and/or alternatively, audible alarms may be provided, for example providing an indication in the event that the subject is under or over hydrated or has an analyte level or concentration outside an acceptable range. The reader can also incorporate wireless connectivity, such as Bluetooth, Wi-Fi or similar, allowing reading events to be triggered remotely and/or to allow data, such as impedance values, hydration or analyte level or concentration indicators, or the like to be transmitted to remote devices, such as a client device, computer system, or cloud based computing arrangement.

In use, the housing typically couples to the substrate, allowing the housing and substrate to be attached and detached as needed. In one example, this could be achieved utilising any appropriate mechanism, such as electromagnetic coupling, mechanical coupling, adhesive coupling, magnetic coupling, or the like. This allows the housing and in particular sensing equipment to only be connected to the substrate as needed. Thus, a substrate could be applied to and secured to a subject, with a sensing system only being attached to the substrate as measurements are to be performed. However, it will be appreciated that this is not essential, and alternatively the housing and substrate could be collectively secured to the subject for example using an adhesive patch, adhesive coating on the patch/substrate, strap, anchor microstructures, or the like. In a further example, the substrate could form part of the housing, so that the substrate and microstructures are integrated into the housing.

When the housing is configured to attach to the substrate, the housing typically includes connectors that operatively connect to substrate connectors on the substrate, to thereby communicate signals between the signal generator and/or sensor, and the microstructures. The nature of the connectors and connections will vary depending upon the preferred implementation and the nature of the signal, and could include conductive contact surfaces, that engage corresponding surfaces on the substrate, or could include wireless connections, such as tuned inductive coils, wireless communication antennas, or the like.

In one example, the system is configured to perform repeated measurements over a time period, such as a few hours, days, weeks, or similar. To achieve this, the microstructures can be configured to remain in the subject during the time period, or alternatively could be removed when measurements are not being performed. In one example, the actuator can be configured to trigger insertion of the microstructures into the skin and also allow for removal of the microstructures once the measurements have been performed. The microstructures can then be inserted and retracted as needed, to enable measurements to be performed over a prolonged period of time, without ongoing penetration of the skin. However, this is not essential and alternatively short term measurements can be performed, in which case the time period can be less than 0.01 seconds, less than 0.1 seconds, less than 1 second or less than 10 seconds. It will be appreciated that other intermediate time frames could also be used.

In one example, once measurements have been performed, the one or more electronic processing devices analyse the measured response signals to determine an indicator indicative of a health and/or physiological status of the subject.

In one example, this is achieved by deriving at least one metric, which can then be used to determine an indicator. For example, the system could be configured to perform impedance measurements, with the metric corresponding to an impedance parameter, such as an impedance at a particular frequency, a phase angle, or similar. The metric can then be used to derive indicators, such as an indication of fluid levels, such as extra or intra cellular fluid levels.

The manner in which this is performed will vary depending upon the preferred implementation. For example, the electronic processing devices could apply the metric to at least one computational model to determine the indicator, with the computational model embodying the relationship between a health status and the one or more metrics. In this instance, the computational model could be obtained by applying machine learning to reference metrics derived from subject data measured for one or more reference subjects having known health statuses. In this instance, the health status could be indicative of organ function, tissue function or cell function, could include the presence, absence, degree or severity of a medical condition, or could include one or more measures otherwise associated with a health status, such as measurements of the presence, absence, level or concentration of one or more analytes or measurements of other biomarkers.

The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail. In one example, this can include training a single model to determine the indicator using metrics from reference subjects with a combination of different health states, or the like, although this is not essential and other approaches could be used.

Measured signals can also be used in other manners. For example, changes in metrics over time can be used to track changes in a health state or medical condition for a subject. Measured signals can also be analysed in order to generate images or to perform mapping. For example, tomography could be used to establish a 2D or 3D image of a region of the subject based on impedance measurements or similar. The signals could also be used in contrast imaging, or the like.

In one example, the system can include a transmitter that transmits measured subject data, metrics or measurement data such as response signals or values derived from measured response signals, allowing these to be analysed remotely.

In one particular example, the system includes a wearable patch including the substrate and microstructures, and a monitoring device (also referred to as a “reader”) that performs the measurements. The monitoring device could be attached or integrally formed with the patch, for example mounting any required electronics on a rear side of the substrate. Alternatively, the reader could be brought into contact with the patch when a reading is to be performed. In either case, connections between the monitoring device could be conductive (ohmic) contacts, but alternatively could be indicative coupling, allowing the patch to be wirelessly interrogated and/or powered by the reader.

The monitoring device can be configured to cause a measurement to be performed and/or to at least partially process and/or analyse measurements. The monitoring device can control stimulation applied to at least one microstructure, for example by controlling the signal generator and/or switches as needed. This allows the monitoring device to selectively interrogate different microstructures, allowing different measurements to be performed, and/or allowing measurements to be performed at different locations. This also allows microstructures to be selectively stimulated, for example, allowing different therapies to be applied to the subject. Thus by selectively stimulating microstructures, to thereby selectively release therapeutic materials, this could be used in order to provide dosage control, or to deliver different therapeutic materials.

The monitoring device could also be used to generate an output, such as an output indicative of the indicator or a recommendation based on the indicator and/or cause an action to be performed. Thus, the monitoring device could be configured to generate an output including a notification or an alert. This can be used to trigger an intervention, for example, indicating to a user that action is required. This could simply be an indication of an issue, such as telling a user they are dehydrated or have elevated troponin levels and/or could include a recommendation, such as telling the user to rehydrate, or seek medical attention or similar. The output could additionally and/or alternatively, include an indication of an indicator, such as a measured value, or information derived from an indicator. Thus, a hydration level or analyte level or concentration could be presented to the user.

The monitoring device could also be configured to trigger other actions. For example, the output could be used to alert a caregiver that an intervention is required, for example transferring a notification to a client device and/or computer of the caregiver. In another example, this could also be used to control remote equipment. For example, this could be used to trigger a drug delivery system, such as an electronically controlled syringe injection pump, allowing an intervention to be triggered automatically. In a further example, a semi-automated system could be used, for example providing a clinician with a notification including an indicator, and a recommended intervention, allowing the clinician to approve the intervention, which is then performed automatically.

In one example, the monitoring device is configured to interface with a separate processing system, such as a client device and/or computer system. In this example, this allows processing and analysis tasks to be distributed between the monitoring device and the client device and/or computer system. For example, the monitoring device could perform partial processing of measured response signals, such as filtering and/or digitising these, providing an indication of the processed signals to a remote process system for analysis. In one example, this is achieved by generating subject data including the processed response signals, and transferring this to a client device and/or computer system for analysis. Thus, this allows the monitoring device to communicate with a computer system that generates, analyses or stores subject data derived from the measurement data. This can then be used to generate an indicator at least partially indicative of a health status associated with the subject.

It will also be appreciated that this allows additional functionality to be implemented, including transferring notifications to clinicians, or other caregivers, and also allowing for remote storage of data and/or indicators. In one example, this allows recorded measurements and other information, such as derived indicators, details of applied stimulation or therapy and/or details of other resulting actions, to be directly incorporated into an electronic record, such as an electronic medical record.

In one example, this allows the system to provide the data that will underpin the growing telehealth sector empowering telehealth systems with high fidelity and accurate clinical data to enable remote clinicians to gain the information they require, and they will be highly valued both in central hospitals and in rural areas away from centralized laboratories and regional hospitals. With time to treatment a strong predictor of improved clinical outcomes with heart attack patients, decentralized populations cannot rely solely on access to conventional large-scale hospitals. Accordingly, the system can provide a low cost, robust and accurate monitoring system, capable for example of diagnosing a heart attack, and yet being provided at any local health facility and as simple as applying a patch device. In this example, resources could be dispatched quickly for patients who test positive to troponin I, with no delay for cardiac troponin laboratory blood-tests. Similarly patients determined to be low-risk could be released earlier and with fewer invasive tests, or funnelled into other streams via their GP etc.

In a further example, a client device such as a smart phone, tablet, or the like, is used to receive measurement data from the wearable monitoring device, generate subject data and then transfer this to the processing system, with the processing system returning an indicator, which can then be displayed on the client device and/or monitoring device, depending on the preferred implementation.

However, this is not essential and it will be appreciated that some or all of the steps of analysing measurements, generating an indicator and/or displaying a representation of the indicator could be performed on board the monitoring device.

Again, it will be appreciated that similar outputs could also be provided to or by a remote processing system or client device, for example, alerting a clinician or trainer that a subject or athlete requires attention, that an intervention should be performed, controlling equipment, such as drug delivery devices, or the like.

The reader could be configured to perform measurements automatically when integrated into or permanently/semi permanently attached to the patch, or could perform measurements when brought into contact with the patch if the reader is separate. In this latter example, the reader can be inductively coupled to the patch.

Thus, it will be appreciated that functionality, such as processing measured response signals, analysing results, generating outputs, controlling measurement procedures and/or therapy delivery could be performed by an on-board monitoring device, and/or could be performed by remote computer systems, and that the particular distribution of tasks and resulting functionality can vary depending on the preferred implementation.

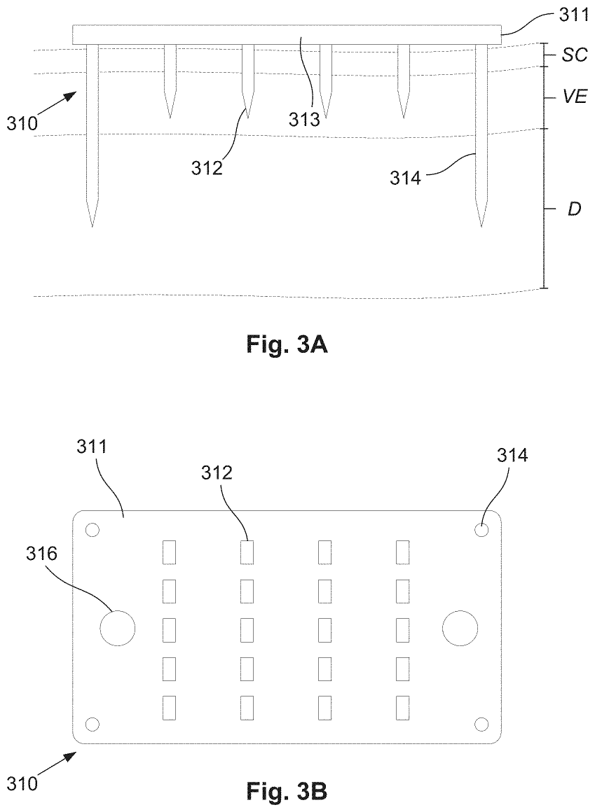

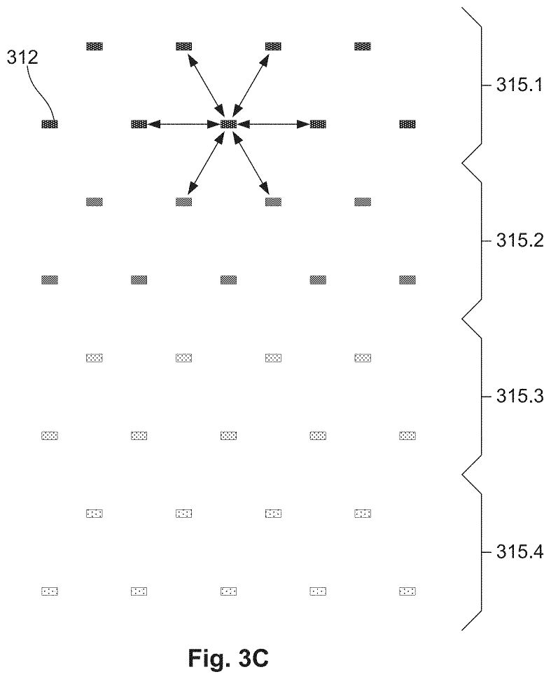

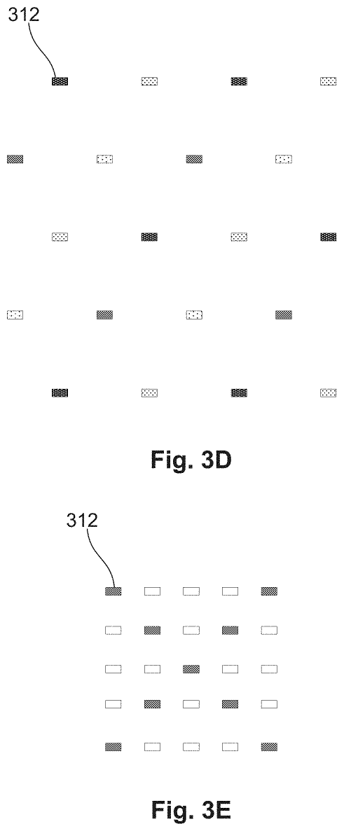

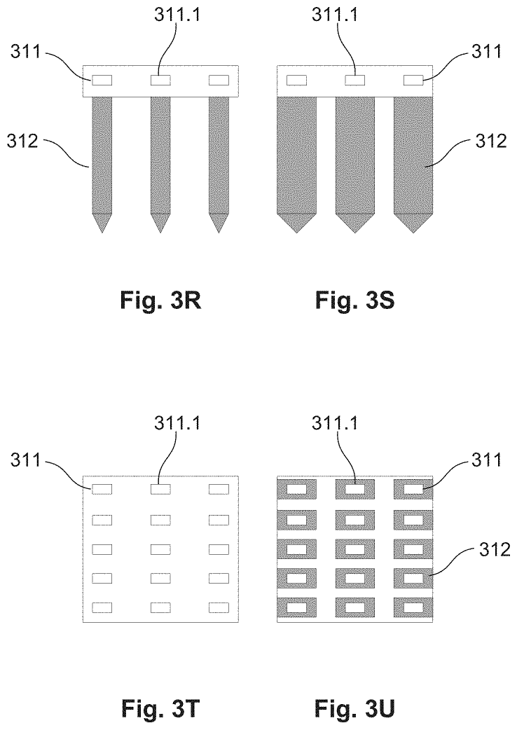

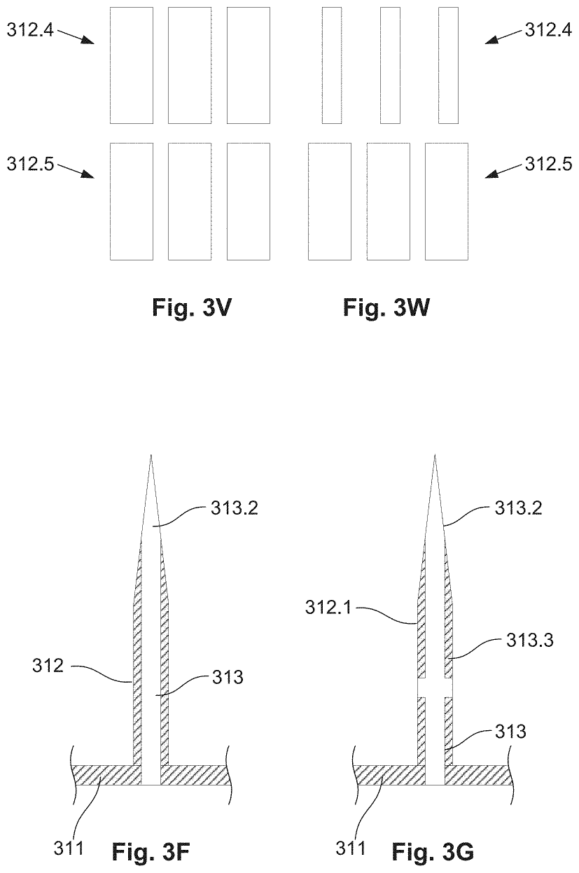

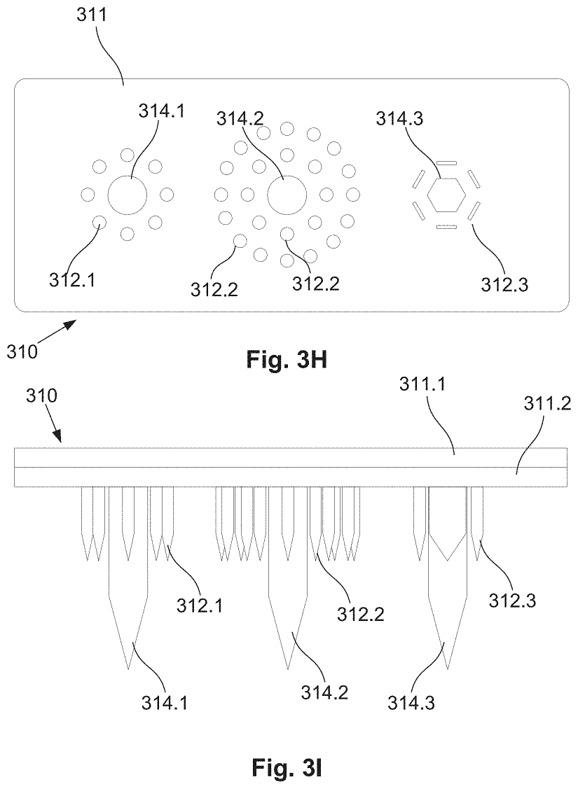

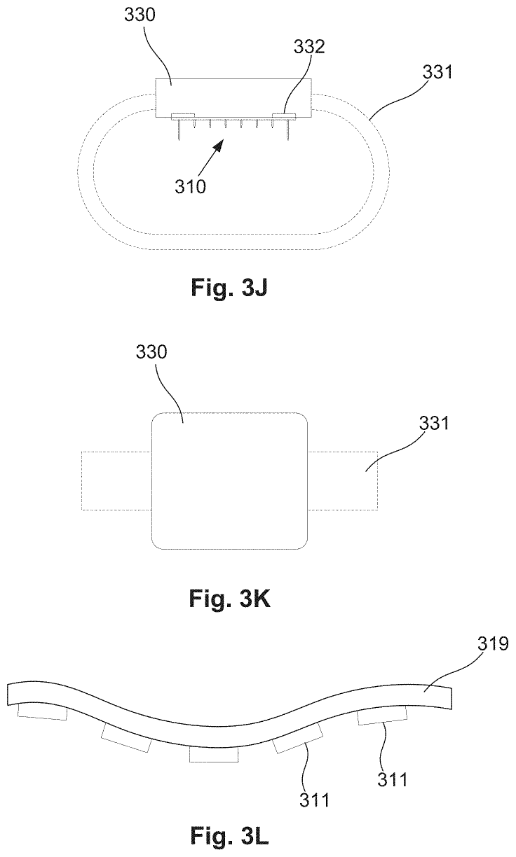

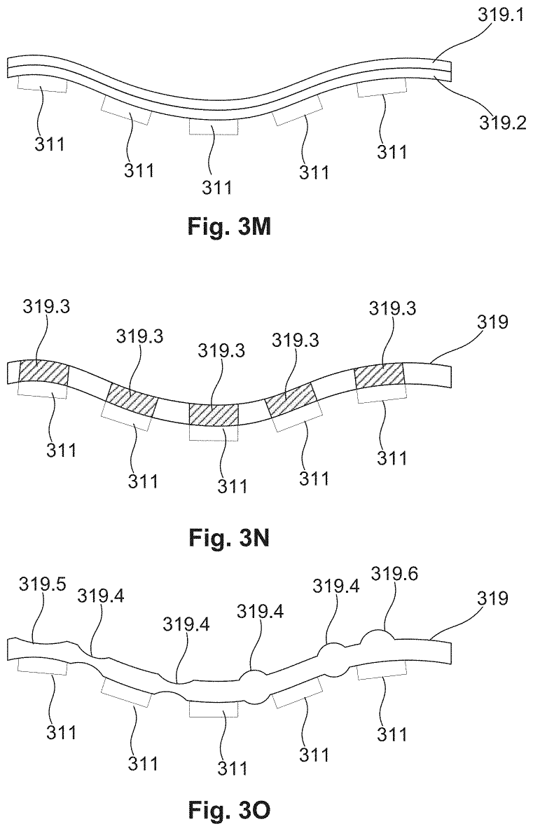

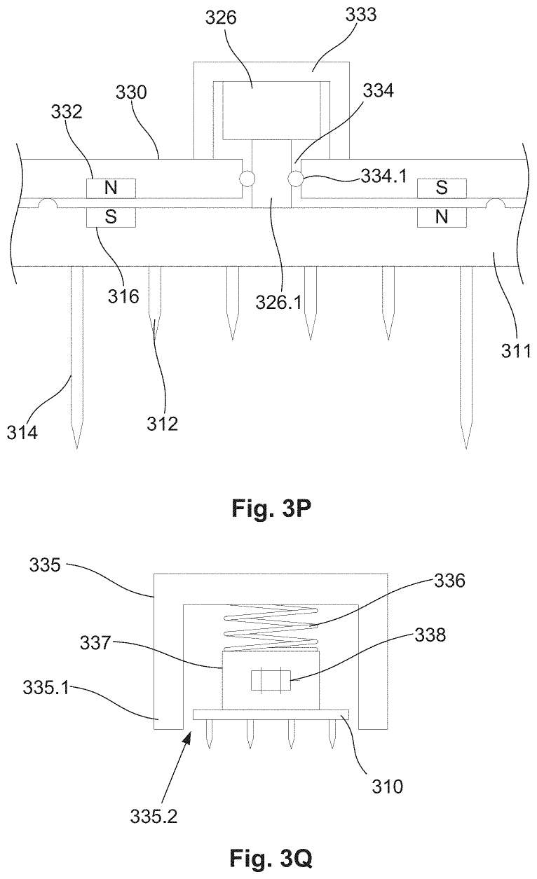

A further example of a system for performing measurements in the biological subject will now be described with reference to FIGS. 3A to 3P.

In this example, the system includes a patch 310, including a substrate 311 and microstructures 312. In this example, the patch further includes anchor microstructures 314 that are configured to penetrate into the dermis and thereby assist in securing the patch to the subject.

An example of the patch 310 is shown in more detail in FIG. 3B. In particular, in this example the substrate 311 is generally rectangular, with round corners to avoid discomfort when the substrate is applied to the subject's skin. The substrate 311 includes anchor microstructures 314 are provided proximate corners of the substrate 311 to help secure the substrate, whilst measurement microstructures 312 are arranged in an array on the substrate. In this example, the array has a regular grid formation, with the microstructures 312 being in provided in equally spaced rows and columns, but this is not essential and alternative spacing configurations could be used.

For example, the microstructures can be provided in a hexagonal grid arrangement as shown in FIG. 3C. This is particularly advantageous as each microstructure is equally spaced to all of the nearest neighbour microstructures, providing an even distribution of microstructures.

As shown in this example, the microstructures 312 are arranged in four groups 315.1, 315.2, 315.3, 315.4, with each group of microstructures 312 being configured to respond to different analytes and/or different concentrations of analytes, allowing a presence, absence, level or concentration of different analytes to be detected. Although the groups are spatially distinct in this example, it will be appreciated that this is not essential and alternatively the microstructures of different groups could be spatially interspersed, as shown in FIG. 3D.

Similarly, different microstructures could be provided in order to define patterns. An example pattern is shown in FIG. 3E, but it will be appreciated that the pattern could be of any form and could include alphanumeric characters, icons, or the like. The patterns could be associated with visual markings on the substrate, allowing the pattern to be interpreted. For example, the microstructures could be arranged to define indicators that are displayed relative to a scale, to show a concentration of analytes.

A further example arrangement is shown in FIGS. 3R to 3U.

In this example, microstructures 312 extend downwardly from the substrate 311 and are aligned with masking elements 311.1 in the substrate 311. The microstructures 312 are responsive to conditions in the subject, for example undergoing swelling when exposed to fluids. This could be achieved by having the microstructures 312 include or be coated with a hydrogel or other suitable material. Additionally, the microstructures are coloured, allowing these to be easily viewed.

In this instance, when fluid levels are low, the microstructures are hidden by the masking elements 311.1 and are not visible as shown in FIG. 3T. In contrast, upon exposure to ISF or other fluids, the microstructures swell and hence become visible from above as shown in FIG. 3U.

It will therefore be appreciated that as a whole the substrate will have a different appearance, for example undergoing a colour change, allowing changes in fluid levels to be easily monitored.

A further example arrangement is shown in FIGS. 3V and 3W.

In this example, first and second rows 312.4, 312.5 of plate microstructures are provided. Microstructures in the first row 312.4 include a hydrogel, either within the microstructure body or as a coating, which swells in the presence of fluid, whilst the microstructures in the second row are control microstructures that do not change shape. In this instance, when exposed to ISF or other fluids, microstructures in the first row 312.4 have a similar size to microstructures in the second row 512.5, as shown in FIG. 3V. In contrast, if ISF levels reduce, for example if the subject is dehydrated, the microstructures in the first row 312.4 shrink as shown in FIG. 3W, resulting in a different appearance.

In this regard, it will be appreciated that including the reference microstructures in the second row, allows the shape changes to be more easily identified. Additionally, to make visual perception of the change even more apparent, the microstructures can be positioned so as to establish patterns, such as a Moire effect. The Moire effect occurs when viewing a set of lines or dots that is superimposed and/or in close proximity to another set of lines or dots, where the sets differ in relative size, angle, or spacing. Thus, suitable configuration of the microstructures allows a shape change in some of the microstructures to be more easily identified, thus allowing a user to readily identify changes in measurements.

In other approaches, passive sensing could be used using self-assembled photonic crystals, formed in a hydrogel through a colloidal solution that is polymerised. In this instance, swelling leads to a change in periodicity and a corresponding change in the photonic crystal passband, which can be observed. Thus, forming microstructures from self-assembled photonic crystals in a hydrogel, again allows visual observations of hydration levels to be observed.

A specific example of a microstructure is shown in FIG. 3F and FIG. 3G, which includes a body 312 containing a core 313 that is substantially transparent to electromagnetic radiation at wavelengths of interest, such as a polymer or other similar material, covered by an opaque layer 313.1, which in one example could be a different polymer or other material configured to provide additional physical support and thereby strengthen the microstructure. In this instance, the core 313 terminates at an outlet 313.2 allowing electromagnetic radiation to be emitted from or received via the outlet. Ports 313.3 may also be provided extending through the opaque layer, at different locations along the microstructure, allowing radiation to be received from midway along the structure as shown at FIG. 3G. It will be appreciated that this arrangement allows radiation to be transmitted into and received from the subject, allowing the appearance of the patch to be influenced, as will be described in more detail below.

A further example microstructure arrangement is shown in FIGS. 3H and 3I. In this example, three anchor microstructures 314.1, 341.2, 314.3 are provided, surrounded by respective circumferentially spaced microstructures 312.1, 312.2, 312.3. This can be useful to maximise the effectiveness of the anchor, specifically providing the microstructures 312 in close proximity to the anchor microstructures 314 to avoid movement of the microstructures 312 within the subject. Additionally, in this example, the anchor microstructures 314 could be used in measuring or applying signals, for example by acting as a ground connection, or similar.

In this example, the substrate is also formed from multiple substrate layers 311.1, 311.2, which can assist in creating internal structures. In a manner similar to that described below with respect to a backing, the substrate could also include different regions or layers having different material properties, or the like.

In this example, the anchor microstructure 314.1 is circular and includes a single surrounding group of circumferentially spaced microstructures 312.1. However, it will be appreciated that this is not essential, and in the case of the anchor microstructure 314.2, the anchor microstructure 314.2 is surrounded by two or more concentric groups of microstructure 312.2, with the outer group including a larger number of microstructures. This allows a greater range of detection to be performed. It will be appreciated that other arrangements are also possible, such as providing further concentric groups, different numbers of microstructures in each group, or the like. Additionally, whilst circular groups are shown, this is not intended to be limiting, and other shapes or distributions could be used including oval shaped, square shaped, or similar.

In the case of the anchor microstructure 314.3 this is hexagonal, with six plate microstructures 312.3, each being positioned radially outwardly from a respective face of the hexagonal anchor microstructure 314.3, which can be useful to maximise a surface area of electrodes on each face and plate, whilst maintaining equidistant separation between the anchor and surrounding microstructures.

Whilst the above configurations have been described with respect to anchor microstructures, this is not essential and it will be appreciated that similar arrangements could be used with any microstructure. For example, a single microstructure could be used to provide stimulation, such as by releasing a material into the subject, with multiple surrounding detection microstructures being used to detect analytes, or a single detection microstructure could be used with multiple surrounding stimulation microstructures. This provides an effective master slave arrangement, in which a single master stimulation/detection microstructure is used with multiple detection/stimulation microstructures.

Such master/slave relationships can be used in wide range of applications, for example to use a single stimulation to induce responses in multiple detection microstructures, which could be used to detect different analytes, for example using different coatings or similar, so that a single stimulation can trigger detection of different analytes.

The substrate can also include coupling members 316, such as magnets, which can be used to attach the substrate to a housing.

In the example of FIGS. 3J and 3K, the housing 330 is a generally rectangular housing. The measuring device can optionally have a form factor similar to a watch, or other wearable device, in which case a strap 331 is included that allows the housing to be secured to the user. However, this is not essential and other securing mechanisms could be used. Alternatively, the housing could simply be brought into engagement with the patch and held in position each time a measurement is performed. In this example, the housing includes coupling members 332, such as magnets, or the like, which can engage with corresponding coupling members 316 on the substrate allowing the substrate to be secured to the housing. Whilst any form of coupling member could be used, the use of magnets is particularly advantageous as these can be contained within the housing 330, allowing the housing to be sealed, and can also act to ensure correct alignment of the substrate 310, for example by having polarities of the magnets guide a relative orientation of the substrate 310 and housing 330.

However, it will be appreciated that this configuration is for the purpose of illustration only, and other arrangements could be used. For example, the substrate could form part of an adhesive patch, which is applied to the subject and retained in place. Alternatively, adhesive could be provided on a surface of the substrate to adhere the substrate directly to the subject. The housing 330, could then be selectively attached to the patch, for example, using magnetic coupling, thereby allowing measurements to be performed as needed.

In this example, the substrate could be a flexible substrate, which can be achieved using a woven or non-woven fabric or other suitable material, with microstructures directly attached thereto. More typically however, flexibility is achieved using a number of individual substrates 311 mounted on a flexible backing 319, to form a segmented substrate, as shown in FIG. 3L. It will be appreciated that such arrangements can be used in a wide variety of circumstances, including having the substrates mounted to a strap or the like, for attachment to the subject.

A number of further variations are shown in FIGS. 3M to 3O.

Specifically in the example of FIG. 3M, the backing 319 is formed from multiple backing layers 319.1, 319.2, with two being shown in the example for the purpose of illustration only. The use of multiple layers can be beneficial in achieving desired properties, for example to provide adhesive, or waterproof layers, or the like.

In the example of FIG. 3N, the backing layer has multiple interspersed regions 319.3, which can be used for particular purposes, such as to allow for easier attachment of the substrates 311, to provide connectivity to a measuring device 320, to allow for increased flexibility between the substrates 311, or the like. In this example, interspersed regions are substantially aligned with the substrates, although it will be appreciated that this is not essential, and they could be provided at other locations.

A further example is shown in FIG. 3O, which includes a number of shape modifications, including thinner regions 319.4, located between substrates, which could be used to enhance flexibility, or thicker regions 319.5 between the substrates, which could increase strength. Similarly thinner or thicker regions 319.5, 319.6 could be provided in line with the substrates, for example to enhance strength, flexibility, connection to a measuring device, or the like.

Whilst these features have been described with reference to a backing layer, it will be appreciated that similar approaches could be used for the substrate itself.

An example of an actuator configuration to assist with applying a patch will now be described with reference to FIG. 3P.

In this example, the housing 330 includes a mounting 333 to which the actuator 326, such as a piezoelectric actuator, or vibrating motor, is attached. The actuator 326 is typically a spring or electromagnetic actuator in combination with a piezoelectric actuator or vibratory motor coupled to the housing, to bias and vibrate the substrate relative to an underside of the housing, to thereby urge the microstructures into the skin, whilst the transceiver is typically a short-range wireless transceiver, such as a Bluetooth system on a chip (SoC).

The patch substrate 311 is positioned adjacent the underside of the housing 330, with magnets 316, 332 being arranged to urge the substrate 311 towards the housing 330. The arm 326.1 engages the substrate to thereby transmit forces from the actuator 326 to the substrate 311, allowing the substrate and hence microstructures 312, 314, to be vibrated to aid insertion of the microstructures into the subject. Specifically, this arrangement transmits forces directly to the substrate 311, allowing forces in the substrate to be maximised, whilst minimising vibration of the housing 330.

A further example actuator arrangement will now be described with reference to FIG. 3Q.

In this example, the actuator arrangement includes an actuator housing 335 having a base 335.1 including an opening 335.2. The housing contains a spring 336 and mounting 337, which in use supports a patch 310 (and optional integrated reader). The mounting also optionally contains a piezoelectric actuator or offset motor 338.

In use, the actuator housing 335 is positioned so that a base 335.1 of the housing 335 abuts against the subject's skin, with the patch at least partially projecting through the opening 335.2. In one example, this is achieved by having an operator hold the actuator housing. However, this is not essential and additionally and/or alternatively, the actuator housing could be integrated into and/or form part of a monitoring device as described above.

In use, the spring 336 is configured to apply a continuous biasing force to the mounting 337, so the patch 310 is urged against the subject's skin. Additionally, the piezoelectric actuator or offset motor 338 can cause the mounting 337, and hence patch 310, to vibrate, thereby facilitating piercing and/or penetration of the stratum corneum by the microstructures.

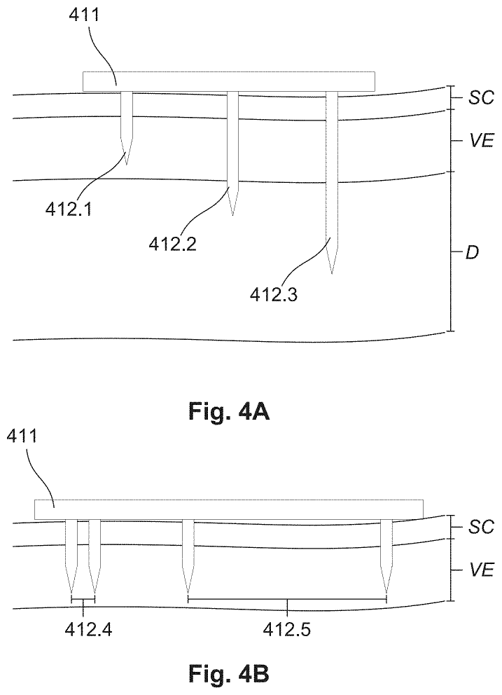

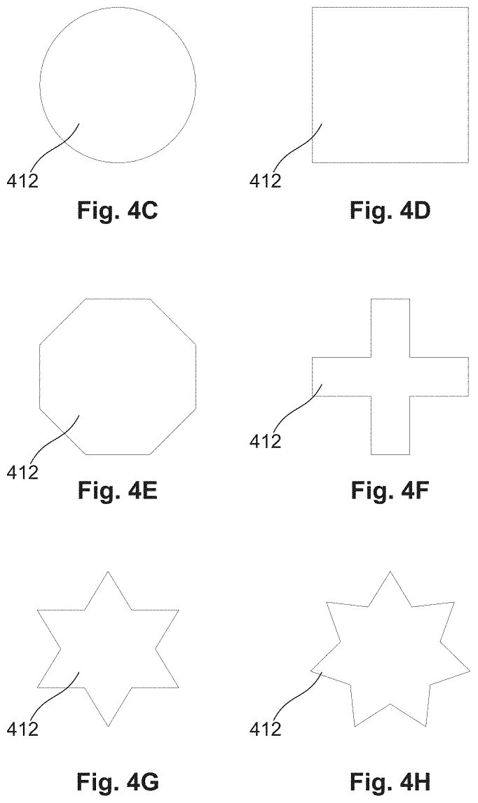

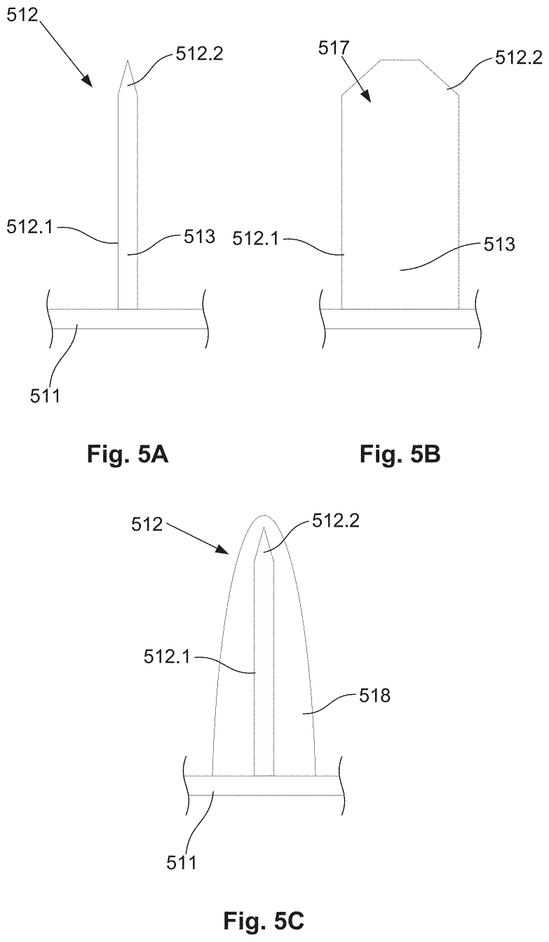

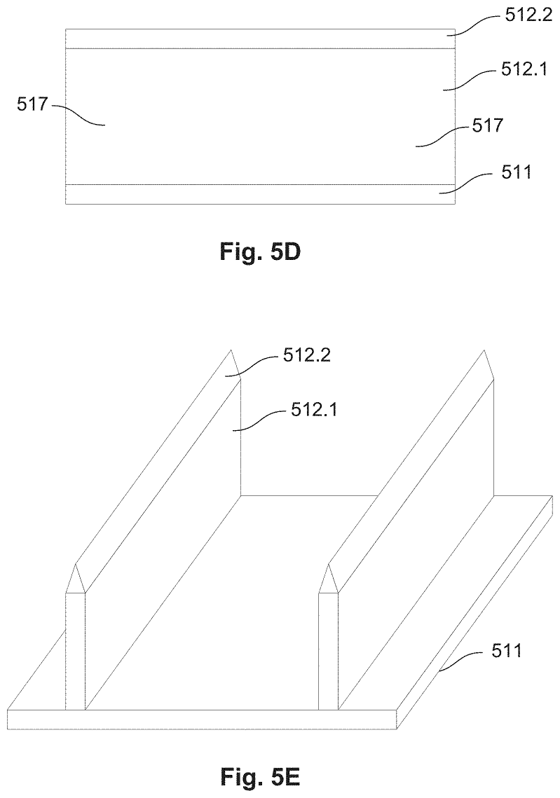

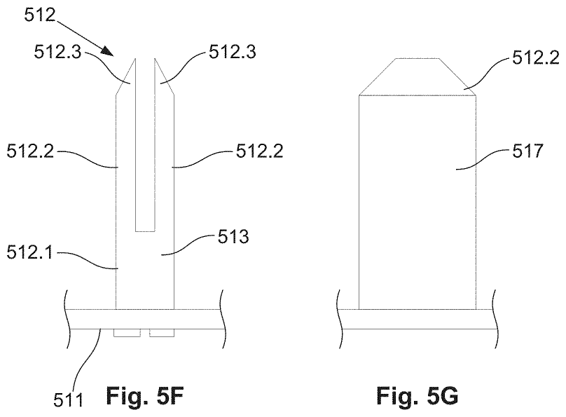

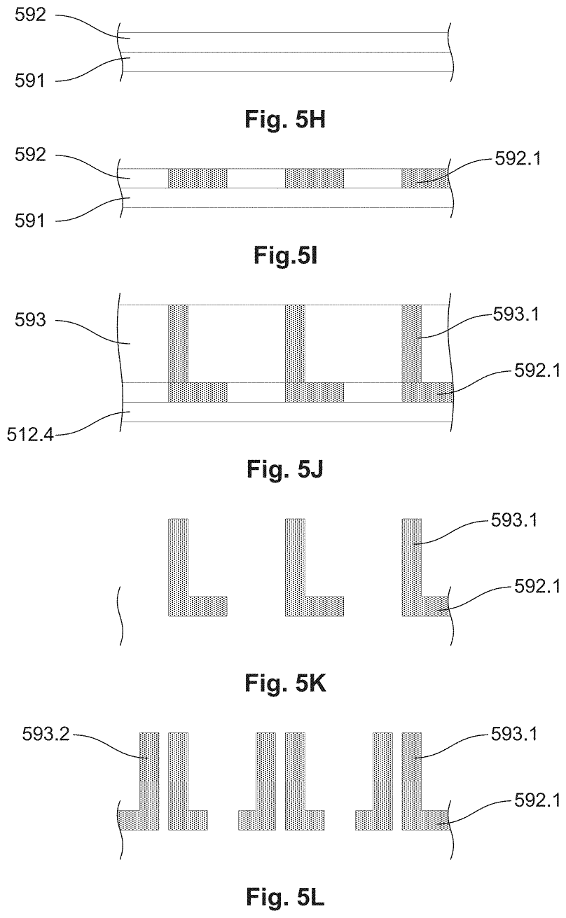

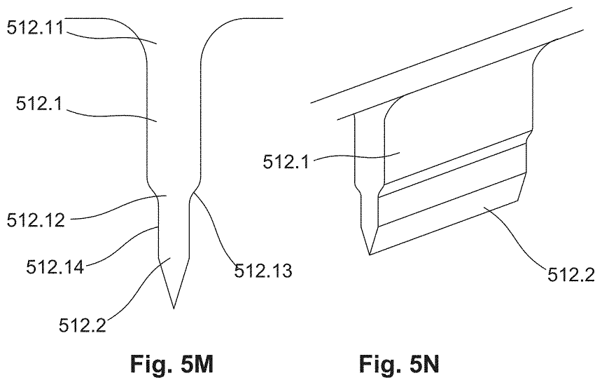

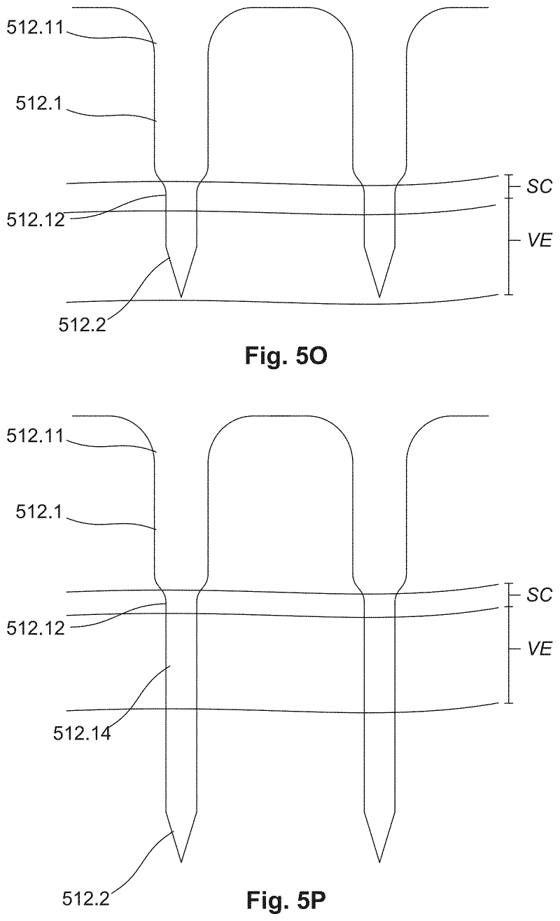

Example microstructure arrangements will now be described in more detail with reference to FIGS. 4 and 5.

In the example of FIG. 4A, different length microstructures are shown with a first microstructure 412.1 penetrating the stratum corneum and viable epidermis, but not breaching the dermis, a second microstructure 412.2 entering the dermis but only just passes the dermal boundary, whereas a third microstructure 412.3 penetrates the dermal layer at greater distance. It will be appreciated that the length of structure used will vary depending upon the intended application of the device, and specifically the nature of the barrier to be breached.

In the example of FIG. 4B, pairs of microstructures are provided with a first microstructure pair 412.4 having a closer spacing and a second microstructure pair 412.5 having a relatively large spacing.

The microstructures can have a range of different shapes, and examples are shown in FIGS. 4C to 4H. Specifically, these illustrate circular, rectangular, octagonal, cruciform, and star shapes. The shapes used will vary depending on the intended application. For example, larger numbers of the microstructures can be useful to provide multiple different electrode surfaces, whilst a greater overall surface area can be useful to maximise the amount of coating. Similarly, acute angled surfaces can, such as the cruciform and star arrangements, can allow coating to be used to provide an overall circular profile, with different coating depths around the microstructure.

A specific example of a plate microstructure is shown is shown in FIGS. 5A to 5C.

In this example, the microstructure is a plate having a body 512.1 and a tip 512.2, which is tapered to facilitate penetration of the microstructure 512 into the stratum corneum. This arrangement can provide a large surface area, which can be useful for observing visual changes, or can be used to allow a coating 518 to be applied and maximise the coating amount whilst still allowing easy penetration of the subject.

A further example of a blade microstructure is shown is shown in FIGS. 5D and 5E.

In this example, the microstructure is an elongate body 512.1 and tip 512.2, which is tapered to facilitate penetration of the microstructure 512. This is generally similar in profile to the plate arrangement described above, but in this example is significantly wider, and in one particular example, can extend substantially the entire distance across the substrate.

In the example, shown the blade tip is parallel to the substrate, but this is not essential and other configurations could be used, such as having a sloped tip, so that the blade penetrates progressively along the length of the blade as it is inserted, which can in turn facilitate penetration. The tip may also include serrations, or similar, to further enhance penetration.

A further example arrangement is shown at FIGS. 5F and 5G, with the microstructure again including a generally similar plate like arrangement, with the microstructure including spaced apart prongs 612.2, which can be used for observing changes in tissue or other substances between the prongs 612.2.

A technique for manufacturing microstructures will now be described with reference to FIGS. 5H to 5L.

In this example, a carrier wafer 591 is provided and spin coated with a photopolymer layer 592. The photopolymer layer 592 is selectively exposed to UV illumination and crosslinked, to create structural regions 592.1, which in this example form a substrate. A second photopolymer layer 593 is spun coated onto the first layer 591, and exposed to UV illumination and cross linked to form second structural regions 593.1, which in this example form microstructures, extending from the substrate. The carrier wafer and non-crosslinked polymer are removed to create the microstructures shown in FIG. 5K.

It will be appreciated that this layering technique can be used to create a wide range of different microstructure configurations, and alternative design is shown in FIG. 5L.

Specific examples of microstructures for performing measurements in the epidermis are shown in FIGS. 5M to 5P.

In this example, the microstructures are plates or blades, having a body 512.1, with a flared base 512.11, where the body joins the substrate, to enhance the strength of the microstructure. The body narrows at a waist 512.12 to define shoulders 512.13 and then extends to a tapered tip 512.2, in this example, via an untapered shaft 512.14. Typical dimensions are shown in Table 2 below.

TABLE 2

| Parameter | Min. | Typical | Max. | Units |
|---|---|---|---|---|
| Length | 50 | 150 | 300 | microns |
| Width | 50 | 150 | 300 | microns |
| Thickness | 10 | 25 | 50 | microns |
| Density | 100 | 600 | 5000 | cm⁻² |
| Tip radius | 0.1 | 1 | 5 | microns |
| Surface area per | 2,000 | 22,500 | 200,000 | micron² |
| electrode | | | | |
| Buttress width at | 30 | 75 | 150 | microns |
| base | | | | |

An example of a pair of the microstructures of FIGS. 5M and 5N on insertion into a subject is shown in FIG. 5O.

In this example, the microstructures are configured so that the tip 512.2 penetrates the stratum corneum SC and enters the viable epidermis VE. The waist 512.12, and in particular the shoulders 512.13 abut the stratum corneum SC so that the microstructure does not penetrate further into the subject, and so that the tip is prevented from entering the dermis. This helps avoid contact with nerves, which can lead to pain.

In this configuration, the shaft 512.14 and tip 512.2 can be coated so that detection is performed in the viable epidermis VE.

However, it will be appreciated that other configurations can be used. For example, in the arrangement of FIG. 5P, the shaft 512.14 is lengthened so the tip 512.2 enters the dermis, allowing dermal (and optional epidermal) measurements to be performed.

In this example, typical dimensions are shown in Table 3 below.

TABLE 3

| Parameter | Min. | Typical | Max. | Units |
|---|---|---|---|---|
| Length | 50 | 250 | 450 | microns |
| Width | 50 | 250 | 450 | microns |
| Thickness | 10 | 30 | 50 | microns |
| Density | 100 | 600 | 5000 | cm⁻² |
| Tip radius | 0.1 | 1 | 5 | microns |
| Surface area per | 10,000 | 62,500 | 427,000 | micron² |
| electrode | | | | |
| Buttress width at | 30 | 75 | 150 | microns |
| base | | | | |

An example of the spacing for these configurations are shown in Table 4 below.

TABLE 4

| Parameter | Min. | Typical | Max. | Units |
|---|---|---|---|---|
| Separation | 200 | 500 | 1000 | microns |
| between | | | | |
| microstructures | | | | |

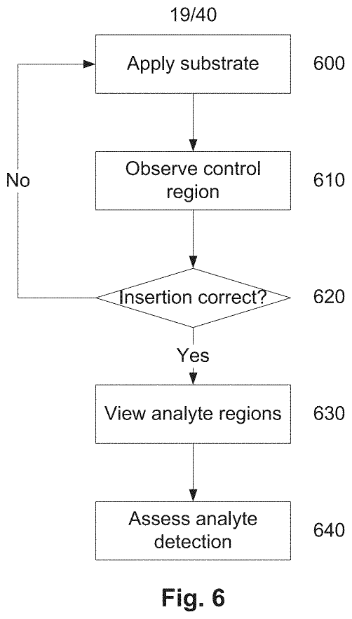

An example of a process for using a system described above will now be described with reference to FIG. 6.

In this example, at step 600, the substrate is initially applied to the subject, typically using the actuator arrangement, with a control region being observed at step 610. This is performed to allow a visual indication to be used to confirm the patch has been successfully applied. If it is determined insertion is not correct at step 620, the process can be repeated.

Otherwise, at step 630, one or more analyte detection regions are observed, allowing this to be used to assess analyte detection at step 640, for example using this to determine a presence, absence, level or concentration of one or more analytes in the subject.

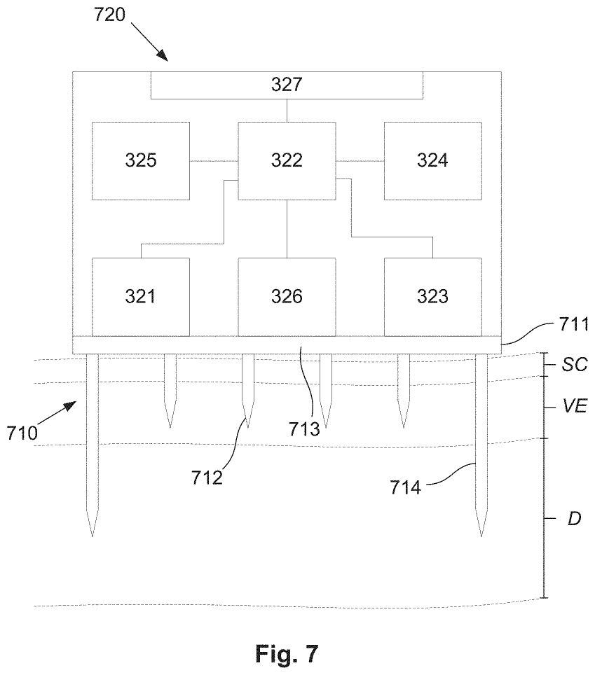

This process is described as a purely manual process, with no additional equipment being required other than the patch. However, this is not essential, and as previously mentioned the system can be used in conjunction with an illumination source and sensor. In one example, these are incorporated into a measuring device, an example of which will now be described with reference to FIG. 7.

In this example, the monitoring device 720, including a sensor 321 and one or more electronic processing devices 322. The system further includes a signal generator 723, a memory 724, an external interface 725, such as a wireless transceiver, an actuator 726, and an input/output device 727, such as a touchscreen or display and input buttons, connected to the electronic processing device 722. The components are typically provided in housing 330, as previously described. For ease of illustration the remaining description will refer generally to a processing device, but it will be appreciated that multiple processing devices could be used, with processing distributed between the devices as needed, and that reference to the singular encompasses the plural arrangement and vice versa.

The nature of the signal generator 723 and sensor 721 will vary, but typically include a laser, LED, or other electromagnetic radiation source and photodiode or CCD sensor, or the like. The actuator 726 is typically a piezoelectric actuator coupled to the housing, to vibrate an underside of the housing, whilst the transceiver is typically a short-range wireless transceiver, such as a Bluetooth system on a chip (SoC).

The processing device 722 executes software instructions stored in the memory 724 to allow various processes to be performed, including controlling the signal generator 723, receiving and interpreting signals from the sensor 721, generating measurement data and transmitting this to a client device or other processing system via the transceiver 725. Accordingly, the electronic processing device is typically a microprocessor, microcontroller, microchip processor, logic gate configuration, firmware optionally associated with implementing logic such as an FPGA (Field Programmable Gate Array), or any other electronic device, system or arrangement.

In use the monitoring device 720 is coupled to a patch 710, including a substrate 711 and microstructures 712, which are exposed to radiation from the signal generator 723, and with the sensor 721 receiving radiation reflected from or emitted from the patch.

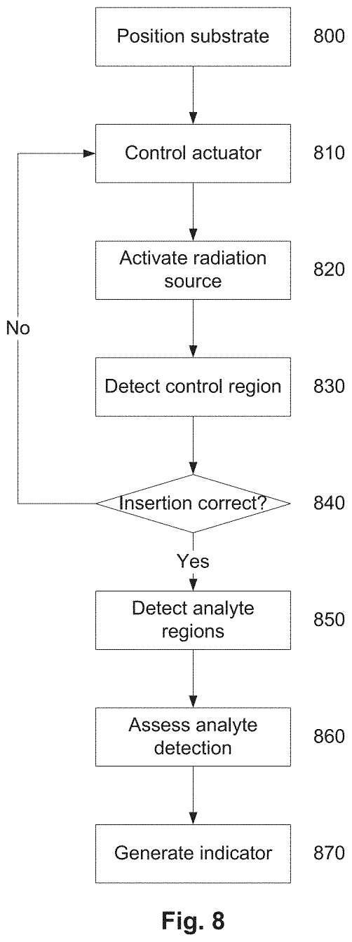

An example of a process for using the above described measuring device will now be described with reference to FIG. 8.

In this example, at step 800, the substrate is initially positioned on subject, with the actuator 726 being used at step 810 to insert the patch, and in particular the microstructures, into the subject. The radiation source 723 is activated at step 820, allowing a control region of the patch to be detected by the sensor 721 at step 830. This is performed to allow the processing device 722 to use a change in appearance of the control region to confirm the patch has been successfully applied, with this being used to control the actuator until correct insertion is achieved at step 840.

Following this, at step 850, the radiation source 723 and sensor 721 are used to detect electromagnetic properties of different analyte regions on the patch. In this regard, it will be appreciated that the processing device 722 may be programmed with details of different regions, specifically identifying the analyte or concentration that is detected by each region, and what the electromagnetic properties of each region represent. For example, this might indicate that if the light emitted or reflected by a region exceeds a threshold, this indicates a certain concentration of analytes. Accordingly, analysing the electromagnetic response of each region allows the processing device 722 to assess analyte detection at step 860, for example using this to determine a presence, absence, level or concentration of one or more analytes in the subject.

At step 870 an indicator can be generated, and optionally stored or displayed on the display 727, allowing a user to view information regarding the presence, absence, level or concentration of analytes.

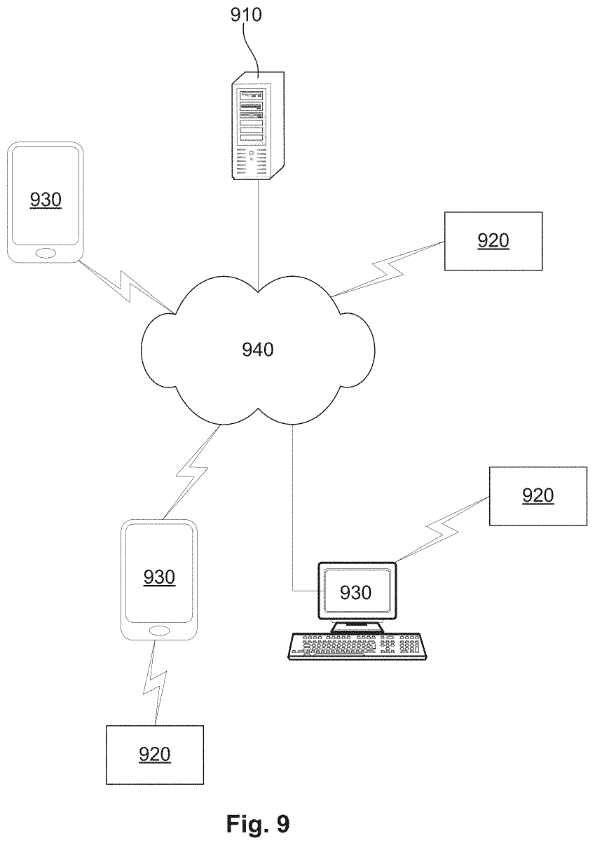

In one example, the monitoring device operates as part of a distributed architecture, an example of which will now be described with reference to FIG. 9.

In this example, one or more processing systems 910 are coupled via communications networks 940, and/or one or more local area networks (LANs), to a number of client devices 930 and monitoring devices 920. The monitoring devices 920 could connect direction to the networks, or could be configured to connect to a client device 930, which then provides onward connectivity to the networks 940. It will be appreciated that the configuration of the networks 940 are for the purpose of example only, and in practice the processing systems 910, client devices 930 and monitoring devices 930 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

In one example, each processing system 910 is configured to receive subject data from a monitoring device 920 or client device 930, and analyse the subject data to generate one or more health status indicators, which can then be provided to a client device 930 or monitoring device 920 for display. Whilst the processing system 910 is a shown as a single entity, it will be appreciated that the processing system 910 can be distributed over a number of geographically separate locations, for example by using processing systems 910 and/or databases that are provided as part of a cloud based environment. However, the above described arrangement is not essential and other suitable configurations could be used.

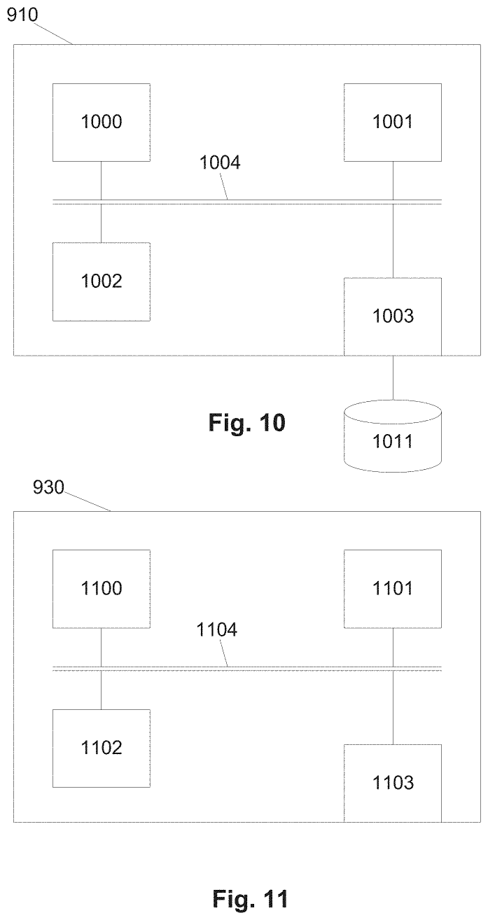

An example of a suitable processing system 910 is shown in FIG. 10.

In this example, the processing system 910 includes at least one microprocessor 1000, a memory 1001, an optional input/output device 1002, such as a keyboard and/or display, and an external interface 1003, interconnected via a bus 1004 as shown. In this example the external interface 1003 can be utilised for connecting the processing system 910 to peripheral devices, such as the communications network 940, databases 1011, other storage devices, or the like. Although a single external interface 1003 is shown, this is for the purpose of example only, and in practice multiple interfaces using various methods (eg. Ethernet, serial, USB, wireless or the like) may be provided.

In use, the microprocessor 1000 executes instructions in the form of applications software stored in the memory 1001 to allow the required processes to be performed. The applications software may include one or more software modules, and may be executed in a suitable execution environment, such as an operating system environment, or the like.

Accordingly, it will be appreciated that the processing system 910 may be formed from any suitable processing system, such as a suitably programmed client device, PC, web server, network server, or the like. In one particular example, the processing system 910 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

An example of a suitable client device 930 is shown in FIG. 11.

In one example, the client device 930 includes at least one microprocessor 1100, a memory 1101, an input/output device 1102, such as a keyboard and/or display, and an external interface 1103, interconnected via a bus 1104 as shown. In this example the external interface 1103 can be utilised for connecting the client device 930 to peripheral devices, such as the communications networks 940, databases, other storage devices, or the like. Although a single external interface 1103 is shown, this is for the purpose of example only, and in practice multiple interfaces using various methods (eg. Ethernet, serial, USB, wireless or the like) may be provided.

In use, the microprocessor 1100 executes instructions in the form of applications software stored in the memory 1101 to allow communication with the processing system 910 and/or monitoring device 920.

Accordingly, it will be appreciated that the client devices 1130 may be formed from any suitable processing system, such as a suitably programmed PC, Internet terminal, lap-top, or hand-held PC, and in one preferred example is either a tablet, or smart phone, or the like. Thus, in one example, the client device 1130 is a standard processing system such as an Intel Architecture based processing system, which executes software applications stored on non-volatile (e.g., hard disk) storage, although this is not essential. However, it will also be understood that the client devices 1130 can be any electronic processing device such as a microprocessor, microchip processor, logic gate configuration, firmware optionally associated with implementing logic such as an FPGA (Field Programmable Gate Array), or any other electronic device, system or arrangement.

Examples of the processes for performing measurements and generating indicators will now be described in further detail. For the purpose of these examples it is assumed that one or more processing systems 910 acts to analyse received subject data and generate resulting indicators. Measurements are performed by the monitoring devices 920, with subject data being transferred to the processing systems 910 via the client devices 230. In one example, to provide this in a platform agnostic manner, allowing this to be easily accessed using client devices 930 using different operating systems, and having different processing capabilities, input data and commands are received from the client devices 930 using via a webpage, with resulting visualisations being rendered locally by a browser application, or other similar application executed by the client device 930. The processing system 910 is therefore typically a server (and will hereinafter be referred to as a server) which communicates with the client device 930 and/or monitoring device 920, via a communications network 940, or the like, depending on the particular network infrastructure available.

To achieve this the server 910 typically executes applications software for hosting webpages, as well as performing other required tasks including storing, searching and processing of data, with actions performed by the processing system 910 being performed by the processor 1000 in accordance with instructions stored as applications software in the memory 1001 and/or input commands received from a user via the I/O device 1002, or commands received from the client device 1030.

It will also be assumed that the user interacts with the server 910 via a GUI (Graphical User Interface), or the like presented on the client device 930, and in one particular example via a browser application that displays webpages hosted by the server 910, or an App that displays data supplied by the server 910. Actions performed by the client device 930 are performed by the processor 1100 in accordance with instructions stored as applications software in the memory 1101 and/or input commands received from a user via the I/O device 1102.

However, it will be appreciated that the above described configuration assumed for the purpose of the following examples is not essential, and numerous other configurations may be used. It will also be appreciated that the partitioning of functionality between the monitoring devices 920, client devices 930, and the server 910 may vary, depending on the particular implementation.

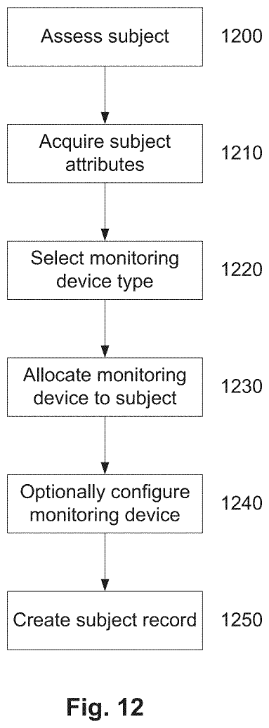

In one example, monitoring devices are allocated to respective users, with this allocation being used to track measurements for the subject. An example of a process for allocating a monitoring device 920 to a subject will now be described with reference to FIG. 12.

In this example, the subject initially undergoes an assessment at step 1200, with this process being performed by a clinician. The clinician will use the assessment to guide the type of monitoring that needs to be performed, for example to identify particular biomarkers that are to be measured, which in turn may depend on any symptoms or medical diseases, disorders or conditions suffered by the subject. As part of this process, the clinician will typically acquire subject attributes at step 1210, such as measurement of weight, height, age, sex, details of medical interventions, or the like. This can be performed using a combination or techniques, such as querying a medical record, asking questions, performing measurements or the like.

Once the assessment has been completed, a patch type can be selected at 1220, with this being performed based on the measurements that are required. In this regard, it will be appreciated that different combinations of microstructure arrangement and sensing modalities can be used in order to allow a range of different measurements to be performed, and it is therefore important that the correct selection is made to enable the measurements to be collected. A specific monitoring device 920 is then allocated to the subject at step 1230. In this regard, in each device will typically include a unique identifier, such as a MAC (Media Access Control) address or other identifier, which can be used to uniquely associate the monitoring device with the subject.

At step 1240 the monitoring device 920 can optionally be configured, for example to update firmware or the instruction set needed to perform the respective measurements. At step 1250, a subject record is created, which is used to store details associated with the subject, including subject attributes, subject data, indicators, or any other relevant information. Additionally, the subject record will also typically include an indication of the monitoring device identifier, thereby associating the monitoring device with the subject.

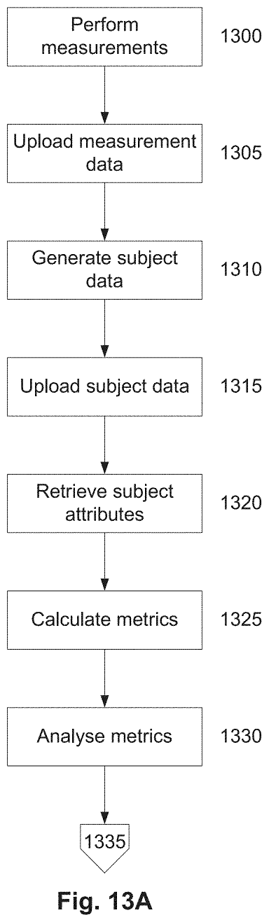

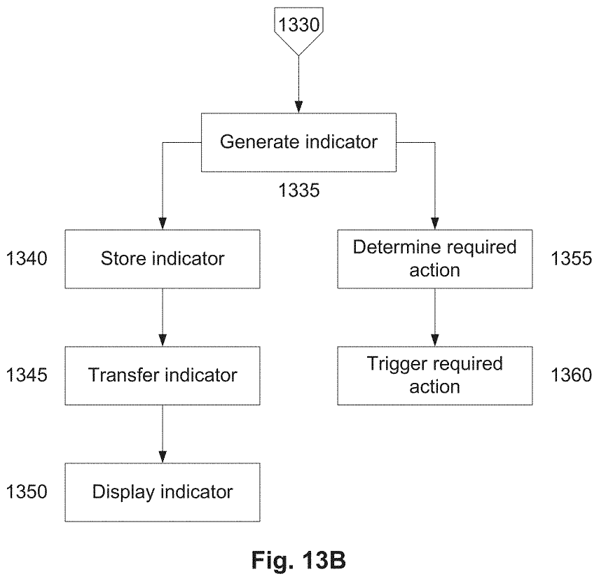

An example of the process of using the device to perform measurements will now be described with reference to FIGS. 13A and 13B.

In this example, at step 1300 one or more measurements are performed. The measurements are performed by utilising the process described above with respect to FIG. 8. Measurement data corresponding to measured electromagnetic responses of different regions of the patch is recorded with this being uploaded to the client device 930 at step 1305, allowing the client device 930 to generate subject data at step 1310. The subject data could simply be the measurement data, but may also include additional information provided by the client device 930. This allows user inputs to be provided via the client device 930, for example providing details of symptoms, changes in attributes or the like. The subject data is then uploaded to the server 910 at step 1315. The server 910 then retrieves one more subject attributes at step 1320, for example from the subject record, with the server 910 then calculating one or more metrics at step 1325.

At step 1330, the server 910 analyses the metrics. The manner in which this is performed will vary depending on the preferred implementation. For example, this could be achieved by applying the metrics to a computational model that embodies a relationship between a relevant health status and the one or more metrics. Alternatively, the metrics could be compared to defined thresholds, which can be established from a population of reference subjects, and which are used to represent certain diseases, disorders or conditions, such as the presence or absence of a medical condition. As a further option, the metrics could be compared to previous metrics for the subject, for example to examine changes in the metrics, which could in turn represent a change in health status. The results of the analysis can be used to generate one or more indicators at step 1335. In one example, the indicator can be in the form of a score representing a health status, or could be indicative of a presence, absence or degree of diseases, disorders or condition.

At step 1340 the indicator can be stored, with an indication of the indicator being transferred to the client device 930 at step 1345, allowing the indicator to be displayed, either by the client device 930 or the monitoring device 920 at step 1350.

Additionally, and/or alternatively, at step 1355 the indicator can be used to determine if an action is required, for example if an intervention should be performed. The assessment of whether an action is required could be performed in any one of a number of manners, but typically involves comparing the indicator to assessment criteria defining a predetermined threshold or range of acceptable indicator values. For example, comparing a hydration indicator to a range indicative of normal hydration, or comparing an analyte indicator indicative of a normal level or concentration of analytes.

The assessment criteria can also specify the action required if the indicator falls outside of the acceptable range, and any steps required to perform the action, allowing the action to be performed at step 1360. For example, in a theranostic application, this could involve causing the applying monitoring device to apply a stimulation signal to electrodes, thereby allowing one or more therapeutic agents to be released. Alternatively, if the subject is dehydrated, the action could include having the monitoring device provide a recommendation to the user to hydrate, whereas if certain analytes are detected, this could be indicative of a medical situation, in which the processing system or monitoring device could generate a notification which is provided to a clinician, or other nominated person or system, allowing them to be alerted. The notification could include any determined indicator and/or measured response signals, allowing the clinician to rapidly identify any interventions needed. Alternatively, the action could involve notifying the user, so for example, if the subject is dehydrated, the action could include having the monitoring device provide a recommendation to the user to hydrate.

It will be therefore be appreciated that this enables actions to be triggered as needed.

The above described processes describe transfer of data to remote systems for analysis, which can have a number of benefits. For example, this allows more complex analysis to be performed than would otherwise be the case with existing processing capabilities. This also allows remote oversight, for example, allowing a clinician to access records associated with multiple patients, in real-time, enabling the clinician to respond rapidly as needed. For example, in the event that measured data shows an indication of a deleterious health state, the clinician could be alerted or notified, allowing an intervention to be triggered. Additionally, collective monitoring provides public health benefits, for example to allow tracking of infectious diseases or similar. Furthermore, central analysis allows data mining to be used in order refine analysis processes, making this more accurate as more data is collected.

However, it will be appreciated that the distributed implementation is not essential, and additionally or alternatively, analysis could be performed in situ, with resulting information being displayed locally, for example, using a client device or in-built display.

It will be appreciated that the above described technique employs a patch with no electronically active sensing elements, and which in one example can be used without any measuring equipment, allowing measurements to be made regarding conditions within the subject, such as the presence or concentration of analytes, solely based on visual inspection of a patch. It will also be appreciated that suitably adapting the coating allows a range of different analytes to be sensed and that this can also be adapted for performing other suitable measurements.

Further details exemplifying the above described arrangements will now be described.

### Manufacture

Example process for manufacturing a substrate including microstructures will now be described in more detail.

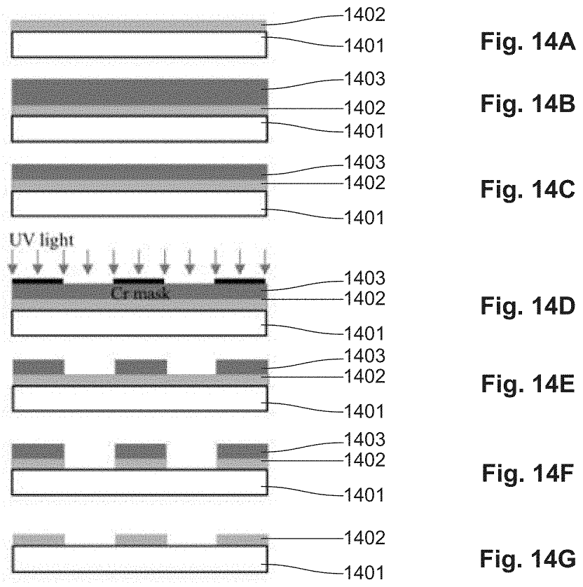

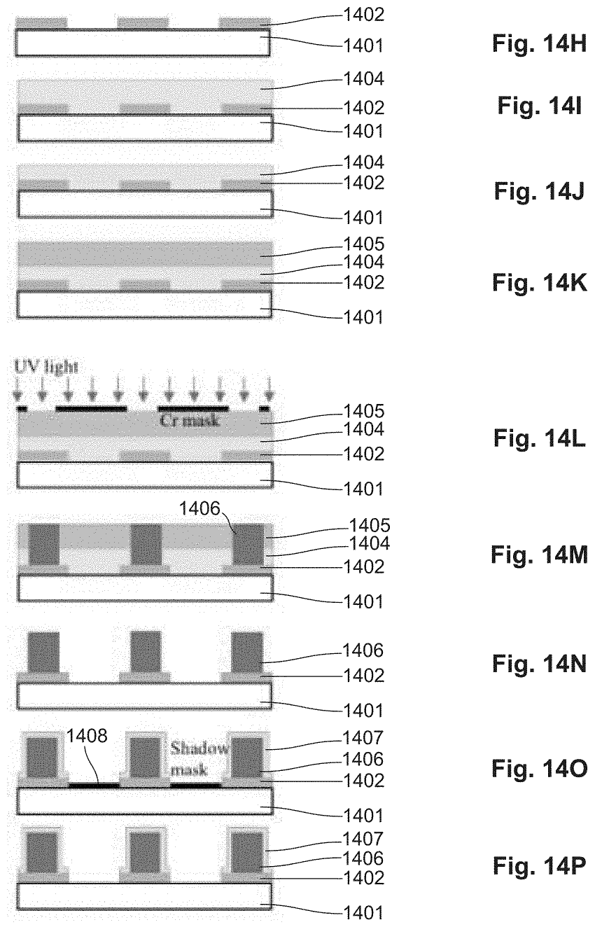

In a first example, shown in FIGS. 14A to 14P, microstructures are made from an insulating polymer applied to a substrate, with electrodes patterned on the substrate through selective etching to act acting as electrical connections for the polymer microstructures. It will be also be appreciated that conductive polymers could be used, for example through suitable doping of an insulating polymer.

In this example, a first step shown in FIGS. 14A to 14G is to selectively pattern an electrode architecture onto a flexible polyethylene terephthalate (PET) substrate 1401. An electrode design, upon which microstructures were to be defined, was patterned on the PET; in this case Indium Tin Oxide (ITO) 1402 layer deposited atop flexible PET substrate, and the electrode pattern selectively etched from the ITO layer. The substrate was prepared (FIG. 14A), before a positive photoresist, AZ1518 (MicroChemicals), was patterned on top of the ITO via photolithography (FIG. 14B), and soft baked (FIG. 14C). The photoresist is selectively exposed to UV (FIG. 14D) to define an electrode pattern, before the photoresist is baked and developed using a developer AZ 726MIF (MicroChemicals) (FIG. 14E) and the exposed ITO regions wet acid etched (FIG. 14F). The photoresist was removed to reveal the final etched ITO pattern that provides the conductive electrodes for the device (FIG. 14G).

In a second step, shown in FIGS. 14H to 14P, 3D microstructures were fabricated from photosensitive polymers onto the ITO electrodes. The patterned PET substrate with ITO electrodes was treated with an oxygen plasma (FIG. 14H), to improve wetting and resist adhesion, and a seed adhesion layer of SU-8 3005 (MicroChemicals) 1404 was spin-coated on to the ITO-PET substrate (FIG. 14I). After baking of the seed SU-8 layer lamination (FIG. 14J) an SUEX SU-8 film resist 1405 (DJ MicroLaminates) was bonded to the substrate (FIG. 14K) through thermal lamination. After alignment and exposure to UV through a mask aligner (FIG. 14L), the exposed SU-8 areas crosslinked to form rows of rectangular microstructures 1406 with vertical wall profile along the conductive ITO fingers 1402 (FIG. 14M). The structures are baked, with the SU-8 1404 and SUEX 1405 before being developed in PGMEA (Propylene glycol monomethyl ether acetate) (Sigma Aldrich), and then hard baked (FIG. 14N). A shadow mask 1408 is applied to the substrate 1401 with the microstructures 1406 being coated with gold 1407 (FIG. 14O) through selective deposition, before the mask is removed (FIG. 14P), leaving selectively metallized microstructures that act as electrodes.

In this example the microstructures have flat tips, but it will be appreciated that other UV lithography techniques such as greyscale lithography, backside diffraction lithography, 2 photon lithography etc. could be employed to define tapered microstructures.

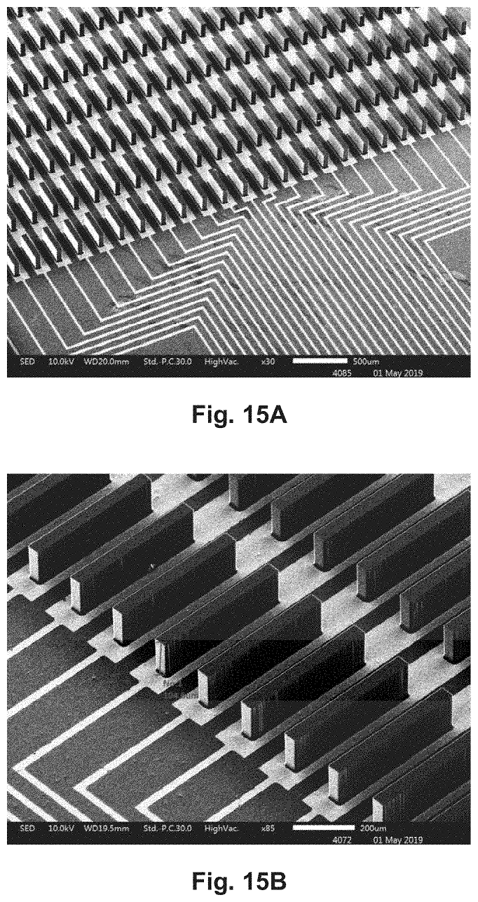

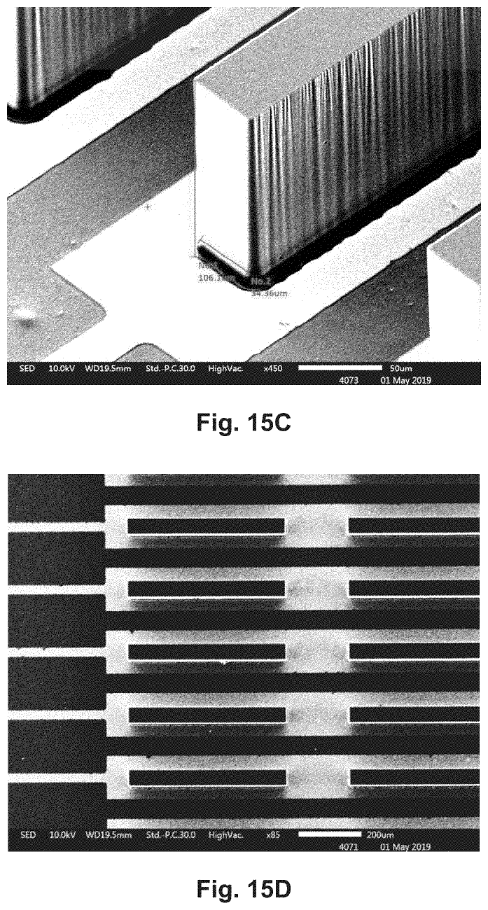

Resulting microstructures are shown in FIGS. 15A to 15D.

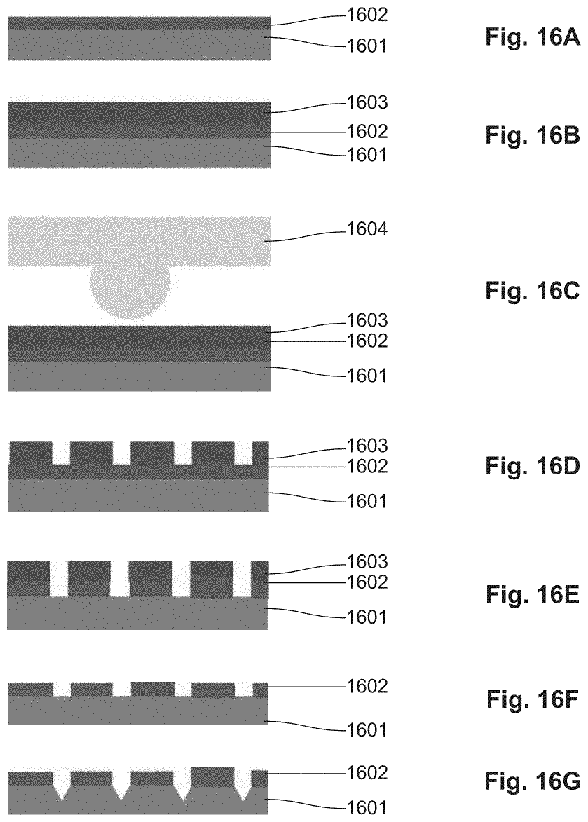

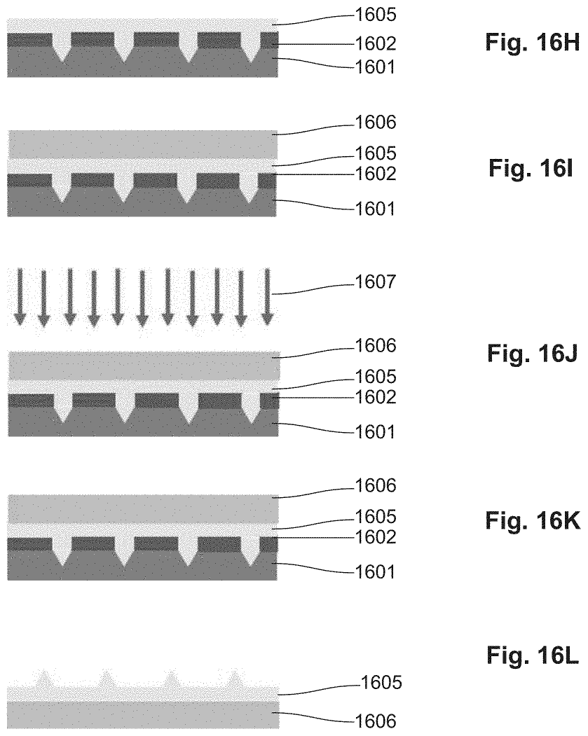

In a second example, shown in FIGS. 16A to 16L, microstructures are made by molding.

In this example, a silicon wafer 1601 was deposited with a 90 nm layer 1602 of Nitride (FIG. 16A). AZ1505 (MicroChemicals) positive resist 1603 was then spun on at 4000 rpm (FIG. 16B). Rectangular pattern to define the blade outline was directly written using a mask writer 1604 (FIG. 16C). The written pattern was developed using AZ 726 MIF (MicroChemicals) for 30 secs (FIG. 16D). Reactive ion etching is used to remove the nitride layer 1602 (FIG. 16F), before the photoresist 1613 is removed (FIG. 1616E). The wafer is then held vertically in a bath of Potassium Hydroxide at 80° C. for 40 mins, to etch the silicon wafer along the crystal axis of the wafer (FIG. 16G). The etching stops at the axis 111 thus defining the sharp tips needed, this then acts as a mold for the devices that are fabricated.

Omni-Coat is used as a lift off resist and is coated onto the wafer to a thickness of about 20 nm, using a spin recipe of 3000 RPM for 1 min and then baking at 200° C. for 1 min. Following this a 5 micron layer 1605 of SU8 3005 is spun on to the wafer at 3000 RPM following by baking at 65° C. for 1 min, then at 95° C. for 20 secs followed by 65° C. again for 1 min (FIG. 16H). The thinner formulation of the SU8 3005 would allow it to flow more easily into the sharp triangular crevices etched into the silicon wafer mold. A layer 2016 of SU8 1600 is then spun on top of this layer to a thickness of 200 microns using a spin recipe of 2000 RPM for 60 secs (FIG. 16I). Following this the wafer was baked at 65° C. for 5 mins, then at 95° C. for 35 mins and then again at 65° C. for 5 mins. This layer of SU8 1600 would allow the sharp tips to stand on a solid layer.

Finally the wafer is flood exposed using an Ultra Violet source 1607 delivering 15 mW/cm² of Power for 40 secs (FIG. 16J). The structures are released by soaking the wafer in an AZ 726 developer solution overnight (FIG. 16K) and exposed the wafer to a thermal shock of 120° C. for 15 secs. The structures are removed from the mold flipped and dried using Nitrogen gas (FIG. 16L).

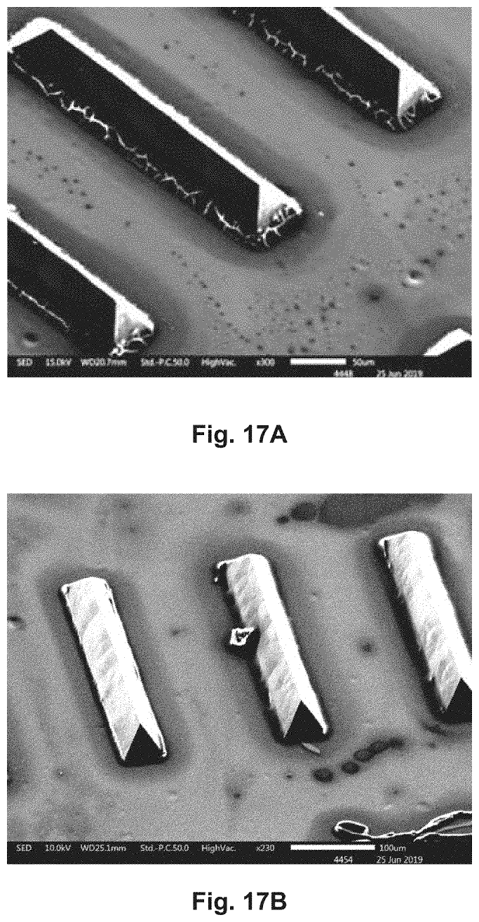

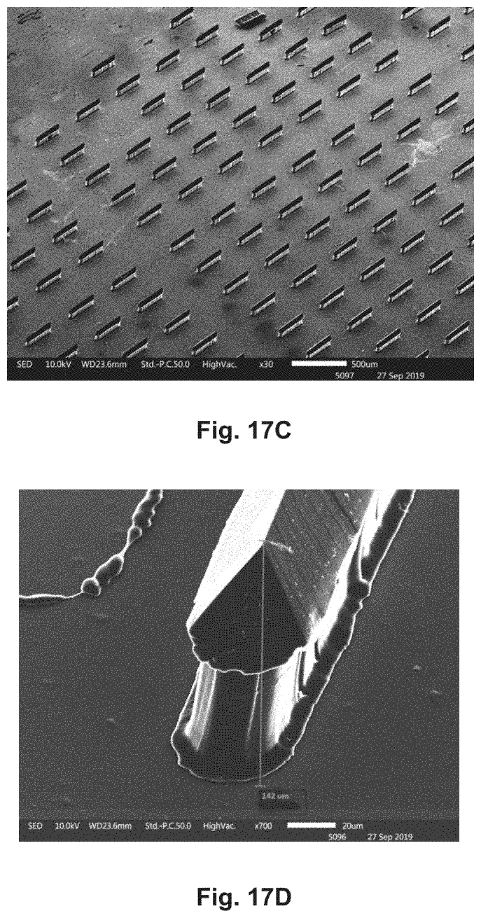

Resulting microstructures are shown in FIGS. 17A and 17B and 17C and 17D.

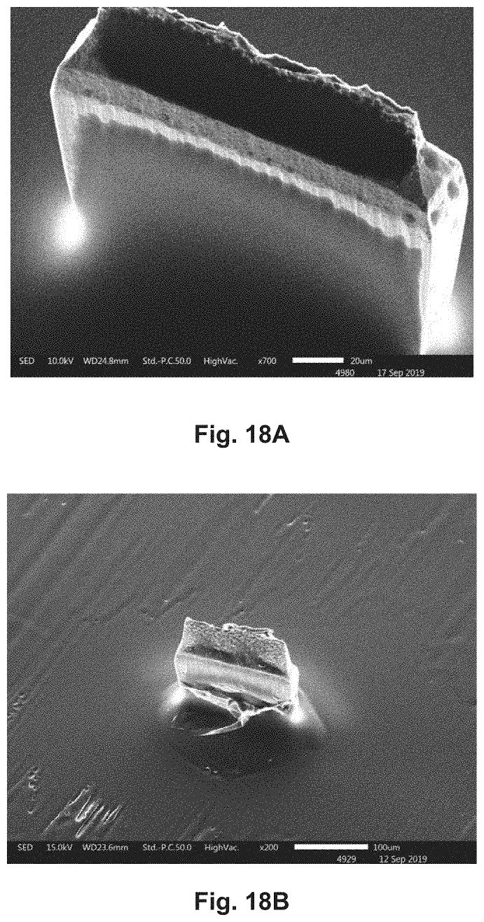

FIGS. 18A and 18B show silicon blades fabricated via etching. FIG. 18A shows the blade coated with a nearly 1 micron thick layer of SU8 3005 which has been diluted in a ratio of 3:2 using SU8 thinner and spun at 5000 RPM for 40 secs. FIG. 18B gives a depiction of the blade selectively coated at its base with the polymer coating. While the tip of the blade is bare and available for detection purposes only at this area. This selective coating is achieved by pressing and removing the coated blade in FIG. 18A into a thin layer of Aluminium foil which mechanically removes the resist from the tip of the blade. This allows the blade to be partially covered with an insulative coating, so that only the tip portion acts as an electrode, thereby allowing measurements to be performed in the epidermis and/or dermis, as described above.

### Optical Sensing

As mentioned above, in some examples, optical sensing can be performed. In this regard, Resazurin is a widely used colorimetric and fluorometric indicator of the metabolic capacity of live cells. At physiological pH resazurin is a deep blue colour, however in the presence of small molecules, like NADH from the mitochondria, resazurin can be reduced to resorufin, which is pink and fluorescent. This assay is quantifiable, inexpensive, and highly sensitive to cellular activity.

To produce optically clear flat-topped microstructures for this proof of concept an etched ITO circuit on a PET Substrate was bonded to an SU8 laminate, which is then patterned using photolithography. There were then coated these structures with biocompatible alginate hydrogel as a vehicle to contain the resazurin.

The alginate hydrogel was prepared by coating bare SU8 microstructures via drop casting: first a poly-1-lysine solution to increase adhesion, then a resazurin/alginate/sucrose hydrogel solution, then a calcium chloride crosslinking solution.

The cells used to reduce the resazurin were dried yeast in a phosphate buffered saline with glucose, which was pipetted on to the microstructures and held at a temperature of 37° C. for 30 minutes. The solution was then pipetted off and the microstructures rinsed and dried before UV-vis measurements.

The resazurin-coated clear microstructures reacted with the cell broth and changed appreciably to a pink colour. UV-vis measurements were taken through the microstructures from 900-300 nm. Photos were taken of representative hydrogel-coated microstructures directly on as well as at a slight angle so that the coating on the sides of the microstructures is visible.

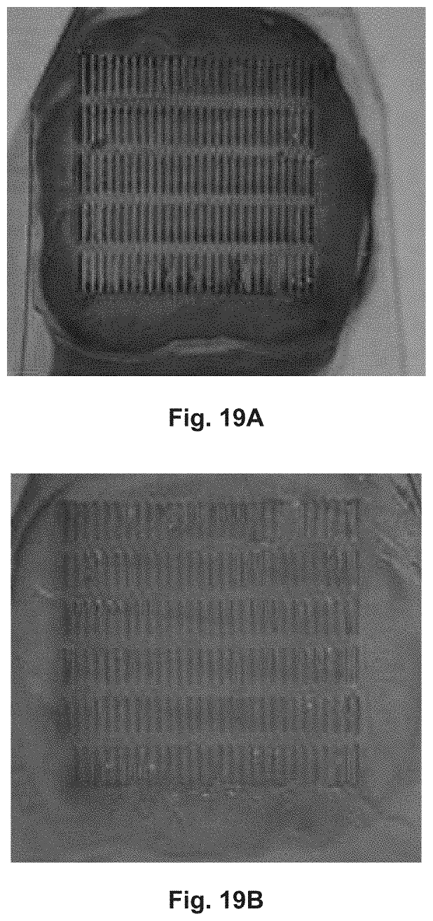

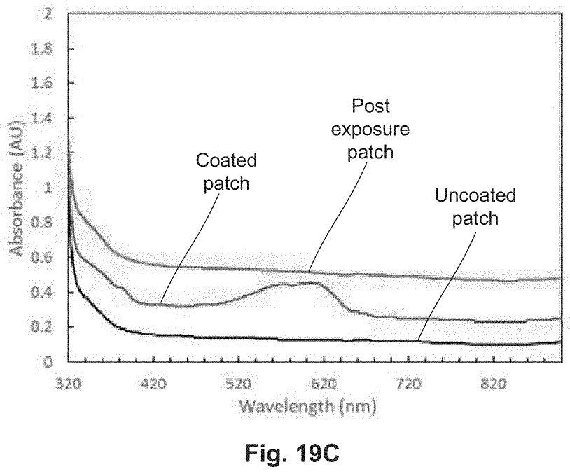

FIG. 19A shows as-deposited resazurin hydrogel on SU8 microstructures, showing the vivid blue colour and distribution of the hydrogel on the sides of the flat-topped fins. FIG. 19B demonstrates the colour change after 30 mins of exposure to the yeast broth. Each microstructure is approximately 200 μm long. The graph in FIG. 19C shows the UV-vis spectra of the blank patch, resazurin-coated patch, and the resazurin-patch post exposure with the lower wavelengths excluded due to high absorption from the SU8. Disappearance of the peaks at 570 nm and 610 nm in the UV-vis spectrum also indicate the reaction with metabolites.

These results demonstrate the ability to detect colour changes in a coating on the patch either using suitable sensing, such as a CCD sensor, photodiode, or similar, or by way of visual inspection.

A further experiment on optical detection of biological analytes was demonstrated by the color changes of electropolymerized polyaniline in the presence of iron (III). Almost 70 percent of iron in the body is found in the red blood cells. Presence of Iron in the ISF may suggest occurrence of hemorrhaging.

In this example, polyaniline changes color when exposed to acids or bases as a result from the changes in its structure when protonated to deprotonate. The structure of PANI also changes when reduced (Leucoemeraldine structure) or oxidized (Emeraldine structure), and so a biologically relevant analyte that can cause redox reaction may be detected from the changes in the color of polyaniline. In this example, Iron (III) ions are deficient of electrons which can be supplemented by another species in the reaction. This transfer of electrons causes oxidation of another reactant which in this case is the polyaniline.

Polyaniline was prepared by electropolymerisation on Gold plated glass substrate. The polymerizing solution contained: 0.1M Aniline in 0.1M HCl. Polyaniline was formed by applying voltage sweep from −0.2 to 1V, at 50 mV/s for 5 and 10 cycles. 5 cycles made a yellow (Leucoemeraldine) polyaniline coating, while 10 cycles made a green (Emeraldine)polyaniline coating.

Optical sensing experiment using acid (0.1 M HCl), base (0.1 M NaOH), and Fe³⁺ (0.1 M FeCl₃) were performed which demonstrated colour changes in Polyaniline. The polyaniline was yellow in acid and was dark blue in base. This color change was reversible. When introduced to Fe3+, the polyaniline turned from greenish to dark blue which was irreversible. The change of color indicated the detection of Fe3+.

### Erythema

Studies have been performed to evaluate the tolerability and functionality of microstructure patches in humans.

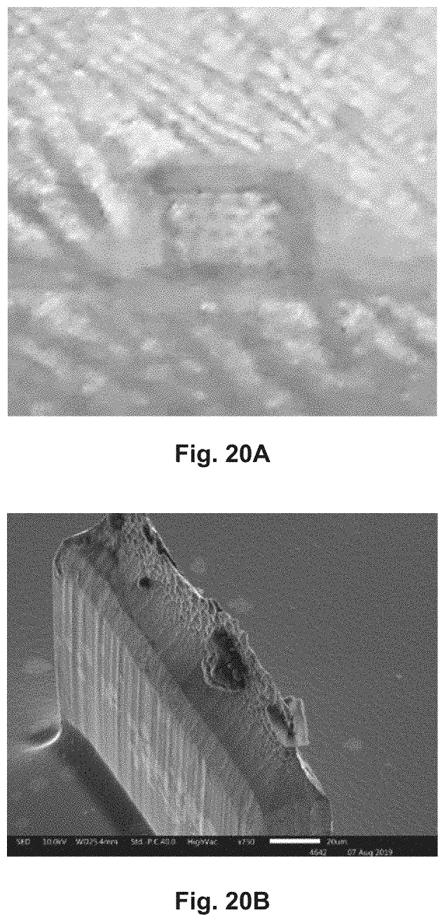

In one example, a qualitative tolerability assessment was performed following microstructure patches application which noted a very mild local response at the application site immediately post-removal. This was characterized by slight indentation with no overt erythema or oedema, which was resolved within 15 minutes of removal. This is shown in FIG. 20A. This shows the indentation was most prominent around the edges and corners of the microstructure patch, with very mild redness at these locations, and with no redness associated with the microstructures themselves.

Scanning Electron Microscopy (SEM) was performed to confirm that the microstructures had, in fact, penetrated the skin, showing cellular debris remaining on the removed microstructures, as shown in FIG. 20B, confirming successful microstructure penetration despite the absence of overt erythema.

To investigate this observation further, we two dedicated erythema studies were performed with multiple subjects. These studies investigated the local skin response to microstructure patch application to the skin of the anterior forearm over a time period of 2 hours. Microstructure patches were applied using a guided load cell mechanism, at a force of either 5N remaining in place for 30 minutes (Study 1) or 3N and remaining in place for 10 minutes (Study 2).

The first human erythema study was on five volunteers. In some cases, hair was removed from the skin using depilatory cream and a paper mask was fixed to the application area to avoid any effect due to sensitivity to surgical adhesives in tapes. Three separate non-functionalised microstructure patches were applied to skin exposed by windows in the paper mask, and a fourth window was untreated and used as a control for comparison.

Observations were made for local erythema and a scoring rubric was used as given in Table 5 below.

TABLE 5

| eScore | Observation |
|---|---|
| 0 | No discernable difference relative to |
| | control |
| 1 | Very mild redness |
| 2 | Mild redness |
| 3 | Red region extending beyond 4 mm² |
| | application area |
| 4 | Extensive redness and/or capillary |
| | rupture |
| 5 | Frank blood and/or oedema superficially |

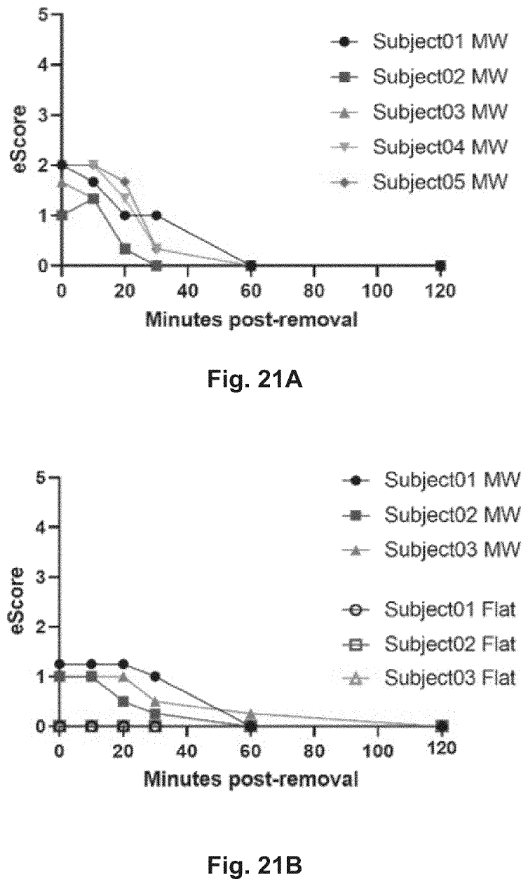

Results from the first study are shown in FIG. 21A, which shows the eScores for Subjects 01-05 in this study, which were independently assessed at 10, 20, 30, 60 and 120 minutes post-application. Data points represent the average eScore from three Microwearables per subject per timepoint.

Results show that all volunteers experienced some mild or very mild erythema at the site of Microwearable application as observed immediately after removal, which quickly resolved within 60 minutes. No erythema was noted after this time point. Similar to the earlier single subject observation, the indentation/redness was localised around the edges of the Microwearable, with little or no effect seem from the microstructures themselves.

The second erythema study was performed on three volunteers. Two Microwearable devices were applied at 3N and were removed after 10 minutes of wearing. To investigate further the ‘edge effect’ observed in a first-in-human trial and in Study 1, a flat patch (i.e. without microstructures) was applied on the third skin site, for comparison. The fourth window remained untreated as a control. Results are shown in FIG. 21B, which shows the eScore observations (data points are an average of 2 separate observations per subject per time point) over 120 minutes post-removal.

Results are similar to Study 1 in that no subject experienced erythema more extensive than ‘mild redness’ at the site immediately prior to removal of the Microwearable. This mild erythema resolved quickly within 60 minutes, with one subject with a score of 0.5 at 60 minutes, which subsequently resolved completely by 120 minutes. No erythema was observed following application of flat patches, which may suggest that the very mild/mild erythema observed following microstructure patch application is associated with skin barrier penetration (i.e. by the presence of microstructures).

Microstructure patch eScores were, in general, lower in Study 2 than Study 1, suggesting that lowering the application force of application reduces the extent of the mild erythema that occurs. As the erythema was observed immediately after the microstructure patches were removed and did not increase over time, it appears erythema is caused by the application event itself—driven by the corners and edges of the microstructure patches—and is not exacerbated by continuous wearing. Future-generation microstructure patch can use different edges and corner configurations leading to negligible erythema.

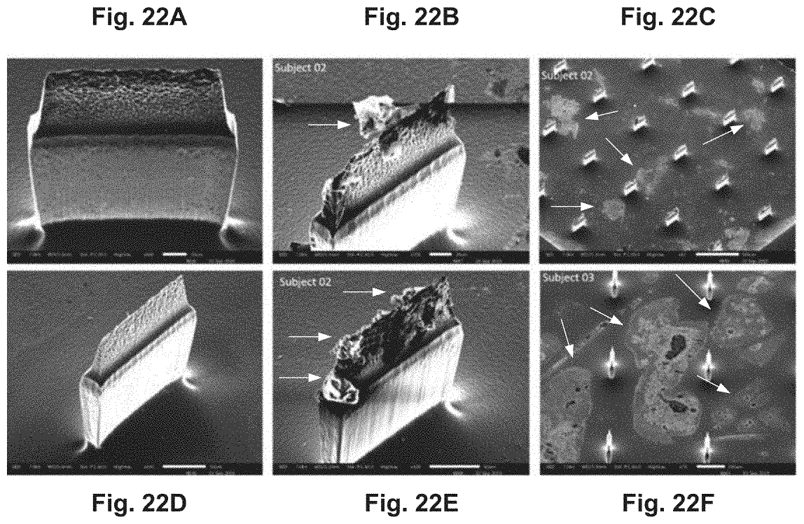

As no local erythema was observed within the area covered by microstructures, SEM was performed to confirm that the structures had successfully penetrated the skin of the subjects in Study 1. Example images of individual or row of microstructures after application to two subjects are shown in FIG. 22, including images of individual microstructures prior to application to the skin (FIGS. 22A and 22D) and images post application (FIGS. 22B, 22C and 22E, 22F).

Images from all subjects confirmed successful penetration of the skin, from the presence of biological material located on the upper portion of the microstructures (FIGS. 22B and 22E), with arrows indicating examples of cellular debris extracted by the microstructures on removal.

FIGS. 22C and 22F show rows of microstructures, and exhibit areas with dried interstitial fluid as indicated by the arrows. These observations confirm that the microstructures have successfully breached the outermost stratum corneum layer of the skin and are able to access cellular environments beneath to gain access to the interstitial fluid, which is the source of bio-signals including biomarkers of disease.

It is therefore apparent that microstructure patches are at worst only associated with very mild/mild erythema at the site of application. This mild local response is transient, and is completely resolved within 60-120 mins post-application. Any redness immediately occurs after application, and is not associated with continuous wearing of the microstructure patch.

Any erythema is focused around the edges and corners of the microstructure patch, with little/no erythema noted in the area covered by microstructures, but the observation that a flat patch had no effect suggests that the erythema after microstructure patch application is associated with a physical breach of the skin barrier.

Despite the observation that microstructures did not cause overt erythema, it was we confirmed that microstructure penetration was successful, with visible breaching of the stratum corneum and with confirmed access to skin compartments rich in interstitial fluid.

### Use of the System

The system of the invention may be used to determine the presence, absence, level or concentration of one or more analytes in a wide range of applications as discussed herein, including, diagnosing or monitoring the progression of a disease, disorder or condition in a subject; the presence, absence, level or concentration of an illicit substance or non-illicit substance, or a chemical warfare agent, poison or toxin, or the level or concentration of a medicament.

Accordingly, in a further aspect, there is provided a method for diagnosing or monitoring the progression of a disease, disorder or condition in a subject, comprising determining the presence, absence, level or concentration of one or more analytes in the viable epidermis and/or dermis of the subject using the system of the invention, and determining the presence, absence and/or progression of the disease, disorder or condition based on whether the one or more analytes is present or absent, or whether the level or concentration of the one or more analytes is above or below a corresponding predetermined threshold that correlates with the presence, absence or progression of the disease, disorder or condition.

The invention also provides the use of the system of the invention for diagnosing or monitoring the progression of a disease, disorder or condition in a subject. There is further provided the system of the invention for use in diagnosing or monitoring the progression of a disease, disorder or condition in a subject. In particular embodiments of any one of the above aspects, the system determines the presence, absence, level or concentration of one or more analytes in the viable epidermis and/or dermis of the subject and the presence, absence and/or progression of the disease, disorder or condition is determined based on whether the one or more analytes is present or absent, or whether the level or concentration of the one or more analytes is above or below a corresponding predetermined threshold that correlates with the presence, absence or progression of the disease, disorder or condition.

Suitable diseases, disorders or conditions, analytes and exemplary concentration levels are discussed supra.

In some embodiments, the disease, disorder or condition is selected from cardiac damage, myocardial infarction and acute coronary syndrome, and the one or more analytes is troponin or a subunit thereof. In particular embodiments, the one or more analytes is troponin I.

In another aspect, there is provided a method of treating a disease, disorder or condition in a subject comprising determining the presence, absence, level or concentration of one or more analytes in the viable epidermis and/or dermis of the subject using the system of the invention, determining the presence or progression of the disease, disorder or condition based on whether the one or more analytes is present, or whether the level or concentration of the one or more analytes is above or below a corresponding predetermined threshold that correlates with the presence or progression of the disease, disorder or condition, and administering a treatment for the disease, disorder or condition.

In a further aspect, there is provided a method of treating a disease, disorder or condition in a subject comprising exposing the subject to a treatment regimen for treating the disease, disorder or condition based on an indicator obtained from an indicator-determining method, said indicator-determining method comprising determining the presence, absence, level or concentration of one or more analytes in the viable epidermis and/or dermis of the subject using the system of the invention, and determining the presence or progression of the disease, disorder or condition based on whether the one or more analytes is present, or whether the level or concentration of the one or more analytes is above or below a corresponding predetermined threshold that correlates with the presence or progression of the disease, disorder or condition.

In a related aspect, the present invention provides a method for managing a disease, disorder or condition in a subject comprising exposing the subject to a treatment regimen for treating the disease, disorder or condition based on an indicator obtained from an indicator-determining method, said indicator-determining method comprising determining the presence, absence, level or concentration of one or more analytes in the viable epidermis and/or dermis of the subject using the system of the invention, and determining the presence or progression of the disease, disorder or condition based on whether the one or more analytes is present, or whether the level or concentration of the one or more analytes is above or below a corresponding predetermined threshold that correlates with the presence or progression of the disease, disorder or condition.

In any one of the above aspects, the predetermined threshold represents a level or concentration of the analyte in a corresponding sample from a control subject (e.g. in the viable epidermis and/or dermis of the control subject), or represents a level or concentration above or below the level or concentration of the analyte in a corresponding sample from a control subject, and levels or concentrations above or below said threshold indicates the presence, absence or progression of a disease, disorder or condition. The control subject may be a subject who does not have the disease, disorder or condition; a subject who does have the disease, disorder or condition; or a subject who has a particular stage or severity of the disease, disorder or condition. When progression of the disease, disorder or condition is being monitored, the predetermined threshold may be a level or concentration of the analyte in a sample from the same subject taken at an earlier time (e.g. several minutes, hours, days, weeks or months earlier), and an increase or decrease in the analyte level or concentration may indicate the progression or regression of the disease, disorder or condition.

Suitable treatments for the disease, disorders or conditions discussed supra are well known in the art, and a skilled person will readily be able to select an appropriate treatment. For example, suitable disorders and exemplary treatments include, but are not limited to, renal failure and treatment with dialysis, a kidney transplant, an angiotensin-converting enzyme inhibitor (e.g. benazepril, zofenopril, perindopril, trandolapril, captopril, enalapril, lisinopril or ramipril), an angiotensin II receptor blocker (e.g. losartan, irbesartan, valsartan, candesartan, telmisartan or fimasartan), a diuretic (e.g. furosemide, bumetanide, ethacrynic acid, torsemide, chlorothiazide, hydrochlorothiazide, bendroflumethiazide or trichlormethiazide), a statin (e.g. atorvastatin, fluvastatin, lovastatin, mevastatin, pitavastatin, pravastatin, rosuvastatin or simvastatin), calcium, glucose or sodium polystyrene sulfonate, and/or a calcium infusion; cardiac failure and treatment with an angiotensin-converting enzyme inhibitor (e.g. benazepril, zofenopril, perindopril, trandolapril, captopril, enalapril, lisinopril or ramipril), an angiotensin II receptor blocker (e.g. losartan, irbesartan, valsartan, candesartan, telmisartan or fimasartan), a diuretic (e.g. furosemide, bumetanide, ethacrynic acid, torsemide, chlorothiazide, hydrochlorothiazide, bendroflumethiazide or trichlormethiazide), a beta blocker (e.g. carvedilol, metoprolol or bisoprolol), an aldosterone antagonist (e.g. spironolactone or eplerenone), and/or an inotrope (e.g. digoxin, berberine, levosimendan, calcium, dopamine, dobutamine, dopexamine, epinephrine, isoprenaline, norepinephrine, angiotensin II, enoximone, milrinone, amrinone, theophylline, glucagon or insulin); essential hypertension and treatment with a beta blocker (e.g. carvedilol, metoprolol or bisoprolol), a calcium channel blocker (e.g. amlodipine, felodipine, isradipine, nicardipine, nifedipine, nimodipine or nitrendipine), a diuretic (e.g. furosemide, bumetanide, ethacrynic acid, torsemide, chlorothiazide, hydrochlorothiazide, bendroflumethiazide or trichlormethiazide), angiotensin-converting enzyme inhibitor (e.g. benazepril, zofenopril, perindopril, trandolapril, captopril, enalapril, lisinopril or ramipril), an angiotensin II receptor blocker (e.g. losartan, irbesartan, valsartan, candesartan, telmisartan or fimasartan), and/or a renin inhibitor (e.g. aliskiren); bacterial infection and treatment with antibiotics (e.g. quinolones (e.g. amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin, gemifloxacin, or garenoxacin), tetracyclines, glycylcyclines or oxazolidinones (e.g. chlortetracycline, demeclocycline, doxycycline, lymecycline, methacycline, minocycline, oxytetracycline, tetracycline, tigecycline, linezolide or eperezolid), aminoglycosides (e.g. amikacin, arbekacin, butirosin, dibekacin, fortimicins, gentamicin, kanamycin, menomycin, netilmicin, ribostamycin, sisomicin, spectinomycin, streptomycin or tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, cefdinir, ceftibuten or cefozopran), rifamycins, macrolides (e.g. azithromycin, clarithromycin, erythromycin, oleandomycin, rokitamycin, rosaramicin, roxithromycin or troleandomycin), ketolides (e.g. telithromycin or cethromycin), coumermycins, lincosamides (e.g. clindamycin or lincomycin) or chloramphenicol); viral infection and treatment with antivirals (e.g. abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanamivir or zidovudine); autoimmune disorders and treatment with immunosuppressants (e.g. prednisone, dexamethasone, hydrocortisone, budesonide, prednisolone, tofacitinib, cyclosporine, cyclophosphamide, nitrosoureas, platinum compounds, methotrexate, azathioprine, mercaptopurine, fluorouracil, dactinomycin, anthracyclines, mitomycin C, bleomycin, mithramycin, antithymocyte globulin, thymoglobulin, Muromonab-CD3, basiliximab, daclizumab, tacrolimus, sirolimus, everolimus, infliximab, etanercept, mycophenolic acid or mycophenolate, fingolimod, azathioprine, leflunomide, abatacept, adalimumab, anakinra, certolizumab, golimumab, ixekizumab, natalizumab, rituximab, secukinumab, toclizumab, ustekinumab, vedolizumab or myriocin) and/or NSAIDs (e.g. acetylsalicylic acid (aspirin), diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin, zomepirac, celecoxib, deracoxib, etoricoxib, mavacoxib or parecoxib); rheumatological disorders and treatment with NSAIDs as described supra, DMARDs (e.g. methotrexate, hydroxychloroquinone or penicillamine), prednisone, dexamethasone, hydrocortisone, budesonide, prednisolone, etanercept, golimumab, infliximab, adalimumab, anakinra, rituximab, abatacept, and/or other immunosuppressants described supra; sepsis and antibiotics as described supra, immunosuppressants as described supra and/or an antihypotensive agent (e.g. vasopressin, norepinephrine, dopamine or epinephrine); and pulmonary embolism and treatment with an anticoagulant (e.g. heparin, warfarin, bivalirudin, dalteparin, enoxaparin, dabigatran, edoxaban, rivaroxaban, apixaban or fondaparinux) and/or a thrombolytic/fibrinolytic (e.g. tissue plasminogen activator, reteplase, streptokinase or tenecteplase).

In some embodiments, the disease, disorder or condition is cardiac damage, myocardial infarction or acute coronary syndrome, the one or more analytes is troponin or a subunit thereof. Suitable treatments for cardiac damage, myocardial infarction or acute coronary syndrome may include, but are not limited to, aspirin, an anticoagulant (e.g. heparin, warfarin, bivalirudin, dalteparin, enoxaparin dabigatran, edoxaban, rivaroxaban, apixaban or fondaparinux), a beta-blocker (e.g. carvedilol or metoprolol), a thrombolytic/fibrinolytic (e.g. tissue plasminogen activator, reteplase, streptokinase or tenecteplase), an angiotensin-converting enzyme inhibitor (e.g. benazepril, zofenopril, perindopril, trandolapril, captopril, enalapril, lisinopril or ramipril), an angiotensin II receptor blocker (e.g. losartan, irbesartan, valsartan, candesartan, telmisartan or fimasartan), a statin (e.g. atorvastatin, fluvastatin, lovastatin, mevastatin, pitavastatin, pravastatin, rosuvastatin or simvastatin), an analgesic (e.g. morphine, etc.), nitroglycerin, and the like, or combinations thereof.

The invention further contemplates the use of the system of the invention for determining the presence, absence, level or concentration of an illicit substance or non-illicit substance of abuse in a subject. Accordingly, in another aspect, there is provided a method of determining the presence, absence, level or concentration of an illicit substance or non-illicit substance of abuse in a subject, comprising determining the presence, absence, level or concentration of the illicit substance, non-illicit substance of abuse or a metabolite thereof in the viable epidermis and/or dermis of the subject using the system of the invention.

There is also provided the use of the system of the invention for determining the presence, absence, level or concentration of an illicit substance or non-illicit substance of abuse in a subject, and the system of the invention for use in determining the presence, absence, level or concentration of an illicit substance or non-illicit substance of abuse in a subject. In particular embodiments of any one of these aspects, the system determines the presence, absence, level or concentration of the illicit substance, non-illicit substance of abuse or metabolite thereof in the viable epidermis and/or dermis of the subject.

Suitable illicit substances are discussed supra and include, but are not limited to, methamphetamine, amphetamine, 3,4-methylenedioxymethamphetamine (MDMA), N-ethyl-3,4-methylenedioxyamphetamine (MDEA), 3,4-methylenedioxy-amphetamine (MDA), cannabinoids (e.g. delta-9-tetrahydrocannabinol, 11-hydroxy-delta-9-tetrahydrocannabinol, 11-nor-9-carboxydelta-9-tetrahydrocannabinol), cocaine, benzoylecgonine, ecgonine methyl ester, cocaethylene, ketamine, and the opiates (e.g. heroin, 6-monoacetylmorphine, morphine, codeine, methadone and dihydrocodeine). Non-limiting non-illicit substances of abuse include alcohol, nicotine, prescription medicine or over the counter medicine taken for non-medical reasons, a substance taken for a medical effect, wherein the consumption has become excessive or inappropriate (e.g. pain medications, sleep aids, anti-anxiety medication, methylphenidate, erectile-dysfunction medications), and the like.

The invention further contemplates the use of the system of the invention for determining the presence, absence, level or concentration of a chemical warfare agent, poison and/or toxin in a subject. Accordingly, in another aspect, there is provided a method of determining the presence, absence, level or concentration of a chemical warfare agent, poison and/or toxin in a subject, comprising determining the presence, absence, level or concentration of the chemical warfare agent, poison and/or toxin or a metabolite thereof in the viable epidermis and/or dermis of the subject using the system of the invention. In particular embodiments, the method is for determining the presence, absence, level or concentration of a chemical warfare agent.

There is also provided the use of the system of the invention for determining the presence, absence, level or concentration of a chemical warfare agent, poison and/or toxin in a subject, and the system of the invention for use in determining the presence, absence, level or concentration of a chemical warfare agent, poison and/or toxin in a subject; especially a chemical warfare agent. In particular embodiments of any one of these aspects, the system determines the presence, absence, level or concentration of the chemical warfare agent, poison and/or toxin or a metabolite thereof in the viable epidermis and/or dermis of the subject.

Suitable chemical warfare agents, poisons and/or toxins are discussed supra.

The system of the invention may also be used to determine and/or monitor the level or concentration of a medicament administered to a subject, for example, to optimise and/or adjust the dose of the medicament. The invention provides a method for determining and/or monitoring the level or concentration of a medicament administered to a subject, comprising determining the level or concentration of the medicament or a component or metabolite thereof in the viable epidermis and/or dermis of the subject using the system of the invention.

There is further provided the use of the system of the invention for determining and/or monitoring the level or concentration of a medicament administered to a subject, and the system of the invention for use in determining and/or monitoring the level or concentration of a medicament administered to a subject. In particular embodiments, the system of the invention determines the level or concentration of the medicament or a component or metabolite thereof in the viable epidermis and/or dermis of the subject.

In some embodiments, the dose of the medicament is increased or decreased following determination of the level or concentration of the medicament or a component or metabolite thereof.

In a further aspect, there is provided a method of monitoring the efficacy of a treatment regimen in a subject with a disease, disorder or condition, wherein the treatment regimen is monitored for efficacy towards a desired health state (e.g. absence of the disease, disorder or condition. Such method generally comprises determining the presence, absence, level or concentration of one or more analytes indicative of the efficacy of the treatment regimen in the viable epidermis and/or dermis of the subject using the system of the invention after treatment of the subject with the treatment regimen, and comparing the level or concentration of the one or more analytes to a reference level or concentration of the one or more analytes which is correlated with a presence, absence or stage of the disease, disorder or condition to thereby determine whether the treatment regimen is effective for changing the health status of the subject to a desired health state. In some embodiments, the one or more analytes is a medicament administered during the treatment regimen, or a component or metabolite thereof.

In a related aspect, there is provided a method of monitoring the efficacy of a treatment regimen in a subject with a disease, disorder or condition, wherein the treatment regimen is monitored for efficacy towards a desired health state (e.g. absence of the disease, disorder or condition). Such method generally comprises determining an indicator according to an indicator-determining method, said indicator-determining method comprising determining the presence, absence, level or concentration of one or more analytes in the viable epidermis and/or dermis of the subject using the system of the invention after treatment of the subject with the treatment regimen, and assessing the likelihood of the subject having a presence, absence or stage of a disease, disorder or condition based on whether the one or more analytes is present, or whether the level or concentration of the one or more analytes is above or below a corresponding predetermined threshold that correlates with the presence, absence or stage of the disease, disorder or condition, using the indicator to thereby determine whether the treatment regimen is effective for changing the health status of the subject to a desired health state. In some embodiments, the one or more analytes is a medicament administered during the treatment regimen, or a component or metabolite thereof.

In some embodiments of any one of the above aspects, the treatment regimen is adjusted following such methods. Suitable predetermined thresholds for such aspects are discussed supra.

The invention also provides the system of the invention for use in such methods, and the use of the system for such methods.

A skilled person will readily appreciate that the system of the invention may be used to determine and monitor the level or concentration of a wide range of medicaments and treatment regimens and will readily be able to use and select suitable medicaments and treatment regimens. For example, suitable medicaments include, but are not limited to, cancer therapies, vaccines, analgesics, antipsychotics, antibiotics, anticoagulants, antidepressants, antivirals, sedatives, antidiabetics, contraceptives, immunosuppressants, antifungals, antihelmintics, stimulants, biological response modifiers, NSAIDs, corticosteroids, DMARDs, anabolic steroids, antacids, antiarrhythmics, thrombolytics, anticonvulsants, antidiarrheals, antiemetics, antihistamines, antihypertensives, anti-inflammatories, antineoplastics, antipyretics, antivirals, barbiturates, β-blockers, bronchodilators, cough suppressants, cytotoxics, decongestants, diuretics, expectorants, hormones, laxatives, muscle relaxants, vasodilators, tranquilizers and vitamins.

In particular embodiments, the medicament is one which has a narrow therapeutic window, such as particular antibiotics (e.g. aminoglycosides including kanamycin, gentamycin and streptomycin), anticonvulsants (e.g. carbamazepine and clonazepam), vasodilators, anticoagulants including heparin and warfarin, digoxin, and the like. In such embodiments, the methods and uses may further comprise increasing or decreasing the dose of the medicament administered to the subject.

In any one of the above aspects, the methods and uses further comprise attaching the system of the invention to the skin of the subject prior to determining the presence, absence, level or concentration of the one or more analytes. In such embodiments, the system of the invention breaches a stratum corneum of the subject.

Accordingly, the above described arrangement provides a wearable monitoring device that uses microstructures that breach a barrier, such as penetrating into the stratum corneum in order to perform measurements on a subject. The measurements can be of any appropriate form, and can include measuring the presence of biomarkers or other analytes within the subject, measuring electrical signals within the subject, or the like. Measurements can be performed through visual inspection, or can be analysed and used to generate an indicator indicative of a health status of the subject.

In one example, the above described system allows analytes to be detected in specific tissue sites in the skin, in situ. The microstructures can be coated with a reagent or binding agent, allowing analytes within the subject to react with or bind to the microstructures in turn allowing these to be detected using suitable optical or electrical measurement techniques. The coatings can be specifically designed to capture analytes with extremely high specificity. Such specificity allows specific analytes of interest to be detected without the need for purification or complex chemical analysis.

The length of the structures can be controlled during manufacture to enable targeting of specific layers in the target tissue. In one example, this is performed to target analytes in the epidermal and/or dermal ISF, although analytes in capillary blood can also be targeted.

Specific probes can be localized to individual structures or areas of structures, so that multiple targets can be analysed in a single assay simply by their location in a 2-dimensional array. This could facilitate the analysis of disease-specific analyte panels to increase the sensitivity/specificity of the diagnostic results.

The patches can therefore provide a measurement device which overcomes the need for traditional blood or ISF samples to be taken for diagnostic purposes representing an opportunity for a clinician to diagnose and avoid time and processing costs at centralised testing facilities. It may also open new markets since diagnostic equipment and blood sampling expertise is not needed e.g. in developing countries, ‘in-field’ military applications, medical countermeasures, emergency and triage.

This allows patches to be used as a non-invasive, pain-free measurement platform that can measure analytes in situ. The type of material detected by the patch may be controlled by the length of the structures, such that ISF can be targeted specifically. This embodiment does not include a specific analysis type; a number of established techniques can be used for fluid analysis including, but not limited to, mass spectrometry, microarrays, DNA/protein sequencing, HPLC, ELISA, Western Blots and other gel methods, etc.

Using affinity surface coatings on each structure allow a reduction of non-specific adsorption of ISF components whilst facilitating specific extraction of the molecular targets of interest.

By arranging the structures in a two-dimensional format, multiple probes can be attached to the same patch, with the results from the sandwich assay decoded based on the 2-D array position of the individual structures. This essentially allows array-style processing without the need for sample extraction, purification, labelling, etc.

Accordingly, in one example, the above described system provides a minimally-invasive and pain-free way to access blood-borne biomarkers of disease: by accessing the outer skin layers with devices applied to the skin that are also pain-free. Currently, blood is accessed by a needle/lancet which is often painful and laborious. Alternatively, blood is accessed directly in the body by surgically implanting a sensor. Surgical implants are not likely to be used widely, as implanting is an invasive procedure, with limited choice of materials suitable for implantation.

The system can provide rapid “on the spot” disease detection on the person, rather than the delays of sending blood samples to pathology laboratories for processing. This is also an advance over the current point-of-care devices, which usually still require a blood sample (e.g. by a needle) to be analysed away from the body.

The system can provide high-fidelity, low power, low cost body signal (e.g. biopotential, optical) sensing for practical disease/health diagnostics. As one example, pre-clinical animal skin testing of microstructure patches show a 100 fold reduction of bioimpedance, compared to standard, approaches applied to the surface of skin, leading to improved signal to noise ratio.

The system can provide simple, semi-continuous or continuous monitoring: a low cost-device micro wearable would be applied to the skin and potentially be worn for days (or longer), and then simply replaced by another micro wearable component. Thus, micro wearables provide a route for monitoring over time—which can be particularly important in detecting sudden events (e.g. cardiac biomarkers for a heart attack)—without surgically implanting a sensor into the body.

In one example, the above described approach can allow wearables to provide widespread, low-cost healthcare monitoring for a multitude of health conditions that cannot be assayed by current devices, which are placed on the skin.

In one example, the microstructure patches penetrate the skin barrier and so unlike today's wearables, access blood-borne biomarkers of disease for rapid “on the spot” disease detection on the person. Contrast this to the current method of sending blood samples to pathology laboratories for processing. This is also an advance over the current point-of-care devices, which usually still require a blood sample (e.g. by a needle) to be analysed away from the body.

In one example, the system can provide a low-cost microstructure patches would be applied to the skin and potentially be worn for days (or longer) for simple and pain free semi-continuous or continuous monitoring, and then simply replaced by another microstructure patch component. Thus, microstructure patches provide a route for monitoring over time—which can be particularly important in detecting sudden events (e.g. cardiac biomarkers for a heart attack)—without surgically-implanting a sensor into the body.

Embodiment 1. A system for detecting analytes in a biological subject, the system including at least one substrate including one or more microstructures configured to breach a functional barrier of the subject, and wherein the one or more microstructures are responsive to a presence, absence, level or concentration of analytes to cause a change in appearance thereby indicating that a presence, absence, level or concentration of analytes has been detected.

Embodiment 2. A system according to embodiment 1, wherein the functional barrier is at least one of: multiple layers; a mechanical discontinuity; a tissue discontinuity; a cellular discontinuity; a neural barrier; a sensor barrier; a cellular layer; a skin layer; a mucosal layer; an internal barrier; an external barrier; an inner barrier within an organ; an outer barrier of an organ; an epithelial layer; an endothelial layer; a melanin layer; an optical barrier; an electrical barrier; molecular weight barrier; basal layer; and, a stratum corneum.

Embodiment 3. A system according to embodiment 1 or embodiment 2, wherein the change in appearance includes at least one of: a change in visual appearance; a change in pattern; a change in electromagnetic properties; a colour change; a change in hue; a change in saturation; a change in lightness; a change in chroma; a change in colourfulness; a change in colour constancy; a change in radiance; a change in luminance; a change in brightness; a change in fluorescence; a change in photoluminescence; a change in phosphorescence; a change in reflectivity; a change in refractivity; a reversible change in appearance; and, an irreversible change in appearance.

Embodiment 4. A system according to any one of the embodiments 1 to 3, wherein the change in appearance is at least one of: a change in the tissue; a change in the substrate; a change on the one or more microstructures; a change in the one or more microstructures; and, is viewable through the substrate.

Embodiment 5. A system according to any one of the embodiments 1 to 4, wherein at least some of the microstructures include at least one of: surface features that cause a change in appearance of the microstructures; a material that undergoes a change in appearance; and, a coating that causes a change in appearance.

Embodiment 6. A system according to any one of the embodiments 1 to 5, wherein at least some of the microstructures include a coating and wherein the coating at least one of: undergoes a change in appearance in response to a presence, absence, level or concentration of analytes; causes a change in appearance of surrounding tissue in response to a presence, absence, level or concentration of analytes; causes a change in appearance of analytes; and, dissolves at least one of: after a defined time period; in response to a presence, absence, level or concentration of analytes; and, upon breaching or penetration of the functional barrier.

Embodiment 7. A system according to embodiment 5 or embodiment 6, wherein at least one of: at least some microstructures are uncoated; at least some microstructures are porous with an internal coating; at least some microstructures are partially coated; different microstructures have different coatings; different parts of microstructures include different coatings; and, at least some microstructures include multiple coatings.

Embodiment 8. A system according to any one of the embodiments 5 to 7, wherein the coating at least one of: undergoes a shape change to selectively anchor microstructures; modifies surface properties to at least one of: increase hydrophilicity; increase hydrophobicity; and, minimize biofouling; attracts at least one substance to the microstructures; repels at least one substance from the microstructures; provides a physical structure to at least one of: facilitate penetration of the barrier; strengthen the microstructures; and, anchor the microstructures in the subject; dissolves to at least one of: expose a microstructure; expose a further coating; and, expose a material; provides stimulation to the subject; contains a material; and, selectively releases a material.

Embodiment 9. A system according to any one of the embodiments 1 to 8, wherein at least some of the microstructures include a material including at least one of: a bioactive material; a reagent for reacting with analytes in the subject; a binding agent for binding with analytes of interest; a probe for selectively targeting analytes of interest; a material to reduce biofouling; a material to attract at least one substance to the microstructures; a material to repel at least one substance from the microstructures; a material to attract at least some analytes to the projections; and, a material to repel at least some analytes from the projections.

Embodiment 10. A system according to any one of the embodiments 1 to 9, wherein the substrate includes a plurality of microstructures and wherein different microstructures are at least one of: differentially responsive to analytes; responsive to different analytes; responsive to different combination of analytes; and, responsive to different concentrations of analytes.

Embodiment 11. A system according to any one of the embodiments 1 to 10, wherein at least some of the microstructures at least one of: attracts at least one substance to the microstructures; repels at least one substance from the microstructures; attracts at least one analyte to the microstructures; and, repels at least one analyte from the microstructures.

Embodiment 12. A system according to embodiment 10 or embodiment 11, wherein an appearance of different parts of the substrate are indicative of at least one of: successful breaching or penetration of the functional barrier; a presence or absence of different analytes; and, different concentrations of analytes.

Embodiment 13. A system according to any one of the embodiments 10 to 12, wherein the appearance of the substrate includes a pattern used to indicate a presence, absence, level or concentration of analytes, and wherein the pattern includes at least one of: an alphanumeric pattern; or, an iconic pattern.

Embodiment 14. A system according to any one of the embodiments 1 to 13, wherein the substrate and microstructures are at least partially transparent to electromagnetic radiation.

Embodiment 15. A system according to any one of the embodiments 1 to 14, wherein at least one of the substrate and the microstructures include at least one of: fabric; woven fabric; electronic fabric; natural fibres; silk; organic materials; natural composite materials; artificial composite materials; ceramics; stainless steel; metal; polymer; silicon; semiconductor; organosilicates; gold; silver; carbon; carbon nano materials; platinum; and, titanium.

Embodiment 16. A system according to any one of the embodiments 1 to 15, wherein the substrate and microstructures include at least one of: the same material; and, different materials.

Embodiment 17. A system according to any one of the embodiments 1 to 16, wherein the substrate is at least one of: at least partially flexible; configured to conform to an outer surface of the functional barrier; and, configured to conform to a shape of at least part of a subject.

Embodiment 18. A system according to any one of the embodiments 1 to 17, wherein at least some of the microstructures are at least one of: blades; ridges; needles; and, plates.

Embodiment 19. A system according to any one of the embodiments 1 to 18, wherein at least some of the microstructures at least one of: are at least partially tapered; have a cross sectional shape that is at least one of: circular; rectangular; cruciform; square rounded square; rounded rectangular; ellipsoidal; and, at least partially hollow; have a surface that is at least partially at least one of: smooth; serrated; includes one or more pores; includes one or more raised portions; and, rough; are at least partially hollow; are porous; and, include an internal structure.

Embodiment 20. A system according to any one of the embodiments 1 to 19, wherein the microstructures include anchor microstructures used to anchor the substrate to the subject.

Embodiment 21. A system according to embodiment 20, wherein the anchor microstructures at least one of: undergo a shape change; undergo a shape change in response to at least one of substances in the subject and applied stimulation; swell; swell in response to at least one of substances in the subject and applied stimulation; include anchoring structures; have a length greater than that of other microstructures; and, enter the dermis.

Embodiment 22. A system according to any one of the embodiments 1 to 21, wherein the microstructures have a length that is at least one of: greater than the thickness of the functional barrier; at least 10% greater than the thickness of the functional barrier; at least 20% greater than the thickness of the functional barrier; at least 50% greater than the thickness of the functional barrier; at least 75% greater than the thickness of the functional barrier; at least 100% greater than the thickness of the functional barrier; no more than 2000% greater than the thickness of the functional barrier; no more than 1000% greater than the thickness of the functional barrier; no more than 500% greater than the thickness of the functional barrier; no more than 100% greater than the thickness of the functional barrier; no more than 75% greater than the thickness of the functional barrier; and, no more than 50% greater than the thickness of the functional barrier.

Embodiment 23. A system according to any one of the embodiments 1 to 22, wherein the microstructures are applied to skin of the subject, and wherein at least some of the microstructures at least one of: penetrate the stratum corneum; enter the viable epidermis but not the dermis; and, enter the dermis.

Embodiment 24. A system according to any one of the embodiments 1 to 23, wherein at least some of the microstructures have a length that is at least one of: less than 2500 μm; less than 1000 μm; less than 750 μm; less than 600 μm; less than 500 μm; less than 400 μm; less than 300 μm; less than 250 μm; greater than 100 μm; greater than 50 μm; and, greater than 10 μm.

Embodiment 25. A system according to any one of the embodiments 1 to 24, wherein at least some of the microstructures have a maximum width that is at least one of: less than 50000 μm; less than 40000 μm; less than 30000 μm; less than 20000 μm; less than 10000 μm; less than 1000 μm; less than 500 μm; less than 100 μm; less than 50 μm; less than 40 μm; less than 30 μm; less than 20 μm; and, less than 10 μm.

Embodiment 26. A system according to any one of the embodiments 1 to 25, wherein at least some of the microstructures have a maximum thickness that is at least one of: less than 1000 μm; less than 500 μm; less than 200 μm; less than 100 μm; less than 50 μm; less than 20 μm; less than 10 μm; at least 1 μm; at least 0.5 μm; and, at least 0.1 μm.

Embodiment 27. A system according to any one of the embodiments 1 to 26, wherein the microstructures have a density that is at least one of: less than 50,000 per cm²; less than 30,000 per cm²; less than 10,000 per cm²; less than 1,000 per cm²; less than 500 per cm²; less than 100 per cm²; less than 10 per cm²; and, less than 5 per cm².

Embodiment 28. A system according to any one of the embodiments 1 to 27, wherein the microstructures have a spacing that is at least one of: less than 20 mm; less than 10 mm; less than 1 mm; less than 0.1 mm; and, less than 10 μm.

Embodiment 29. A system according to any one of the embodiments 1 to 28, wherein microstructures include an at least partially electromagnetically transparent material.

Embodiment 30. A system according to embodiment 29, wherein microstructures include an electromagnetically opaque layer including ports to allow electromagnetic radiation to be emitted from or received by the ports.

Embodiment 31. A system according to embodiment 30, wherein microstructures include electromagnetically reflective layers to allow electromagnetic radiation to be conducted to and from designated ports.

Embodiment 32. A system according to any one of the embodiments 1 to 31, wherein the microstructures are configured to deliver stimulation, and wherein the stimulation is at least one of: biochemical; chemical; mechanical; magnetic; thermal; electrical; electromagnetic; and, optical.

Embodiment 33. A system according to any one of the embodiments 1 to 32, wherein the one or more microstructure electrodes interact with one or more analytes of interest such that a response signal is dependent on a presence, absence, level or concentration of analytes of interest.

Embodiment 34. A system according to any one of the embodiments 1 to 33, wherein the system includes an actuator configured to apply a force to the substrate to at least one of: sense tissue mechanical properties; cause the microstructures to breach the functional barrier; cause the microstructures to penetrate the functional barrier; provide mechanical stimulation; attract substances; repel substances; trigger a biological response; release material from a coating on at least some microstructures; disrupt a coating on at least some microstructures; dissolve a coating on at least some microstructures; and, release a coating on at least some microstructures.

Embodiment 35. A system according to embodiment 34, wherein the actuator is at least one of: an electric actuator; a magnetic actuator; a polymeric actuator; a fabric or woven actuator; a pneumatic actuator; a thermal actuator; a hydraulic actuator; a chemical actuator; a piezoelectric actuator; and, an electromagnetic actuator; and, a mechanical actuator.

Embodiment 36. A system according to embodiment 34 or embodiment 35, wherein the actuator is configured to apply at least one of: a vibratory force; a periodic force; a repeated force; a single continuous force; and, a single instantaneous force.

Embodiment 37. A system according to embodiment 35, wherein the force is applied at a frequency that is at least one of: at least 0.01 Hz; at least 0.1 Hz; at least 1 Hz; at least 10 Hz; at least 50 Hz; at least 100 Hz; at least 1 kHz; at least 10 kHz; at least 100 kHz; varying; varying depending on at least one of: a time of application; a depth of penetration; a degree of penetration; and, an insertion resistance; increasing with an increasing depth of penetration; decreasing with an increasing depth of penetration; increasing until a point of penetration; and decreasing after a point of penetration.

Embodiment 38. A system according to any one of the embodiments 34 to 37, wherein the force is at least one of: at least 0.1 μN; at least 1 μN; at least 5 μN; at least 10 μN; at least 20 μN; at least 50 μN; at least 100 μN; at least 500 μN; at least 1000 μN; at least 10 mN; at least 100 mN; varying depending on at least one of: a time of application; a depth of penetration; a degree of penetration; and, an insertion resistance; increasing with an increasing depth of penetration; decreasing with an increasing depth of penetration; increasing until a point of penetration; and decreasing after a point of penetration.

Embodiment 39. A system according to any one of the embodiments 34 to 38, wherein the actuator is configured to cause movement of the microstructures that is at least one of: greater than 0.001 times a length of the microstructure; greater than 0.01 times a length of the microstructure; greater than 0.1 times a length of the microstructure; greater than a length of the microstructure; greater than 10 times a length of the microstructure; greater than 100 times a length of the microstructure; greater than 1000 times a length of the microstructure; varying depending on at least one of: a time of application; a depth of penetration; a degree of penetration; and, an insertion resistance; increasing with an increasing depth of penetration; decreasing with an increasing depth of penetration; increasing until a point of penetration; and decreasing after a point of penetration.

Embodiment 40. A system according to any one of the embodiments 34 to 39, wherein the system: detects, using response of the actuator, at least one of: a depth of penetration; a degree of penetration; and, an insertion resistance; controls the actuator in accordance with the detection.

Embodiment 41. A system according to any one of the embodiments 34 to 40, wherein the actuator is operatively coupled to the substrate.

Embodiment 42. A system according to any one of the embodiments 1 to 41, wherein the substrate is at least one of: secured to the subject; secured to the subject using anchor microstructures; secured to the subject using an adhesive patch; and, secured to the subject using a strap.

Embodiment 43. A system according to any one of the embodiments 1 to 42, wherein the system is configured to be worn over a time period.

Embodiment 44. A system according to embodiment 43, wherein the time period is at least one of: less than 0.01 seconds; less than 0.1 seconds; less than 1 second; less than 10 seconds; at least one hour; at least one day; and, at least one week.

Embodiment 45. A system according to embodiment 43 or embodiment 44, wherein the microstructures are configured to remain in the subject during the time period.

Embodiment 46. A system according to any one of the embodiments 1 to 45, wherein change in appearance is viewable upon exposure to electromagnetic radiation having a defined wavelength range.

Embodiment 47. A system according to embodiment 46, wherein the defined wavelength range includes at least one of: infrared radiation; ultraviolet radiation; and, visible radiation.

Embodiment 48. A system according to any one of the embodiments 1 to 47, wherein the change in appearance is detectable using an imaging device.

Embodiment 49. A system according to embodiment 48, wherein the system includes one or more electronic processing devices that: receive an image of the substrate from an imaging device; and, analyse the image to determine an indicator indicative of a presence, absence, level or concentration of analytes.

Embodiment 50. A system according to embodiment 49, wherein the one or more electronic processing devices: control a radiation source to selectively expose the substrate to electromagnetic radiation; and, control the imaging device to capture an image in response to exposure of the substrate to electromagnetic radiation.

Embodiment 51. A system according to embodiment 50, wherein the image is captured at least one of: concurrently with exposure of the substrate to electromagnetic radiation; and, a defined time after exposure of the substrate to electromagnetic radiation.

Embodiment 52. A system according to any one of the embodiments 1 to 51, wherein the system is at least partially wearable.

Embodiment 53. A method for detecting analytes in a biological subject, the method including: using at least one substrate including one or more microstructures to breach a functional barrier of the subject; and, using a response of the one or more microstructures to a presence, absence, level or concentration of analytes to determine a change in appearance thereby indicating that a presence, absence, level or concentration of analytes has been detected.

Persons skilled in the art will appreciate that numerous variations and modifications will become apparent. All such variations and modifications which become apparent to persons skilled in the art, should be considered to fall within the spirit and scope that the invention broadly appearing before described.

1) A system for detecting analytes in a biological subject, the system including at least one substrate including a plurality of microstructures configured to breach a stratum corneum of the subject, and wherein the one or more microstructures are responsive to a presence, absence, level or concentration of analytes to cause a change in appearance thereby indicating that a presence, absence, level or concentration of analytes has been detected.

2) A system according to claim 1, wherein the change in appearance includes at least one of:
a) a change in visual appearance;
b) a change in pattern;
c) a change in electromagnetic properties;
d) a colour change;
e) a change in hue;
f) a change in saturation;
g) a change in lightness;
h) a change in chroma;
i) a change in colorfulness;
j) a change in colour constancy;
k) a change in radiance;
l) a change in luminance;
m) a change in brightness;
n) a change in fluorescence;
o) a change in photoluminescence;
p) a change in phosphorescence;
q) a change in reflectivity;
r) a change in refractivity;
s) a reversible change in appearance; and,
t) an irreversible change in appearance.

3) A system according to claim 1, wherein the change in appearance is at least one of:
a) a change in the tissue;
b) a change in the substrate;
c) a change on the one or more microstructures;
d) a change in the one or more microstructures; and,
e) is viewable through the substrate.

4) A system according to claim 1, wherein at least some of the microstructures include at least one of:
a) surface features that cause a change in appearance of the microstructures;
b) a material that undergoes a change in appearance; and,
c) a coating that causes a change in appearance.

5) A system according to claim 1, wherein at least some of the microstructures include a coating and wherein the coating at least one of:
a) undergoes a change in appearance in response to a presence, absence, level or concentration of analytes;
b) causes a change in appearance of surrounding tissue in response to a presence, absence, level or concentration of analytes;
c) causes a change in appearance of analytes; and,
d) dissolves at least one of:
i) after a defined time period;
ii) in response to a presence, absence, level or concentration of analytes; and,
iii) upon breaching or penetration of the functional barrier.

6) A system according to claim 4, wherein at least one of:
a) at least some microstructures are uncoated;
b) at least some microstructures are porous with an internal coating;
c) at least some microstructures are partially coated;
d) different microstructures have different coatings;
e) different parts of microstructures include different coatings; and,
f) at least some microstructures include multiple coatings.

7) A system according to claim 4, wherein the coating at least one of:
a) interacts with analytes;
b) undergoes a shape change to selectively anchor microstructures;
c) modifies surface properties to at least one of:
i) increase hydrophilicity;
ii) increase hydrophobicity; and,
iii) minimize biofouling;
d) attracts at least one substance to the microstructures;
e) repels at least one substance from the microstructures;
f) provides a physical structure to at least one of:
i) facilitate penetration of the barrier;
ii) strengthen the microstructures; and,
iii) anchor the microstructures in the subject;
g) dissolves to at least one of:
i) expose a microstructure;
ii) expose a further coating; and,
iii) expose a material;
h) provides stimulation to the subject;
i) contains a material;
j) selectively releases a material;
k) acts as a barrier to preclude at least one substance from the microstructures; and,
l) includes at least one of:
i) polyethylene;
ii) polyethylene glycol;
iii) polyethylene oxide;
iv) zwitterions;
v) peptides;
vi) hydrogels; and,
vii) self-assembled monolayer.

8) A system according to claim 1, wherein at least some of the microstructures include a material including at least one of:
a) a bioactive material;
b) a reagent for reacting with analytes in the subject;
c) a binding agent for binding with analytes of interest;
d) a material for binding one or more analytes of interest;
e) a probe for selectively targeting analytes of interest;
f) an insulator;
g) a material to reduce biofouling;
h) a material to attract at least one substance to the microstructures;
i) a material to repel at least one substance from the microstructures;
j) a material to attract at least some analytes to the microstructures; and,
k) a material to repel at least some analytes from the microstructures.

9) A system according to claim 1, wherein the substrate includes a plurality of microstructures and wherein different microstructures are at least one of:
a) differentially responsive to analytes;
b) responsive to different analytes;
c) responsive to different combination of analytes; and,
d) responsive to different concentrations of analytes.

10) A system according to claim 1, wherein at least some of the microstructures at least one of:
a) attracts at least one substance to the microstructures;
b) repels at least one substance from the microstructures;
c) attracts at least one analyte to the microstructures; and,
d) repels at least one analyte from the microstructures.

11) A system according to claim 9, wherein an appearance of different parts of the substrate or different microstructures are indicative of at least one of:
a) successful breaching or penetration of the stratum corneum;
b) successful breaching or penetration of a dermal barrier;
c) a presence or absence of different analytes; and,
d) different concentrations of analytes.

12) A system according to claim 9, wherein the appearance of the substrate includes a pattern used to indicate a presence, absence, level or concentration of analytes, and wherein the pattern includes at least one of:
a) an alphanumeric pattern; or,
b) an iconic pattern.

13) A system according to claim 1, wherein the substrate and microstructures are at least partially transparent to electromagnetic radiation.

14) A system according to claim 1, wherein at least one of the substrate and the microstructures include a polymer.

15) A system according to claim 1, wherein the substrate is at least one of:
a) at least partially flexible;
b) configured to conform to an outer surface of the functional barrier; and,
c) configured to conform to a shape of at least part of a subject.

16) A system according to claim 1, wherein at least some of the microstructures are plate microstructures that are at least partially tapered and have a substantially rounded rectangular cross sectional shape.

17) A system according to claim 1, wherein the microstructures include anchor microstructures used to anchor the substrate to the subject and wherein the anchor microstructures at least one of:
a) undergo a shape change;
b) undergo a shape change in response to at least one of substances in the subject and applied stimulation;
c) swell;
d) swell in response to at least one of substances in the subject and applied stimulation;
e) include anchoring structures;
f) have a length greater than that of other microstructures;
g) are rougher than other microstructures;
h) have a higher surface friction than other microstructures;
i) are blunter than other microstructures;
j) are fatter than other microstructures; and,
k) enter the dermis.

18) A system according to claim 1, wherein at least some of the microstructures have at least one of:
a) a length that is at least one of:
i) less than 2500 μm;
ii) less than 1000 μm;
iii) less than 750 μm;
iv) less than 450 μm;
v) less than 300 μm;
vi) less than 250 μm;
vii) about 250 μm;
viii) about 150 μm;
ix) greater than 100 μm;
x) greater than 50 μm; and,
xi) greater than 10 μm;
b) a maximum width that is at least one of:
i) less than 2500 μm;
ii) less than 1000 μm;
iii) less than 750 μm;
iv) less than 450 μm;
v) less than 300 μm;
vi) less than 250 μm;
vii) of a similar order of magnitude to the length;
viii) greater than the length;
ix) greater than the length;
x) about the same as the length;
xi) about 250 μm;
xii) about 150 μm; and,
xiii) greater than 50 μm; and,
c) a maximum thickness that is at least one of:
i) less that the width;
ii) significantly less that the width;
iii) of a smaller order of magnitude to the length;
iv) less than 300 μm;
v) less than 200 μm;
vi) less than 50 μm;
vii) about 25 μm; and,
viii) greater than 10 μm.

19) A system according to claim 1, wherein at least some of the microstructures include at least one of:
a) a shoulder that is configured to abut against the stratum corneum to control a depth of penetration; and,
b) a shaft extending from a shoulder to the tip, the shaft being configured to control a position of the tip in the subject.

20) A system according to claim 1, wherein the microstructures have at least one of:
a) a density that is at least one of:
i) less than 5000 per cm²;
ii) greater than 100 per cm²; and,
iii) about 600 per cm²; and,
b) a spacing that is at least one of:
i) less than 1 mm;
ii) about 0.5 mm;
iii) about 0.2 mm;
iv) about 0.1 mm; and,
v) more than 10 μm.

21) A system according to claim 1, wherein microstructures include an at least partially electromagnetically transparent material.

22) A system according to claim 21, wherein microstructures include an electromagnetically opaque layer including ports to allow electromagnetic radiation to be emitted from or received by the ports.

23) A system according to claim 22, wherein microstructures include electromagnetically reflective layers to allow electromagnetic radiation to be conducted to and from designated ports.

24) A system according to claim 1, wherein the microstructures are configured to deliver stimulation, and wherein the stimulation is at least one of:
a) biochemical;
b) chemical;
c) mechanical;
d) magnetic;
e) thermal;
f) electrical;
g) electromagnetic; and,
h) optical.

25) A system according to claim 1, wherein the system includes an actuator configured to apply a force to the substrate to at least one of pierce and penetrate the stratum corneum.

26) A system according to claim 25, wherein the actuator is at least one of:
a) an electromagnetic actuator;
b) a vibratory motor;
c) a piezoelectric actuator; and,
d) a mechanical actuator.

27) A system according to claim 25, wherein the actuator is configured to apply at least one of:
a) a biasing force;
b) a vibratory force; and,
c) a single continuous force.

28) A system according to claim 25, wherein the force at least one of:
a) includes a continuous force that is at least one of:
i) greater than 1 N;
ii) less than 10 N; and,
iii) about 2.5 to 5 N;
b) includes a vibratory force that is at least one of:
i) at least 1 mN;
ii) about 200 mN; and,
iii) less than 1000 mN; and,
c) is applied at a frequency that is at least one of:
i) at least 10 Hz;
ii) about 100 to 200 Hz; and,
iii) less than 1 kHz.

29) A system according to claim 1, wherein the substrate is at least one of:
a) secured to the subject;
b) secured to the subject using anchor microstructures;
c) secured to the subject using an adhesive patch; and,
d) secured to the subject using a strap.

30) A system according to claim 1, wherein the system is configured to perform measurements over a time period and wherein the microstructures are configured to remain in the subject during the time period.

31) A system according to claim 30, wherein the time period is at least one of:
a) at least one minute;
b) at least one hour;
c) at least one day; and,
d) at least one week.

32) A system according to claim 1, wherein change in appearance is viewable upon exposure to electromagnetic radiation having a defined wavelength range, the defined wavelength range including at least one of:
a) infrared radiation;
b) ultraviolet radiation; and,
c) visible radiation.

33) A system according to claim 1, wherein the change in appearance is detectable using an imaging device.

34) A system according to claim 33, wherein the system includes one or more electronic processing devices that:
a) receive an image of the substrate from an imaging device; and,
b) analyse the image to determine an indicator indicative of a presence, absence, level or concentration of analytes.

35) A system according to claim 34, wherein the one or more electronic processing devices:
a) control a radiation source to selectively expose the substrate to electromagnetic radiation; and,
b) control the imaging device to capture an image in response to exposure of the substrate to electromagnetic radiation.

36) A system according to claim 35, wherein the image is captured at least one of:
a) concurrently with exposure of the substrate to electromagnetic radiation; and,
b) a defined time after exposure of the substrate to electromagnetic radiation.

37) A system according to claim 1, wherein the system is at least partially wearable.

38) A method for detecting analytes in a biological subject, the method including:
a) using at least one substrate including a plurality of microstructures configured to breach a stratum corneum of the subject; and,
using a response of the one or more microstructures to a presence, absence, level or concentration of analytes to determine a change in appearance thereby indicating that a presence, absence, level or concentration of analytes has been detected.
